SEQUENCE LISTING (185 sequences)

SEQ ID NO: 1 - DNA, 20 bp, synthetic construct
  source 1..20: mol_type = other DNA
  misc_difference 2: note = May be uracil
  misc_difference 6: note = May be uracil
  misc_difference 7: note = May be uracil
  misc_difference 8: note = May be uracil
  misc_difference 10: note = May be uracil
  misc_difference 12: note = May be uracil
  misc_difference 17: note = May be uracil
  misc_difference 18: note = May be uracil
atcagtttctgtaggcttcc

SEQ ID NO: 2 - DNA, 20 bp, synthetic construct
  source 1..20: mol_type = other DNA
  misc_difference 5: note = May be uracil
  misc_difference 8: note = May be uracil
  misc_difference 11: note = May be uracil
  misc_difference 12: note = May be uracil
  misc_difference 16: note = May be uracil
  misc_difference 17: note = May be uracil
  misc_difference 20: note = May be uracil
ggcatactggttcagttgat

SEQ ID NO: 3 - DNA, 20 bp, synthetic construct
  source 1..20: mol_type = other DNA
  misc_difference 3: note = May be uracil
  misc_difference 8: note = May be uracil
  misc_difference 16: note = May be uracil
  misc_difference 19: note = May be uracil
  misc_difference 20: note = May be uracil
gctcaggtcaggcagtggtt

SEQ ID NO: 4 - DNA, 20 bp, synthetic construct
  source 1..20: mol_type = other DNA
  misc_difference 8: note = May be uracil
  misc_difference 9: note = May be uracil
  misc_difference 10: note = May be uracil
  misc_difference 11: note = May be uracil
  misc_difference 12: note = May be uracil
  misc_difference 20: note = May be uracil
gagagcctttttgcagagct

SEQ ID NO: 5 - DNA, 20 bp, synthetic construct
  source 1..20: mol_type = other DNA
  misc_difference 1: note = May be uracil
  misc_difference 7: note = May be uracil
  misc_difference 9: note = May be uracil
  misc_difference 11: note = May be uracil
  misc_difference 15: note = May be uracil
  misc_difference 17: note = May be uracil
  misc_difference 20: note = May be uracil
tccagctctgtgcctctagt

SEQ ID NO: 6 - DNA, 20 bp, synthetic construct
  source 1..20: mol_type = other DNA
  misc_difference 2: note = May be uracil
  misc_difference 3: note = May be uracil
  misc_difference 9: note = May be uracil
  misc_difference 10: note = May be uracil
  misc_difference 17: note = May be uracil
  misc_difference 18: note = May be uracil
  misc_difference 20: note = May be uracil
gttccagcttgggcagttgt

SEQ ID NO: 7 - DNA, 20 bp, synthetic construct
  source 1..20: mol_type = other DNA
  misc_difference 7: note = May be uracil
  misc_difference 8: note = May be uracil
  misc_difference 11: note = May be uracil
  misc_difference 14: note = May be uracil
  misc_difference 15: note = May be uracil
  misc_difference 19: note = May be uracil
ggaagcttagtccttggctg

SEQ ID NO: 8 - DNA, 20 bp, synthetic construct
  source 1..20: mol_type = other DNA
atcagtttctgtaggcttcc

SEQ ID NO: 9 - DNA, 20 bp, synthetic construct
  source 1..20: mol_type = other DNA
ggcatactggttcagttgat

SEQ ID NO: 10 - DNA, 20 bp, synthetic construct
  source 1..20: mol_type = other DNA
gctcaggtcaggcagtggtt

SEQ ID NO: 11 - DNA, 20 bp, synthetic construct
  source 1..20: mol_type = other DNA
gagagcctttttgcagagct

SEQ ID NO: 12 - DNA, 20 bp, synthetic construct
  source 1..20: mol_type = other DNA
tccagctctgtgcctctagt

SEQ ID NO: 13 - DNA, 20 bp, synthetic construct
  source 1..20: mol_type = other DNA
gttccagcttgggcagttgt

SEQ ID NO: 14 - DNA, 20 bp, synthetic construct
  source 1..20: mol_type = other DNA
ggaagcttagtccttggctg

SEQ ID NO: 15 - RNA, 20 nt, synthetic construct
  source 1..20: mol_type = other RNA
  misc_difference 8: note = May be thymine
  misc_difference 17: note = May be thymine
  misc_difference 20: note = May be thymine
ggaagcctacagaaactgat

SEQ ID NO: 16 - RNA, 20 nt, synthetic construct
  source 1..20: mol_type = other RNA
  misc_difference 2: note = May be thymine
  misc_difference 7: note = May be thymine
  misc_difference 15: note = May be thymine
  misc_difference 17: note = May be thymine
atcaactgaaccagtatgcc

SEQ ID NO: 17 - RNA, 20 nt, synthetic construct
  source 1..20: mol_type = other RNA
  misc_difference 7: note = May be thymine
  misc_difference 11: note = May be thymine
  misc_difference 16: note = May be thymine
aaccactgcctgacctgagc

SEQ ID NO: 18 - RNA, 20 nt, synthetic construct
  source 1..20: mol_type = other RNA
  misc_difference 4: note = May be thymine
  misc_difference 6: note = May be thymine
  misc_difference 17: note = May be thymine
  misc_difference 19: note = May be thymine
agctctgcaaaaaggctctc

SEQ ID NO: 19 - RNA, 20 nt, synthetic construct
  source 1..20: mol_type = other RNA
  misc_difference 3: note = May be thymine
  misc_difference 17: note = May be thymine
actagaggcacagagctgga

SEQ ID NO: 20 - RNA, 20 nt, synthetic construct
  source 1..20: mol_type = other RNA
  misc_difference 6: note = May be thymine
  misc_difference 15: note = May be thymine
acaactgcccaagctggaac

SEQ ID NO: 21 - RNA, 20 nt, synthetic construct
  source 1..20: mol_type = other RNA
  misc_difference 12: note = May be thymine
  misc_difference 17: note = May be thymine
  misc_difference 18: note = May be thymine
cagccaaggactaagcttcc

SEQ ID NO: 22 - RNA, 20 nt, synthetic construct
  source 1..20: mol_type = other RNA
ggaagcctacagaaactgat

SEQ ID NO: 23 - RNA, 20 nt, synthetic construct
  source 1..20: mol_type = other RNA
atcaactgaaccagtatgcc

SEQ ID NO: 24 - RNA, 20 nt, synthetic construct
  source 1..20: mol_type = other RNA
aaccactgcctgacctgagc

SEQ ID NO: 25 - RNA, 20 nt, synthetic construct
  source 1..20: mol_type = other RNA
agctctgcaaaaaggctctc

SEQ ID NO: 26 - RNA, 20 nt, synthetic construct
  source 1..20: mol_type = other RNA
actagaggcacagagctgga

SEQ ID NO: 27 - RNA, 20 nt, synthetic construct
  source 1..20: mol_type = other RNA
acaactgcccaagctggaac

SEQ ID NO: 28 - RNA, 20 nt, synthetic construct
  source 1..20: mol_type = other RNA
cagccaaggactaagcttcc

SEQ ID NO: 29 - RNA, 20 nt, synthetic construct
  source 1..20: mol_type = other RNA
tccctcaccttgaatgaaga

SEQ ID NO: 30 - DNA, 20 bp, synthetic construct
  source 1..20: mol_type = other DNA
gggtggctcctccaatgata

SEQ ID NO: 31 - DNA, 20 bp, synthetic construct
  source 1..20: mol_type = other DNA
gggctccttggcagatgtct

SEQ ID NO: 32 - DNA, 20 bp, synthetic construct
  source 1..20: mol_type = other DNA
tgccagcagccagcagtcac

SEQ ID NO: 33 - DNA, 20 bp, synthetic construct
  source 1..20: mol_type = other DNA
gtgagggaggcaatggctgc

SEQ ID NO: 34 - DNA, 20 bp, synthetic construct
  source 1..20: mol_type = other DNA
tcttcattcaaggtgaggga

SEQ ID NO: 35 - DNA, 20 bp, synthetic construct
  source 1..20: mol_type = other DNA
gagccaggatttcttcattc

SEQ ID NO: 36 - DNA, 20 bp, synthetic construct
  source 1..20: mol_type = other DNA
tccctgcatagttttcctgg

SEQ ID NO: 37 - DNA, 20 bp, synthetic construct
  source 1..20: mol_type = other DNA
ccaccacctccacccactcg

SEQ ID NO: 38 - DNA, 20 bp, synthetic construct
  source 1..20: mol_type = other DNA
gaagccctcagtggtggcac

SEQ ID NO: 39 - DNA, 20 bp, synthetic construct
  source 1..20: mol_type = other DNA
gggcttcttcaactcatacc

SEQ ID NO: 40 - DNA, 20 bp, synthetic construct
  source 1..20: mol_type = other DNA
tcttgaacaggttgggaggg

SEQ ID NO: 41 - DNA, 20 bp, synthetic construct
  source 1..20: mol_type = other DNA
gagcagccaaggagagagcc

SEQ ID NO: 42 - DNA, 20 bp, synthetic construct
  source 1..20: mol_type = other DNA
gggtctatagtgttccagct

SEQ ID NO: 43 - DNA, 20 bp, synthetic construct
  source 1..20: mol_type = other DNA
gaattctccatcttcatgcc

SEQ ID NO: 44 - DNA, 20 bp, synthetic construct
  source 1..20: mol_type = other DNA
gtgcctcaggaagtcactga

SEQ ID NO: 45 - DNA, 20 bp, synthetic construct
  source 1..20: mol_type = other DNA
agtttccacttcttgtaggt

SEQ ID NO: 46 - DNA, 20 bp, synthetic construct
  source 1..20: mol_type = other DNA
ctccagttcccatccatttt

SEQ ID NO: 47 - DNA, 20 bp, synthetic construct
  source 1..20: mol_type = other DNA
ggtactgagggttcatccag

SEQ ID NO: 48 - DNA, 20 bp, synthetic construct
  source 1..20: mol_type = other DNA
gtctgcccacttaactcctc

SEQ ID NO: 49 - DNA, 20 bp, synthetic construct
  source 1..20: mol_type = other DNA
ggtggatgttggtctgccca

SEQ ID NO: 50 - DNA, 20 bp, synthetic construct
  source 1..20: mol_type = other DNA
gtttttgctgaggtggatgt

SEQ ID NO: 51 - DNA, 20 bp, synthetic construct
  source 1..20: mol_type = other DNA
actcttcaagattggcctcg

SEQ ID NO: 52 - DNA, 20 bp, synthetic construct
  source 1..20: mol_type = other DNA
ccatcatcaatgtcatcctc

SEQ ID NO: 53 - DNA, 20 bp, synthetic construct
  source 1..20: mol_type = other DNA
gtctcctgaatccatcatca

SEQ ID NO: 54 - DNA, 20 bp, synthetic construct
  source 1..20: mol_type = other DNA
ctgggcaaacagtctcctga

SEQ ID NO: 55 - DNA, 20 bp, synthetic construct
  source 1..20: mol_type = other DNA
tggtctgcagctcaaaggca

SEQ ID NO: 56 - DNA, 20 bp, synthetic construct
  source 1..20: mol_type = other DNA
gctttgctagaacccttctc

SEQ ID NO: 57 - DNA, 20 bp, synthetic construct
  source 1..20: mol_type = other DNA
atgtagaactccttcagccc

SEQ ID NO: 58 - DNA, 20 bp, synthetic construct
  source 1..20: mol_type = other DNA
gggcatcttgaaacctgctt

SEQ ID NO: 59 - DNA, 20 bp, synthetic construct
  source 1..20: mol_type = other DNA
ggtggagttgacagggcatc

SEQ ID NO: 60 - DNA, 20 bp, synthetic construct
  source 1..20: mol_type = other DNA
gagctctattgttccagtat

SEQ ID NO: 61 - DNA, 20 bp, synthetic construct
  source 1..20: mol_type = other DNA
agtcttcaggcagattagtt

SEQ ID NO: 62 - DNA, 20 bp, synthetic construct
  source 1..20: mol_type = other DNA
gctgttaaatacctccctgc

SEQ ID NO: 63 - DNA, 20 bp, synthetic construct
  source 1..20: mol_type = other DNA
tccctttggtctttgtaaga

SEQ ID NO: 64 - DNA, 20 bp, synthetic construct
  source 1..20: mol_type = other DNA
actagtggtggtgaagttca

SEQ ID NO: 65 - DNA, 20 bp, synthetic construct
  source 1..20: mol_type = other DNA
ggagccactcttgccagtgt

SEQ ID NO: 66 - DNA, 20 bp, synthetic construct
  source 1..20: mol_type = other DNA
tcccttcatgcctctgcctt

SEQ ID NO: 67 - DNA, 20 bp, synthetic construct
  source 1..20: mol_type = other DNA
gtccctcatctcttccaccc

SEQ ID NO: 68 - DNA, 20 bp, synthetic construct
  source 1..20: mol_type = other DNA
ggcctctgttcctggtgctt

SEQ ID NO: 69 - DNA, 20 bp, synthetic construct
  source 1..20: mol_type = other DNA
gcagggtcagccagtggagg

SEQ ID NO: 70 - DNA, 20 bp, synthetic construct
  source 1..20: mol_type = other DNA
gtcccaggcctttcctttcc

SEQ ID NO: 71 - DNA, 20 bp, synthetic construct
  source 1..20: mol_type = other DNA
tcctttgctgtgttcctccc

SEQ ID NO: 72 - DNA, 20 bp, synthetic construct
  source 1..20: mol_type = other DNA
ggcccttctcctcctttgct

SEQ ID NO: 73 - DNA, 20 bp, synthetic construct
  source 1..20: mol_type = other DNA
ccagtgtcaggccacccagc

SEQ ID NO: 74 - DNA, 20 bp, synthetic construct
  source 1..20: mol_type = other DNA
tccttccaactctccctcct

SEQ ID NO: 75 - DNA, 20 bp, synthetic construct
  source 1..20: mol_type = other DNA
ccttctgcaccagaccctcc

SEQ ID NO: 76 - DNA, 20 bp, synthetic construct
  source 1..20: mol_type = other DNA
gttggtgcattcctggctcc

SEQ ID NO: 77 - DNA, 20 bp, synthetic construct
  source 1..20: mol_type = other DNA
gcctccacatccctcactcc

SEQ ID NO: 78 - DNA, 20 bp, synthetic construct
  source 1..20: mol_type = other DNA
gcaggttggtgctggctgtc

SEQ ID NO: 79 - DNA, 20 bp, synthetic construct
  source 1..20: mol_type = other DNA
gtgacaggcccagcccatcc

SEQ ID NO: 80 - DNA, 20 bp, synthetic construct
  source 1..20: mol_type = other DNA
cccagccatgcttctgtcct

SEQ ID NO: 81 - DNA, 20 bp, synthetic construct
  source 1..20: mol_type = other DNA
gcctcagtctgtccagcacc

SEQ ID NO: 82 - DNA, 20 bp, synthetic construct
  source 1..20: mol_type = other DNA
gcctgacacctgcctcagtc

SEQ ID NO: 83 - DNA, 20 bp, synthetic construct
  source 1..20: mol_type = other DNA
ggtgccagtgctaggttctc

SEQ ID NO: 84 - DNA, 20 bp, synthetic construct
  source 1..20: mol_type = other DNA
gcctggtccctctctgagcc

SEQ ID NO: 85 - DNA, 20 bp, synthetic construct
  source 1..20: mol_type = other DNA
agcagccagcagtcacctgt

SEQ ID NO: 86 - DNA, 20 bp, synthetic construct
  source 1..20: mol_type = other DNA
ggagcagtgggttcccttgg

SEQ ID NO: 87 - DNA, 20 bp, synthetic construct
  source 1..20: mol_type = other DNA
ggctggaggcaccatcaccc

SEQ ID NO: 88 - DNA, 20 bp, synthetic construct
  source 1..20: mol_type = other DNA
gctctgtggccctggtatgc

SEQ ID NO: 89 - DNA, 20 bp, synthetic construct
  source 1..20: mol_type = other DNA
ccttgccctgctcctgatgt

SEQ ID NO: 90 - DNA, 20 bp, synthetic construct
  source 1..20: mol_type = other DNA
gtcccttgtcctctcaggcc

SEQ ID NO: 91 - DNA, 20 bp, synthetic construct
  source 1..20: mol_type = other DNA
gcagttctctctctggctcc

SEQ ID NO: 92 - DNA, 20 bp, synthetic construct
  source 1..20: mol_type = other DNA
aggagcccagccctgagcag

SEQ ID NO: 93 - DNA, 20 bp, synthetic construct
  source 1..20: mol_type = other DNA
gctctccaccatccaggcca

SEQ ID NO: 94 - DNA, 20 bp, synthetic construct
  source 1..20: mol_type = other DNA
gttccctgcctctacctccc

SEQ ID NO: 95 - DNA, 20 bp, synthetic construct
  source 1..20: mol_type = other DNA
gctcctccctgctggtgatc

SEQ ID NO: 96 - DNA, 20 bp, synthetic construct
  source 1..20: mol_type = other DNA
gctggccctggactcttctt

SEQ ID NO: 97 - DNA, 20 bp, synthetic construct
  source 1..20: mol_type = other DNA
cccaccctccatctgtgcag

SEQ ID NO: 98 - DNA, 20 bp, synthetic construct
  source 1..20: mol_type = other DNA
tcctgcttttcccactggct

SEQ ID NO: 99 - DNA, 20 bp, synthetic construct
  source 1..20: mol_type = other DNA
agcccttgagtgtgggtcca

SEQ ID NO: 100 - DNA, 20 bp, synthetic construct
  source 1..20: mol_type = other DNA
ctgtccaccttcaccctgcc

SEQ ID NO: 101 - DNA, 20 bp, synthetic construct
  source 1..20: mol_type = other DNA
gccctctgtggccttccctg

SEQ ID NO: 102 - DNA, 20 bp, synthetic construct
  source 1..20: mol_type = other DNA
gccttagacctgccctcctc

SEQ ID NO: 103 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gggtggctcctccaatgata

SEQ ID NO: 104 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gggctccttggcagatgtct

SEQ ID NO: 105 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
tgccagcagccagcagtcac

SEQ ID NO: 106 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gtgagggaggcaatggctgc

SEQ ID NO: 107 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tcttcattcaaggtgaggga

SEQ ID NO: 108 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gagccaggatttcttcattc

SEQ ID NO: 109 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tccctgcatagttttcctgg

SEQ ID NO: 110 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ccaccacctccacccactcg

SEQ ID NO: 111 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gaagccctcagtggtggcac

SEQ ID NO: 112 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gggcttcttcaactcatacc

SEQ ID NO: 113 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tcttgaacaggttgggaggg

SEQ ID NO: 114 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gagagcctttttgcagagct

SEQ ID NO: 115 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gagcagccaaggagagagcc

SEQ ID NO: 116 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
atcagtttctgtaggcttcc

SEQ ID NO: 117 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gttccagcttgggcagttgt

SEQ ID NO: 118 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gggtctatagtgttccagct

SEQ ID NO: 119 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gaattctccatcttcatgcc

SEQ ID NO: 120 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gtgcctcaggaagtcactga

SEQ ID NO: 121 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
agtttccacttcttgtaggt

SEQ ID NO: 122 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ctccagttcccatccatttt

SEQ ID NO: 123 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ggtactgagggttcatccag

SEQ ID NO: 124 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gtctgcccacttaactcctc

SEQ ID NO: 125 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ggtggatgttggtctgccca

SEQ ID NO: 126 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gtttttgctgaggtggatgt

SEQ ID NO: 127 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
actcttcaagattggcctcg

SEQ ID NO: 128 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
ccatcatcaatgtcatcctc

SEQ ID NO: 129 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gtctcctgaatccatcatca

SEQ ID NO: 130 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ctgggcaaacagtctcctga

SEQ ID NO: 131 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tggtctgcagctcaaaggca

SEQ ID NO: 132 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gctttgctagaacccttctc

SEQ ID NO: 133 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
atgtagaactccttcagccc

SEQ ID NO: 134 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gggcatcttgaaacctgctt

SEQ ID NO: 135 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
ggtggagttgacagggcatc

SEQ ID NO: 136 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gagctctattgttccagtat

SEQ ID NO: 137 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
agtcttcaggcagattagtt

SEQ ID NO: 138 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ggaagcttagtccttggctg

SEQ ID NO: 139 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gctgttaaatacctccctgc

SEQ ID NO: 140 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tccctttggtctttgtaaga

SEQ ID NO: 141 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ggcatactggttcagttgat

SEQ ID NO: 142 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
actagtggtggtgaagttca

SEQ ID NO: 143 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ggagccactcttgccagtgt

SEQ ID NO: 144 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tcccttcatgcctctgcctt

SEQ ID NO: 145 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gtccctcatctcttccaccc

SEQ ID NO: 146 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ggcctctgttcctggtgctt

SEQ ID NO: 147 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gcagggtcagccagtggagg

SEQ ID NO: 148 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gtcccaggcctttcctttcc

SEQ ID NO: 149 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
tcctttgctgtgttcctccc

SEQ ID NO: 150 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ggcccttctcctcctttgct

SEQ ID NO: 151 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
ccagtgtcaggccacccagc

SEQ ID NO: 152 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tccttccaactctccctcct

SEQ ID NO: 153 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
ccttctgcaccagaccctcc

SEQ ID NO: 154 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gttggtgcattcctggctcc

SEQ ID NO: 155 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gcctccacatccctcactcc

SEQ ID NO: 156 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gcaggttggtgctggctgtc

SEQ ID NO: 157 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gtgacaggcccagcccatcc

SEQ ID NO: 158 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
cccagccatgcttctgtcct

SEQ ID NO: 159 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gcctcagtctgtccagcacc

SEQ ID NO: 160 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gcctgacacctgcctcagtc

SEQ ID NO: 161 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
ggtgccagtgctaggttctc

SEQ ID NO: 162 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gcctggtccctctctgagcc

SEQ ID NO: 163 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
agcagccagcagtcacctgt

SEQ ID NO: 164 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ggagcagtgggttcccttgg

SEQ ID NO: 165 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
ggctggaggcaccatcaccc

SEQ ID NO: 166 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = 5-methyl-2'-deoxycytidine with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gctctgtggccctggtatgc

SEQ ID NO: 167 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
ccttgccctgctcctgatgt

SEQ ID NO: 168 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gtcccttgtcctctcaggcc

SEQ ID NO: 169 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gcagttctctctctggctcc

SEQ ID NO: 170 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
aggagcccagccctgagcag

SEQ ID NO: 171 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gctctccaccatccaggcca

SEQ ID NO: 172 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gttccctgcctctacctccc

SEQ ID NO: 173 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gctcaggtcaggcagtggtt

SEQ ID NO: 174 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = 5-methyl-2'-deoxycytidine with phosphorothioate linkage
  modified_base 8: mod_base = OTHER; note = 5-methyl-2'-deoxycytidine with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gctcctccctgctggtgatc

SEQ ID NO: 175 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gctggccctggactcttctt

SEQ ID NO: 176 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = 5-methyl-2'-deoxycytidine with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
cccaccctccatctgtgcag

SEQ ID NO: 177 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl-2'-deoxycytidine with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tcctgcttttcccactggct

SEQ ID NO: 178 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
agcccttgagtgtgggtcca

SEQ ID NO: 179 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = 5-methyl-2'-deoxycytidine with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tccagctctgtgcctctagt

SEQ ID NO: 180 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 5-methyl-2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = 5-methyl-2'-deoxycytidine with phosphorothioate linkage
  modified_base 9: mod_base = OTHER; note = 5-methyl-2'-deoxycytidine with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
ctgtccaccttcaccctgcc

SEQ ID NO: 181 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 5-methyl-2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = 5-methyl-2'-deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl-2'-deoxycytidine with phosphorothioate linkage
  modified_base 13: mod_base = OTHER; note = 5-methyl-2'-deoxycytidine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gccctctgtggccttccctg

SEQ ID NO: 182 - DNA, 20 bp, synthetic construct
  source 1..20: mol_type = other DNA
  modified_base 1: mod_base = OTHER; note = 5' hydroxyl 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl-2'-deoxycytidine with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl-2'-deoxycytidine with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 5-methyl-2'-O-methoxyethyl nucleotide with phosphorothioate linkage
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..5: note = RNA
  misc_feature 6..15: note = DNA
  misc_feature 16..20: note = RNA
gccttagacctgccctcctc

SEQ ID NO: 183 - DNA, 20 bp, synthetic construct
  source 1..20: mol_type = other DNA
  modified_base 1: mod_base = OTHER; note = 5' hydroxyl 5-methyl -'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl-2'-deoxycytidine with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl-2'-deoxycytidine with phosphorothioate linkage
  modified_base 15: mod_base = OTHER; note = 5-methyl-2'-deoxycytidine with phosphorothioate linkage
  modified_base 16: mod_base = OTHER; note = 2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
cctataggactatccaggaa

SEQ ID NO: 184 - DNA, 20 bp, synthetic construct
  source 1..20: mol_type = other DNA
  modified_base 1: mod_base = OTHER; note = 2'-O-methoxyethyl nucleotide with phosphorothioate linkage
  modified_base 2: mod_base = OTHER; note = 2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
tgcaagtctgacgcccatct

SEQ ID NO: 185 - DNA, 20 bp, synthetic construct
  source 1..20: mol_type = other DNA
  modified_base 1: mod_base = OTHER; note = 2'-O-methoxyethyl nucleotide with phosphorothioate linkage
  modified_base 2: mod_base = OTHER; note = 5-methyl-2'-O-methoxyethyl nucleotide with phosphorothioate linkage
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl nucleotide with phosphorothioate linkage
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl-2'-O-methoxyethyl nucleotide with phosphorothioate linkage
  modified_base 17: mod_base = OTHER; note = 2'-O-methoxyethyl nucleotide with phosphorothioate linkage
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
accagtgcattcattcgagt

(19) United States
(12) Patent Application Publication
(10) Pub. No.: US 2024/0102017 A1
(43) Pub. Date: Mar. 28, 2024

(54) OLIGONUCLEOTIDE COMPOSITIONS AND METHODS THEREOF

(71) Applicant: Amylyx Pharmaceuticals, Inc., Cambridge, MA (US)

(72) Inventors: Joshua Barry Cohen, Somerville, MA (US); Justin Bernard Klee, Cambridge, MA (US); Duncan Brown, Berkeley, CA (US)

(21) Appl. No.: 18/314,761

(22) Filed: May 9, 2023

Related U.S. Application Data
(60) Provisional application No. 63/397,707, filed on Aug. 12, 2022.
(60) Provisional application No. 63/340,365, filed on May 10, 2022.

(51) Int. Cl. C12N 15/113; A61P 25/28

(52) U.S. Cl. CPC C12N 15/1137; A61P 25/28; C12N 2310/14; C12N 2310/315; C12N 2310/321

(57) ABSTRACT

Among other things, the present disclosure provides oligonucleotides targeting calpain-2 and compositions thereof. In some embodiments, the present disclosure provides methods for preventing or treating various conditions, disorders or diseases.

### CROSS-REFERENCE TO RELATED APPLICATIONS

This application claims priority to U.S. Provisional Application Ser. No. 63/340,365, filed May 10, 2022, and 63/397,707, filed Aug. 12, 2022, the entirety of each of which is incorporated herein by reference.

### SEQUENCE LISTING

This application contains a Sequence Listing that has been submitted electronically as an XML file named “Sequence Listing.xmL” The XML file, created on Sep. 13, 2023, is 1,246,833 bytes in size. The material in the XML file is hereby incorporated by reference in its entirety.

### BACKGROUND

Oligonucleotides are useful in various applications, e.g., therapeutic, diagnostic, and/or research applications. For example, oligonucleotides targeting various genes can be useful for treatment of conditions, disorders or diseases related to such target genes.

### SUMMARY

Among other things, the present disclosure provides technologies (e.g., oligonucleotides, compositions, methods, etc.) for treating various conditions, disorders or diseases associated with calpain-2. In some embodiments, the present disclosure provides oligonucleotides that comprise various modifications, e.g., nucleobase modifications, sugar modifications, internucleotidic linkage modifications, etc. and can hybridize to a calpain-2 transcript. In some embodiments, the present disclosure provides oligonucleotides and compositions thereof that when administered or delivered to a system comprising or expressing a calpain-2 transcript can reduce the level of a calpain-2 transcript. In some embodiments, a provided technology reduces levels of a calpain-2 transcript and/or polypeptide in a system. In some embodiments, the present disclosure provides technologies for preventing and/or treating various conditions, disorders or diseases associated with calpain-2.

In some embodiments, the present disclosure encompasses the recognition that oligonucleotides of certain base sequence may be more effective in reducing levels of calpain-2 transcripts (e.g., calpain-2 mRNA) and/or products thereof (e.g., calpain-2 polypeptides). In some embodiments, a base sequence of an oligonucleotide is comprises about 5 or more (e.g., about 5, 6, 7, 8, 9, 10, 11, 12, 13, 14, 15, 16, 17, 18, 19, or 20) contiguous nucleobases of ATCAGTTTCTGTAGGCTTTCC (SEQ ID NO: 1), GGCATACTGGTTCAGTGAT (SEQ ID NO: 2), GCTCAGGTCAGGCAGTGGTT (SEQ ID NO: 3), GAGAGCCTTTTTGCAGAGCT (SEQ ID NO: 4), TCCAGCTCTGTGCCTCTAGT (SEQ ID NO: 5), GTTCCAGCTTGGGCAGTTGT (SEQ ID NO: 6) or GGAAGCTTAGTCCTTGGCTG (SEQ ID NO: 7), wherein each T is optionally and independently replaced with U. In some embodiments, a base sequence of an oligonucleotide is ATCAGTTCTGTAGGCTTCC (SEQ ID NO: 8), GGCATACTGGTTCAGTTGAT (SEQ ID NO: 9), GCTCAGGTCAGGCAGTGGTT (SEQ ID NO: 10), GAGAGCCTTTTTGCAGAGCT (SEQ ID NO: 11), TCCAGCTCTGTGCCTCTAGT (SEQ ID NO: 12), GTTCCAGCTTGGGCAGTTGT (SEQ ID NO: 13) or GGAAGCTTAGTCCTTGGCTG (SEQ ID NO: 14).

In some embodiments, provided oligonucleotides comprising various modifications, such as nucleobase modifications, sugar modifications, internucleotidic linkage modifications, etc. Various useful modifications are available in the art and may be utilized in accordance with the present disclosure. In some embodiments, modifications provide various benefits, e.g., improved stability, binding affinity, pharmacokinetic profiles, pharmacodynamic profiles, etc.

For example, in some embodiments, provided oligonucleotides comprise various sugar modifications. In some embodiments, a modified sugar is a natural RNA sugar with a 2′-OR^{s} modification, wherein R^{s} is an optionally substituted C₁₋₆ aliphatic, and —OR^{s} replace the 2′-OH group (a “2′-OR^{s} modified sugar”). In some embodiments. R^{s} is optionally substituted C₁₋₆ alkyl. In some embodiments, R^{s} is —CH₃. In some embodiments, R^{s} is —CH₂CH₂OCH₃.

In some embodiments, provided oligonucleotides comprises or consists of a wing-core-wing structure, wherein there are independently about 1-10 (e.g., about 1, 2, 3, 4, 5, 6, 7, 8, 9, or 10) nucleosides in each wing, there are about 5 or more (e.g., about 5-20, about 5, 6, 7, 8, 9, 10, 11, 12, 13, 14, 15, 16, 17, 18, 19, or 20, etc.) nucleosides in a core, and each wing independently comprises one or more (e.g., about 1, 2, 3, 4, 5, 6, 7, 8, 9, 10 or more) modified sugars. In some embodiments, each sugar in a wing is independently a modified sugar. In some embodiments, each sugar in a wing is independently a 2′-OR^{s} modified sugar. In some embodiments, a modified sugar is a 2′-MOE modified sugar (a 2′-OR^{s} modified sugar wherein R^{s} is —CH₂CH₂OCH₃). In some embodiments, each wing independently comprises one or more (e.g., about 1, 2, 3, 4, 5 or more) 2′-MOE modified sugar. In some embodiments, each sugar in a wing is independently a 2′-MOE modified sugar. In some embodiments, core regions comprise fewer modified sugars, and/or lower levels of modified sugars, compared to one or both wings. In some embodiments, there are no modified sugars in a core region. In some embodiments, each sugar in a core region is independently a nature DNA sugar.

Additionally or alternatively, in some embodiments, provided oligonucleotides comprise modified internucleotidic linkages. In some embodiments, modified internucleotidic linkages provide improved properties and/or activities compared to natural phosphate linkages. Various internucleotidic linkages are available in the art and can be utilized in accordance with the present disclosure. In some embodiments, a modified internucleotidic linkage is a phosphorothioate internucleotidic linkage (—O—P(O)(SH)—O—, which may exist in various salt forms). In some embodiments, each linkages in a provided oligonucleotide is a phosphorothioate internucleotidic linkage.

In some embodiments, the present disclosure provides technologies for preparing oligonucleotides and compositions thereof. In some embodiments, provided oligonucleotides and compositions thereof are of high purity. In some embodiments, oligonucleotides are provided as diastereomeric mixtures with respect to chiral linkage phosphorus, e.g., in phosphorothioate internucleotidic linkages. In some embodiments, one or more diastereomers with respect to chiral linkage phosphorus are enriched in provided compositions.

As described herein, oligonucleotides and compositions of the present disclosure may be provided/utilized in various forms. In some embodiments, the present disclosure provides compositions comprising one or more forms of oligonucleotides, e.g., acid forms (e.g., in which natural phosphate linkages exist as —O(P(O)(OH)—O—, phosphorothioate internucleotidic linkages exist as —O(P(O)(SH)—O—)), salt forms (e.g., in which one or more or all natural phosphate linkages independently exist as salt forms (e.g., sodium salt (—O(P(O)(O⁻Na⁺)—O—), one or more or all phosphorothioate internucleotidic linkages exist as salt forms (e.g., sodium salt (—O(P(O)(S⁻ Na⁺)—O—), etc.), hydrates, etc. As appreciated by those skilled in the art, oligonucleotides can exist in various salt forms, including pharmaceutically acceptable salts, and in solutions (e.g., various aqueous buffering system), cations may dissociate from anions. In some embodiments, the present disclosure provides a pharmaceutical composition comprising a provided oligonucleotide and/or one or more pharmaceutically acceptable salts thereof, and a pharmaceutically acceptable carrier. In some embodiments, a pharmaceutically acceptable carrier is or comprises a buffer. In some embodiments, a pharmaceutically acceptable carrier is a buffered saline. In some embodiments, a pharmaceutically acceptable carrier is artificial cerebrospinal fluid (aCSF). In some embodiments, a pharmaceutically acceptable carrier is cerebrospinal fluid.

In some embodiments, the present disclosure describes useful technologies for assessing oligonucleotide and compositions thereof. Certain useful technologies are described in the Examples.

Provided technologies can be utilized for various purposes. For example, in some embodiments, provided technologies are useful for preventing and/or treating various conditions, disorders or diseases associated with calpain-2. In some embodiments, the present disclosure provides a method for preventing a condition, disorder or disease, comprising administering or delivering to a subject susceptible thereto an effective amount of a provided oligonucleotide. In some embodiments, the present disclosure provides a method for treating a condition, disorder or disease, comprising administering or delivering to a subject suffering therefrom an effective amount of a provided oligonucleotide. In some embodiments, an oligonucleotide is administered or delivered in a pharmaceutical composition. In some embodiments, an oligonucleotide is administered or delivered in one or more forms, e.g., in some embodiments, in one or more pharmaceutically acceptable salt forms. In some embodiments, an oligonucleotide is administered or delivered in a solution, e.g., in an aCSF solution. Various technologies are available in the art and may be utilized to administer or deliver provided oligonucleotides and compositions thereof. For example, in some embodiments, oligonucleotides and compositions there are administered or delivered intrathecally.

In some embodiments, a condition, disorder or disease is a neurodegenerative condition, disorder or disease. In some embodiments, a condition, disorder or disease is or comprises Wallerian degeneration. In some embodiments, a condition, disorder or disease is associated with Wallerian degeneration. In some embodiments, a condition, disorder or disease is amyotrophic lateral sclerosis (ALS). In some embodiments, a condition, disorder or disease is neuropathy. In some embodiments, a condition, disorder or disease is peripheral neuropathy. In some embodiments, a condition, disorder or disease is peripheral neuropathy induced by chemotherapy. In some embodiments, a condition, disorder or disease is Parkinson's disease. In some embodiments, a condition, disorder or disease is Huntington's disease. In some embodiments, a condition, disorder or disease is Alzheimer's disease. In some embodiments, a condition, disorder or disease is frontotemporal dementia. In some embodiments, a condition, disorder or disease is brain injury. In some embodiments, a condition, disorder or disease is traumatic brain injury. In some embodiments, a condition, disorder or disease is progressive supranuclear palsy. In some embodiments, a condition, disorder or disease is corticobasal degeneration. In some embodiments, a condition, disorder or disease is Wolfram Syndrome. In some embodiments, a condition, disorder or disease is Friedreich's Ataxia. In some embodiments, a condition, disorder or disease is Multiple System Atrophy. In some embodiments, a condition, disorder or disease is Spinal Cerebellar Ataxia. In some embodiments, a condition, disorder or disease is Spinal Muscular Atrophy (SMA). In some embodiments, a condition, disorder or disease is Pick's Disease. In some embodiments, a condition, disorder or disease is progressive motor atrophy.

### BRIEF DESCRIPTION OF THE DRAWINGS

FIG. 1. Provided oligonucleotides can reduce levels of calpain-2 (CAPN2) mRNA. (A) and (B): Various oligonucleotides were demonstrated to knockdown CAPN2. Human iPSC-derived glutamatergic neurons were treated with various oligonucleotides targeting CAPN2 by gymnotic uptake for 48 hours. Cells were lysed and RNA collected. RNA was used in real time RT-qPCR to quantify fold-change of CAPN2 expression. Average fold-change of CAPN2 expression of biological replicates (black dots) is represented in the top panel, average CAPN2 Cp value of technical replicates (black dots) is represented in the middle panel, and average RPLP0 Cp value of technical replicates (black dots) is represented in the bottom panel. The X-axis denotes oligonucleotide; the Y-axis depicts fold-change in CAPN2 expression (top panel), CAPN2 Cp value (middle panel), or RPLP0 Cp value (bottom panel). Dotted horizontal line indicates corresponding fold-change or Cp value from treatment with vehicle-only negative control. Error bars represent standard deviation.

FIG. 2. Various provided oligonucleotides provide knockdown of calpain-2 (CAPN2). Various oligonucleotides were assessed to knockdown CAPN2. Human iPSC-derived glutamatergic neurons were treated with various oligonucleotides targeting CAPN2 or NEAT1 (positive and negative control) by gymnotic uptake for 48 hours. Cells were lysed and RNA collected. RNA was used in real time RT-qPCR to quantify fold-change of CAPN2 or NEAT1 expression. Average fold-change of CAPN2 or NEAT1 expression of biological replicates (black dots) is represented in the top panel, average CAPN2 or NEAT1 Cp value of technical replicates (black dots) is represented in the middle panel, and average RPLP0 Cp value of technical replicates (black dots) is represented in the bottom panel. The X-axis denotes oligonucleotide; the Y-axis depicts fold-change in CAPN2 or NEAT1 expression (top panel), CAPN2 or NEAT1 Cp value (middle panel), or RPLP0 Cp value (bottom panel). Dotted horizontal line indicates corresponding fold-change or Cp value from treatment with vehicle-only negative control. Error bars represent standard deviation. For NEAT1, from left to right, vehicle, positive control, and negative control. For calpain-2 (CAPN2), from left to right, vehicle, positive control of NEAT1, negative control, and various assessed oligonucleotides.

FIG. 3. Various provided oligonucleotides display no obvious cytotoxicity as compared to vehicle treatment. Various oligonucleotides were confirmed to not significantly change percentage of live cells. Human iPSC-derived glutamatergic neurons were treated with various oligonucleotides by gymnotic uptake for 48 hours. Following this incubation period, cells were harvested and underwent Hoechst staining (5 μg/mL). The number of live, dead, and total (live+dead) cells were counted and the percent of live cells was calculated. Average live cell percentage of biological replicates is represented in the top panel, and average live cell count of biological replicates is represented in the bottom panel. The X-axis denotes oligonucleotide; the Y-axis depicts live cells as percentage of total cells (top panel) or total live cell count (bottom panel). Dotted horizontal line indicates corresponding live cell percentage or total live cell count for treatment with vehicle-only. Error bars represent standard deviation.

FIG. 4. Provided oligonucleotides can reduce levels of calpain-2 (CAPN2) mRNA. Provided oligonucleotides were demonstrated to knockdown CAPN2 in a dose-dependent manner. Human iPSC-derived spinal motor neurons were treated with indicated oligonucleotides targeting CAPN2 or negative control (targeting TUG1) at various concentrations (e.g., 20, 6.329, 2.003, 0.634, 0.201, 0.063, 0.020, 0.006 μM) by gymnotic uptake for 72 hours. Cells were lysed and RNA collected. RNA was used in real time RT-qPCR to quantify CAPN2 knockdown. The X-axis denotes concentration of oligonucleotide; the Y-axis denotes % knockdown of calpain-2 (CAPN2). Each data point represents mean of replicate treatments, except the data points for oligonucleotide 39 at 6.329 μM and oligonucleotides 14 and 39 at 0.201 μM which represent a single replicate. Error bars represent standard deviation.

FIG. 5. Provided oligonucleotides can provide neuroprotective effects. Provided oligonucleotides were demonstrated to reduce NF-L excretion following exposure to toxicity triggers (e.g., vincristine, rotenone, or colchicine). Human iPSC-derived spinal motor neurons were treated with indicated oligonucleotide targeting CAPN2 or oligonucleotide targeting TUG1 at 20 μM by gymnotic uptake for 48 hours. At 48 hours after initial treatment with oligonucleotides, neurons were treated again with indicated oligonucleotides at 20 μM by gymnotic uptake and various toxicity triggers (e.g., vincristine (at 1.5 nM, 3 nM, or 6 nM), rotenone (at 5 μM, 10 μM, or 15 μM), colchicine (at 10 nM, 100 nM, or 1000 nM)) for 24 hours. At 24 hours after exposure to the toxicity triggers, supernatant was collected and assayed for NF-L using a commercially available kit. The X-axis denotes treatment (e.g., vehicle (TE buffer), negative control oligonucleotide targeting TUG1, oligonucleotide composition 14); the Y-axis depicts relative concentration of NF-L levels following exposure to toxic triggers (e.g., vincristine (A), rotenone (B), colchicine (C)) normalized to vehicle control. Mean values are represented by bars with technical replicates (N=4) represented by black dots. Error bars represent standard deviation. Statistical analysis was performed using one-way ANOVA with Dunnett's (vehicle vs. TUG1/ASO14), and unpaired t-test (TUG vs. ASO14); *=p<0.05, **=p<0.005, ***=p<0.0005, ns=not significant.

FIG. 6. Provided oligonucleotides can provide knockdown of calpain-2 (CAPN2) mRNA. Human iPSC-derived motor neurons were treated with various oligonucleotide compositions targeting CAPN2 or TUG1 (negative control) by gymnotic uptake for 48 hours. After 48 hours, media was refreshed to remove oligonucleotides. Cells were lysed at various time points (0, 3, 7, 10, 14, 21 days) following removal of oligonucleotides and RNA collected. RNA was used in RT-qPCR to quantify CAPN2 expression. % knockdown of CAPN2 was determined as compared to mean negative control % knockdown of CAPN2 for the corresponding time point. Mean values are represented by bars and replicates (N=3) are indicated by a dot or triangle. The X-axis denotes days (D0, D3, D7, D10, D14, D21) after removal of oligonucleotides; the Y-axis depicts % knockdown of CAPN2. Error bars represent standard deviation.

FIG. 7. Provided oligonucleotides can provide knockdown of calpain-2 protein. Human iPSC-derived motor neurons were treated with oligonucleotide compositions targeting calpain-2 (CAPN2) or TUG1 (negative control) by gymnotic uptake for 48 hours. After 48 hours, media was refreshed to remove oligonucleotides. Cells were lysed at various time points (0, 3, 7, 10, 14, 21 days) following removal of oligonucleotides. Lysates were used in western blotting to quantify calpain-2 protein levels. Shown are data for oligonucleotide 14 (A) and oligonucleotide 39 (B). Mean values are represented by bars and replicates (N=2) are indicated by a dot or triangle. The X-axis denotes days (D0, D3, D7, D10, D14, D21) after removal of oligonucleotides; the Y-axis depicts relative knockdown of calpain-2 protein (% over mean negative control calpain-2 protein level at corresponding time point). Error bars represent standard deviation.

FIG. 8. Provided oligonucleotides display no significant effect on cell morphology as compared to vehicle treatment. Human iPSC-derived motor neurons were treated with oligonucleotide compositions targeting calpain-2 (CAPN2) or TUG1 (negative control) by gymnotic uptake for 48 hours. After 48 hours, media was refreshed to remove oligonucleotides. Cell morphology was assessed by brightfield imaging at various time points (0, 3, 21 days) following removal of oligonucleotides. Representative images are shown. Time point (D0, D3, D21) for each row of images is indicated by labels at left; treatment (vehicle (TE buffer), negative control oligonucleotide targeting TUG1, oligonucleotide composition 14, oligonucleotide composition 39) for each column of images is indicated by labels at top.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”: (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more: (v) the terms “about” and “approximately” may be understood to permit standard variation as would be understood by those of ordinary skill in the art: and (vi) where ranges are provided, endpoints are included.

Unless otherwise specified, description of oligonucleotides and elements thereof (e.g., base sequence, sugar modifications, internucleotidic linkages, linkage phosphorus stereochemistry, patterns thereof, etc.) is from 5′ to 3′. As those skilled in the art will appreciate, in some embodiments, oligonucleotides may be provided and/or utilized as various forms, e.g., salt forms, particularly pharmaceutically acceptable salt forms, e.g., sodium salts. As those skilled in the art will also appreciate, in some embodiments, individual oligonucleotides within a composition may be considered to be of the same constitution and/or structure even though, within such composition (e.g., a liquid composition), particular such oligonucleotides might be in different forms, e.g., salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H⁺) are of the same constitution and/or structure, such individual oligonucleotides may properly be considered to be of the same constitution and/or structure.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynl groups and hybrids thereof such as (cycloalkyl)alkyl, (cycloalkenyl)alkyl or (cycloalkyl)alkenyl.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a nucleic acid is one that contains a number of, in some embodiments, a continuous stretch of, nucleobases that are characteristic of that nucleic acid.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon: charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Identity: As used herein, the term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In some embodiments, an internucleotidic linkage is a modified internucleotidic linkage (not a natural phosphate linkage). In some embodiments, an internucleotidic linkage is a “modified internucleotidic linkage” wherein at least one oxygen atom or —OH of a phosphodiester linkage is replaced by a different organic or inorganic moiety. In some embodiments, such an organic or inorganic moiety is selected from ═S, ═Se, ═NR′, —SR′, —SeR′, —N(R′)₂, B(R′)₃, —S—, —Se—, and —N(R′)—, wherein each R^{s} is independently —H or an optionally substituted group selected from C₁₋₁₀ aliphatic, C₆₋₁₄ aryl, C₁₋₁₀ heteroaliphatic having 1-5 heteroatoms, 5-10membered heteroaryl having 1-5 heteroatoms and 3-10 membered heterocyclyl having 1-4 heteroatoms, or two or more R^{s} groups are taken together with their intervening atoms to from an optionally substituted 3-10 membered ring having 0-5 heteroatoms in addition to the intervening atoms. In some embodiments, a modified internucleotidic linkage is a phosphorothioate linkage. In some embodiments, an internucleotidic linkage is one of, e.g., PNA (peptide nucleic acid) or PMO (phosphorodiamidate Morpholino oligomer) linkage. It is understood by a person of ordinary skill in the art that an internucleotidic linkage may exist as an anion or cation at a given pH due to the existence of acid or base moieties in the linkage.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is chiral (e.g., as in phosphorothioate internucleotidic linkages). In some embodiments, a linkage phosphorus atom is achiral (e.g., as in natural phosphate linkages).

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In some embodiments, as described in the present disclosure, a modified sugar is substituted ribose or deoxyribose. In some embodiments, a modified sugar comprises a 2′-modification. Examples of useful 2′-modification are widely utilized in the art and described herein. In some embodiments, a 2′-modification is 2′-F. In some embodiments, a 2′-modification is 2′-OR, wherein R is optionally substituted C₁₋₁₀ aliphatic. In some embodiments, a 2′-modification is 2′-OMe. In some embodiments, a 2′-modification is 2′-MOE. In some embodiments, a modified sugar is a bicyclic sugar (e.g., a sugar used in LNA, BNA, etc.). In some embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases: nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a naturally-occurring nucleobases are modified adenine, guanine, uracil, cytosine, or thymine. In some embodiments, a naturally-occurring nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In some embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, UI adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, an oligonucleotide is from about 9 to about 39 nucleosides in length. In some embodiments, an oligonucleotide is from about 25 to about 70 nucleosides in length. In some embodiments, an oligonucleotide is from about 26 to about 70 nucleosides in length. In some embodiments, an oligonucleotide is from about 27 to about 70 nucleosides in length. In some embodiments, an oligonucleotide is from about 28 to about 70 nucleosides in length. In some embodiments, an oligonucleotide is from about 29 to about 70 nucleosides in length. In some embodiments, an oligonucleotide is from about 30 to about 70 nucleosides in length. In some embodiments, an oligonucleotide is from about 31 to about 70 nucleosides in length. In some embodiments, an oligonucleotide is from about 32 to about 70 nucleosides in length. In some embodiments, an oligonucleotide is from about 25 to about 60 nucleosides in length. In some embodiments, an oligonucleotide is from about 25 to about 50 nucleosides in length. In some embodiments, an oligonucleotide is from about 25 to about 40 nucleosides in length. In some embodiments, an oligonucleotide is from about 30 to about 40 nucleosides in length. In some embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In some embodiments, an oligonucleotide is at least 4 nucleosides in length. In some embodiments, an oligonucleotide is at least 5 nucleosides in length. In some embodiments, an oligonucleotide is at least 6 nucleosides in length. In some embodiments, an oligonucleotide is at least 7 nucleosides in length. In some embodiments, an oligonucleotide is at least 8 nucleosides in length. In some embodiments, an oligonucleotide is at least 9 nucleosides in length. In some embodiments, an oligonucleotide is at least 10 nucleosides in length. In some embodiments, an oligonucleotide is at least 11 nucleosides in length. In some embodiments, an oligonucleotide is at least 12 nucleosides in length. In some embodiments, an oligonucleotide is at least 15 nucleosides in length. In some embodiments, an oligonucleotide is at least 15 nucleosides in length. In some embodiments, an oligonucleotide is at least 16 nucleosides in length. In some embodiments, an oligonucleotide is at least 17 nucleosides in length. In some embodiments, an oligonucleotide is at least 18 nucleosides in length. In some embodiments, an oligonucleotide is at least 19 nucleosides in length. In some embodiments, an oligonucleotide is at least 20 nucleosides in length. In some embodiments, an oligonucleotide is at least 25 nucleosides in length. In some embodiments, an oligonucleotide is at least 26 nucleosides in length. In some embodiments, an oligonucleotide is at least 27 nucleosides in length. In some embodiments, an oligonucleotide is at least 28 nucleosides in length. In some embodiments, an oligonucleotide is at least 29 nucleosides in length. In some embodiments, an oligonucleotide is at least 30 nucleosides in length. In some embodiments, an oligonucleotide is at least 31 nucleosides in length. In some embodiments, an oligonucleotide is at least 32 nucleosides in length. In some embodiments, an oligonucleotide is at least 33 nucleosides in length. In some embodiments, an oligonucleotide is at least 34 nucleosides in length. In some embodiments, an oligonucleotide is at least 35 nucleosides in length. In some embodiments, an oligonucleotide is at least 36 nucleosides in length. In some embodiments, an oligonucleotide is at least 37 nucleosides in length. In some embodiments, an oligonucleotide is at least 38 nucleosides in length. In some embodiments, an oligonucleotide is at least 39 nucleosides in length. In some embodiments, an oligonucleotide is at least 40 nucleosides in length. In some embodiments, an oligonucleotide is 25 nucleosides in length. In some embodiments, an oligonucleotide is 26 nucleosides in length. In some embodiments, an oligonucleotide is 27 nucleosides in length. In some embodiments, an oligonucleotide is 28 nucleosides in length. In some embodiments, an oligonucleotide is 29 nucleosides in length. In some embodiments, an oligonucleotide is 30 nucleosides in length. In some embodiments, an oligonucleotide is 31 nucleosides in length. In some embodiments, an oligonucleotide is 32 nucleosides in length. In some embodiments, an oligonucleotide is 33 nucleosides in length. In some embodiments, an oligonucleotide is 34 nucleosides in length. In some embodiments, an oligonucleotide is 35 nucleosides in length. In some embodiments, an oligonucleotide is 36 nucleosides in length. In some embodiments, an oligonucleotide is 37 nucleosides in length. In some embodiments, an oligonucleotide is 38 nucleosides in length. In some embodiments, an oligonucleotide is 39 nucleosides in length. In some embodiments, an oligonucleotide is 40 nucleosides in length. In some embodiments, each nucleoside counted in an oligonucleotide length independently comprises a nucleobase comprising a ring having at least one nitrogen ring atom. In some embodiments, each nucleoside counted in an oligonucleotide length independently comprises A, T, C, G, or U, or optionally substituted A, T, C, G, or U, or an optionally substituted tautomer of A, T, C, G or U.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₄-Ph which may be substituted with R^{∘}; —CH═CHPh, which may be substituted with R^{∘}; —(CH₂)₀₋₄O(CH₂)₀₋₄-pyridyl which may be substituted with R^{∘}; —NO₂; —CN; —N₃; —(CH₂)₀₋₄N(R^{∘})₂; —(CH₂)N(R^{∘})C(O)R^{∘}; —N(R^{∘})C(S)R^{∘}; —(CH₂)₀₋₄N(R^{∘})C(O)NR^{∘}₂: —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}₂; —(CH₂)₀₋₄C(O)OR^{∘}; —(CH₂)₀₋₄C(O)SR; —(CH₂)₀₋₄C(O)OSiR^{∘}₂; —(CH₂)₀₋₄OC(O)R^{∘}, —OC(O)(CH₂)₀₋₄SR^{∘}, —SC(S)SR^{∘}, —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄—OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃,]; —P(OR^{∘})₂[B(R^{∘})₃]; —OP(R^{∘})₂[B(R^{∘})₃]; —OP(OR^{∘})₂[B(R^{∘})₃]; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR_{●}, —(CH₂)₀₋₂SH, —(CH₂)₀₋₂NH₂, —(CH₂)₀₋₂NHR^{●}, —(CH₂)_{0- 2}NR^{●}₂, —NO₂, —SiR^{●}₃, —OSiR^{●}, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}, wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₆ aliphatic, —CH₂Ph, —O(CH₂)⁰⁻¹Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin, tale; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O— and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Protecting group: The term “protecting group,” as used herein, is well known in the art and includes those described in detail in Protecting Groups in Organic Synthesis, T. W. Greene and P. G. M. Wuts, 3^{rd} edition. John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate. 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), V-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4′-tris(levulinoyloxyphenyl)methyl, 4,4′,4″-tris(benzoyloxyphenyl)methyl, 3-(imidazol-1-yl)bis(4′,4′-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative. α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifluoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound (e.g., an oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In some embodiments, a sugar is a RNA or DNA sugar (ribose or deoxyribose). In some embodiments, a sugar is a modified ribose or deoxyribose sugar, e.g., 2′-modified, 5′-modified, etc. As described herein, in some embodiments, when used in oligonucleotides and/or nucleic acids, modified sugars may provide one or more desired properties, activities, etc. In some embodiments, a sugar is optionally substituted ribose or deoxyribose. In some embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., oligonucleotides) generally also apply to pharmaceutically acceptable salts of such compounds.

As used in the present disclosure, in some embodiments, “one or more” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In some embodiments, “one or more” is one. In some embodiments, “one or more” is two. In some embodiments, “one or more” is three. In some embodiments, “one or more” is four. In some embodiments, “one or more” is five. In some embodiments, “one or more” is six. In some embodiments, “one or more” is seven. In some embodiments, “one or more” is eight. In some embodiments, “one or more” is nine. In some embodiments, “one or more” is ten. In some embodiments, “one or more” is at least one. In some embodiments, “one or more” is at least two. In some embodiments, “one or more” is at least three. In some embodiments, “one or more” is at least four. In some embodiments, “one or more” is at least five. In some embodiments, “one or more” is at least six. In some embodiments, “one or more” is at least seven. In some embodiments, “one or more” is at least eight. In some embodiments, “one or more” is at least nine. In some embodiments, “one or more” is at least ten.

As used in the present disclosure, in some embodiments, “at least one” is “one or more” as described herein.

### DESCRIPTION OF CERTAIN EMBODIMENTS

Among other things, the present disclosure provides various oligonucleotides and compositions thereof. In some embodiments, oligonucleotides of the present disclosure target calpain-2 and can hybridize with a calpain-2 transcript, e.g., a calpain-2 mRNA. In some embodiments, provided technologies. e.g., oligonucleotide, compositions, methods, etc., reduce levels of calpain-2 transcripts and/or products thereof. Use of naturally occurring nucleic acids is limited, for example, by their susceptibility to endo- and exo-nucleases. As such, various synthetic counterparts have been developed to circumvent these shortcomings and/or to further improve various properties and activities. In some embodiments, provided oligonucleotides comprise various chemical modifications, e.g., nucleobase modifications, sugar modifications, internucleotidic linkage modifications, etc., which, among other things, render these molecules less susceptible to degradation and improve other properties and/or activities. In some embodiments, an oligonucleotide comprises one or more features described herein, e.g., base sequence, length, wing, core, activity, etc. In some embodiments, an oligonucleotide has a base sequence described herein, and/or a wing-core-wing structure as described herein.

### Base Sequences

Base sequences of various oligonucleotides are of sufficient lengths so that they can form duplexes with complementary sequences in target nucleic acids for one or more biological functions. In some embodiments, oligonucleotides specifically target their target nucleic acids. In some embodiments, the base sequence of a provided oligonucleotide is or comprises a sequence that is complementary to a portion in a target nucleic acid (a “target portion”), e.g., a calpain-2 gene or a transcript thereof. In some embodiments, such a sequence complementary to a target portion is about 10, 11, 12, 13, 14, 15, 16, 17, 18, 19, 20, 21, 22, 23, 24, 25 or more nucleobases in length. In some embodiments, a target portion is or comprises a characteristic portion of a nucleic acid sequence (e.g., of a calpain-2 gene or a transcript thereof) which characteristic portion defines the nucleic acid sequence over others in a relevant organism; for example, the characteristic portion is not in other genomic nucleic acid sequences (e.g., genes) or transcripts thereof in a relevant organism (e.g., for human calpain-2, its characteristic portion is not in other human nucleic acid sequences or transcripts thereof). In some embodiments, a characteristic portion of a transcript defines that transcript over other transcripts in a relevant organism; for example, in some embodiments, the characteristic portion is not in transcripts that are transcribed from a different nucleic acid sequence (e.g., a different gene). In some embodiments, transcript variants from a nucleic acid sequence (e.g., mRNA variants of a gene) may share a common characteristic portion that defines them from transcripts of other nucleic acids, e.g., transcripts of other genes. In some embodiments, a characteristic portion in a transcript defines the transcript from other transcript(s) of the same nucleic acid sequence (e.g., a gene) and/or other alleles of the nucleic acid sequence. In some embodiments, a characteristic portion defines a particular allele (and/or transcripts thereof) over other allele(s) (and/or transcripts thereof). In some embodiments, a characteristic portion comprises sequences that are separated in a nucleic acid. In some embodiments, a characteristic portion is a contiguous stretch of nucleobases in a nucleic acid (a “characteristic sequence”). A characteristic portion or sequence may have various numbers of nucleobases. In some embodiments, there are about 10, 11, 12, 13, 14, 15, 16, 17, 18, 19, 20, 21, 22, 23, 24, 25 or more nucleobases in a characteristic portion or sequence; in some embodiments, there are about 10; in some embodiments, there are about 11; in some embodiments, there are about 12; in some embodiments, there are about 13: in some embodiments, there are about 14: in some embodiments, there are about 15; in some embodiments, there are about 16; in some embodiments, there are about 17: in some embodiments, there are about 18; in some embodiments, there are about 19; in some embodiments, there are about 20; in some embodiments, there are about 21; in some embodiments, there are about 22; in some embodiments, there are about 23; in some embodiments, there are about 24; in some embodiments, there are about 25; in some embodiments, there are about 25 or more.

In some embodiments, an oligonucleotide comprises a sequence that is identical or complementary to a characteristic portion of a nucleic acid. In some embodiments, an oligonucleotide comprises a sequence that is identical or complementary to a characteristic portion of a calpain-2 transcript. In some embodiments, an oligonucleotide comprises a sequence that is complementary to a characteristic portion of a calpain-2 transcript. In some embodiments, the base sequence of an oligonucleotide is identical or complementary to a characteristic portion of a nucleic acid. In some embodiments, the base sequence of an oligonucleotide is identical or complementary to a characteristic portion of a calpain-2 transcript. In some embodiments, the base sequence of an oligonucleotide is complementary to a characteristic portion of a calpain-2 transcript. In some embodiments, a characteristic portion is a characteristic sequence.

In some embodiments, a characteristic sequence of a calpain-2 transcript is or comprises a complementary sequence of the sequence of an oligonucleotide in Table 1. In some embodiments, a characteristic sequence is or comprises GGAAGCCUACAGAAACUGAU (SEQ ID NO: 15), wherein each U may be independently replaced with T. In some embodiments, a characteristic sequence is or comprises AUCAACUGAACCAGUAUGCC (SEQ ID NO: 16), wherein each U may be independently replaced with T. In some embodiments, a characteristic sequence is or comprises AACCACUGCCUGACCUGAGC (SEQ ID NO: 17), wherein each U may be independently replaced with T. In some embodiments, a characteristic sequence is or comprises AGCUCUGCAAAAAGGCUCUC (SEQ ID NO: 18), wherein each U may be independently replaced with T. In some embodiments, a characteristic sequence is or comprises ACUAGAGGCACAGAGCUGGA (SEQ ID NO: 19), wherein each U may be independently replaced with T. In some embodiments, a characteristic sequence is or comprises ACAACUGCCCAAGCUGGAAC (SEQ ID NO: 20), wherein each U may be independently replaced with T. In some embodiments, a characteristic sequence is or comprises CAGCCAAGGACUAAGCUUCC (SEQ ID NO: 21), wherein each U may be independently replaced with T.

In some embodiments, an oligonucleotide can hybridize to a region of a nucleic acid. In some embodiments, oligonucleotides that can specifically hybridize (e.g., through sequence complementarity) to certain region(s) of a nucleic acid can more effectively reduce levels of the nucleic acid than oligonucleotides that specifically hybridize (e.g., through sequence complementarity) to one or more reference regions of the nucleic acid. In some embodiments, a region has a length of about 20-200 (e.g., about 20-150, 20-100, 30-200, 30-150, 40-200, 40-150, 50-100, or about 20, 25, 30, 35, 40, 50, 60, 70, 80, 90, 100, 110, 120, 130, 140, 150, 160, 170, 180, 190 or 200) nucleobases. In some embodiments, a region has a length of about 30 nucleobases. In some embodiments, a region has a length of about 40 nucleobases. In some embodiments, a region has a length of about 50 nucleobases. In some embodiments, a region has a length of about 60 nucleobases. In some embodiments, a region has a length of about 70 nucleobases. In some embodiments, a region has a length of about 80 nucleobases. In some embodiments, a region has a length of about 90 nucleobases. In some embodiments, a region has a length of about 100 nucleobases. In some embodiments, a region has a length of about 120 nucleobases. In some embodiments, a region has a length of about 150 nucleobases. In some embodiments, a region has a length of about 200 nucleobases. In some embodiments, a region comprises a base sequence of an oligonucleotide in Table 1, which in some embodiments, is in the middle of a region. For example, in some embodiments, a region is or comprises GGAAGCCUACAGAAACUGAU (SEQ ID NO: 22). In some embodiments, a region is or comprises AUCAACUGAACCAGUAUGCC (SEQ ID NO: 23). In some embodiments, a region is or comprises AACCACUGCCUGACCUGAGC (SEQ ID NO: 24). In some embodiments, a region is or comprises AGCUCUGCAAAAAGGCUCUC (SEQ ID NO: 25). In some embodiments, a region is or comprises ACUAGAGGCACAGAGCUGGA (SEQ ID NO: 26). In some embodiments, a region is or comprises ACAACUGCCCAAGCUGGAAC (SEQ ID NO: 27). In some embodiments, a region is or comprises CAGCCAAGGACUAAGCUUCC (SEQ ID NO: 28). In some embodiments, a reference region is or comprises UCCCUCACCUUGAAUGAAGA (SEQ ID NO: 29).

In some embodiments, base sequences of oligonucleotides comprise or consist of about 10-50 (e.g., about 15-50, 16-50, 17-50, 18-50, 19-50, 20-50, 15-30, 20-30, 15-25 or 20-25, or at least about 10, 11, 12, 13, 14, 15, 16, 17, 18, 19, 20, 21, 22, 23, 24, 25, 30, 35, 40, 45: in some embodiments, at least about 15; in some embodiments, at least about 16: in some embodiments, at least about 17; in some embodiments, at least about 18; in some embodiments, at least about 19; in some embodiments, at least about 20; in some embodiments, at least about 21; in some embodiments, at least about 22; in some embodiments, at least about 23; in some embodiments, at least about 24; in some embodiments, at least about 25) contiguous bases that are identical to or complementary to a base sequence of equal length in a calpain-2 gene or a transcript (e.g., mRNA) thereof. In some embodiments, they ac complementary to a base sequence of equal length in a calpain-2 transcript.

In certain embodiments, a base sequence of an oligonucleotide is at least about 50%, 60%, 70%, 75%, 80%, 85%, 90%, 91%, 92%, 93%, 94%, 95%, 96%, 97%, 98%, or 99%, or 100% complementary to a target sequence in a calpain-2 transcript. In some embodiments, a base sequence of an oligonucleotide is fully complementary to a target sequence in a calpain-2 transcript.

In some embodiments, the base sequence of an oligonucleotide has about 80% or more identity with the base sequence of an oligonucleotide disclosed in Table 1, wherein each T can be independently replaced with U and vice versa. In some embodiments, the base sequence of an oligonucleotide has about 85% or more identity with the base sequence of an oligonucleotide disclosed in Table 1, wherein each T can be independently replaced with U and vice versa. In some embodiments, the base sequence of an oligonucleotide has about 90% or more identity with the base sequence of an oligonucleotide disclosed in Table 1, wherein each T can be independently replaced with U and vice versa. In some embodiments, the base sequence of an oligonucleotide has about 95% or more identity with the base sequence of an oligonucleotide disclosed in Table 1, wherein each T can be independently replaced with U and vice versa.

In some embodiments, the base sequence of an oligonucleotide comprises a continuous span of about 15 or more bases of an oligonucleotide disclosed in a Table 1, wherein each T can be independently replaced with U and vice versa. In some embodiments, the base sequence of an oligonucleotide comprises a continuous span of about 16 or more bases of an oligonucleotide disclosed in a Table 1, wherein each T can be independently replaced with U and vice versa. In some embodiments, the base sequence of an oligonucleotide comprises a continuous span of about 17 or more bases of an oligonucleotide disclosed in a Table 1, wherein each T can be independently replaced with U and vice versa. In some embodiments, the base sequence of an oligonucleotide comprises a continuous span of about 18 or more bases of an oligonucleotide disclosed in a Table 1, wherein each T can be independently replaced with U and vice versa. In some embodiments, the base sequence of an oligonucleotide comprises a continuous span of about 19 or more bases of an oligonucleotide disclosed in a Table 1, wherein each T can be independently replaced with U and vice versa. In some embodiments, the base sequence of an oligonucleotide comprises a continuous span of about 20 or more bases of an oligonucleotide disclosed in a Table 1, wherein each T can be independently replaced with U and vice versa.

In some embodiments, the base sequence of an oligonucleotide comprises the base sequence of an oligonucleotide in Table 1, wherein each T may be independently replaced with U and vice versa. In some embodiments, the base sequence of an oligonucleotide is the base sequence of an oligonucleotide in Table 1, wherein each T may be independently replaced with U and vice versa. For example, in some embodiments, the base sequence of an oligonucleotide comprises ATCAGTTTCTGTAGGCTTCC (SEQ ID NO: 1), wherein each T may be independently replaced with U. In some embodiments, the base sequence of an oligonucleotide comprises GGCATACTGGTTCAGTTGAT (SEQ ID NO: 2), wherein each T may be independently replaced with U. In some embodiments, the base sequence of an oligonucleotide comprises GCTCAGGTCAGGCAGTGGTT (SEQ ID NO: 3), wherein each T may be independently replaced with U. In some embodiments, the base sequence of an oligonucleotide comprises GAGAGCCTTTTTGCAGAGCT (SEQ ID NO: 4) wherein each T may be independently replaced with U. In some embodiments, the base sequence of an oligonucleotide comprises TCCAGCTCTGTGCCTCTAGT (SEQ ID NO: 5), wherein each T may be independently replaced with U. In some embodiments, the base sequence of an oligonucleotide comprises GTTCCAGCTTGGGCAGTTGT (SEQ ID NO: 64, wherein each T may be independently replaced with U. In some embodiments, the base sequence of an oligonucleotide comprises GGAAGCTTAGTCCTTGGCTG (SEQ ID NO: 7), wherein each T may be independently replaced with U. In some embodiments, the base sequence of an oligonucleotide is ATCAGTTTCTGTAGGCTTCC (SEQ ID NO: 1), wherein each T may be independently replaced with U. In some embodiments, the base sequence of an oligonucleotide is GGCATACTGGTTCAGTTGAT (SEQ ID NO: 2), wherein each T may be independently replaced with U. In some embodiments, the base sequence of an oligonucleotide is GCTCAGGTCAGGCAGTGGTT (SEQ ID NO; 3), wherein each T may be independently replaced with U. In some embodiments, the base sequence of an oligonucleotide is GAGAGCCTTTTTGCAGAGCT (SEQ ID NO: 4), wherein each T may be independently replaced with U. In some embodiments, the base sequence of an oligonucleotide is TCCAGCTCTGTGCCTCTAGT (SEQ ID NO: 5), wherein each T may be independently replaced with U. In some embodiments, the base sequence of an oligonucleotide is GTTCCAGCTTGGGCAGTTGT (SEQ ID NO: 6), wherein each T may be independently replaced with U. In some embodiments, the base sequence of an oligonucleotide is GGAAGCTTAGTCCTTGGCTG (SEQ ID NO: 7, wherein each T may be independently replaced with U.

In some embodiments, the base sequence of an oligonucleotide comprises ATCAGTTTCTGTAGGCTTCC (SEQ ID NO: 8). In some embodiments, the base sequence of an oligonucleotide comprises GGCATACTGGTTCAGTTGAT (SEQ ID NO: 9). In some embodiments, the base sequence of an oligonucleotide comprises GCTCAGGTCAGGCAGTGGTT (SEQ ID NO: 10). In some embodiments, the base sequence of an oligonucleotide comprises GAGAGCCTTTTTGCAGAGCT (SEQ ID NO: 11). In some embodiments, the base sequence of an oligonucleotide comprises TCCAGCTCTGTGCCTCTAGT (SEQ ID NO: 12). In some embodiments, the base sequence of an oligonucleotide comprises GTTCCAGCTTGGGCAGTTGT (SEQ ID NO: 13). In some embodiments, the base sequence of an oligonucleotide comprises GGAAGCTTAGTCCTTGGCTG (SEQ ID NO: 14). In some embodiments, the base sequence of an oligonucleotide is ATCAGTTTCTGTAGGCTTCC (SEQ ID NO: 8). In some embodiments, the base sequence of an oligonucleotide is GGCATACTGGTTCAGTTGAT (SEQ ID NO: 9). In some embodiments, the base sequence of an oligonucleotide is GCTCAGGTCAGGCAGTGGTT (SEQ ID NO: 10). In some embodiments, the base sequence of an oligonucleotide is GAGAGCCTTTTTGCAGAGCT (SEQ ID NO: 11). In some embodiments, the base sequence of an oligonucleotide is TCCAGCTCTGTGCCTCTAGT (SEQ ID NO: 12). In some embodiments, the base sequence of an oligonucleotide is GTTCCAGCTTGGGCAGTTGT (SEQ ID NO: 13). In some embodiments, the base sequence of an oligonucleotide is GGAAGCTTAGTCCTTGGCTG (SEQ ID NO: 14).

### Lengths

As appreciated by those skilled in the art, oligonucleotides can be of various lengths to provide desired properties and/or activities for various uses. Many technologies for assessing, selecting and/or optimizing oligonucleotide length are available in the art and can be utilized in accordance with the present disclosure. As demonstrated herein, in many embodiments, provided oligonucleotides are of suitable lengths to hybridize with their targets and reduce levels of their targets and/or a product thereof. In some embodiments, an oligonucleotide is long enough to recognize a target nucleic acid (e.g., a calpain-2 mRNA). In some embodiments, an oligonucleotide is sufficiently long to distinguish between a target nucleic acid and other nucleic acids (e.g., a nucleic acid having a base sequence which is not a calpain-2 sequence) to reduce off-target effects. In some embodiments, an oligonucleotide is sufficiently short to reduce complexity of manufacture or production and to reduce cost of products.

In some embodiments, the base sequence of an oligonucleotide is about 10-100 nucleobases in length. In some embodiments, a base sequence is about 10-50 nucleobases in length. In some embodiments, a base sequence is about 15-50 nucleobases in length. In some embodiments, a base sequence is about 15-30 nucleobases in length. In some embodiments, a base sequence is about 15-25 nucleobases in length. In some embodiments, a base sequence is about 15-22 nucleobases in length. In some embodiments, a base sequence is about 18-22 nucleobases in length. In some embodiments, a base sequence is about 10, 11, 12, 13, 14, 15, 16, 17, 18, 19, 20, 21, 22, 23, 24, or 25 nucleobases in length. In some embodiments, a base sequence is at least about 12 nucleobases in length. In some embodiments, a base sequence is at least about 13 nucleobases in length. In some embodiments, a base sequence is at least about 14 nucleobases in length. In some embodiments, a base sequence is at least about 15 nucleobases in length. In some embodiments, a base sequence is at least about 16 nucleobases in length. In some embodiments, a base sequence is at least about 17 nucleobases in length. In some embodiments, a base sequence is at least about 18 nucleobases in length. In some embodiments, a base sequence is at least about 19 nucleobases in length. In some embodiments, a base sequence is at least about 20 nucleobases in length. In some embodiments, a base sequence is at least about 21 nucleobases in length. In some embodiments, a base sequence is at least about 22 nucleobases in length. In some embodiments, a base sequence is at least about 23 nucleobases in length. In some embodiments, a base sequence is at least about 24 nucleobases in length. In some embodiments, a base sequence is at least about 25 nucleobases in length. In some embodiments, a base sequence is about 15 nucleobases in length. In some embodiments, a base sequence is about 16 nucleobases in length. In some embodiments, a base sequence is about 17 nucleobases in length. In some embodiments, a base sequence is about 18 nucleobases in length. In some embodiments, a base sequence is about 19 nucleobases in length. In some embodiments, a base sequence is about 20 nucleobases in length. In some embodiments, a base sequence is about 21 nucleobases in length. In some embodiments, a base sequence is about 22 nucleobases in length. In some embodiments, a base sequence is about 23 nucleobases in length. In some embodiments, a base sequence is about 24 nucleobases in length. In some embodiments, a base sequence is about 25 nucleobases in length. In some other embodiments, a base sequence is about at least about 30 nucleobases in length. In some embodiments, each nucleobase independently comprises an optionally substituted monocyclic, bicyclic or polycyclic ring wherein at least one ring atom is nitrogen. In some embodiments, each nucleobase is independently optionally substituted adenine, cytosine, guanosine, thymine, or uracil, or an optionally substituted tautomer of adenine, cytosine, guanosine, thymine, or uracil.

### Nucleobases

Various nucleobases may be utilized in provided oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5mC, 5-hydroxymethyl C, etc. In some embodiments, a nucleobase is alkyl-substituted A, T, C, G or U. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5mC. In some embodiments, a nucleobase is substituted A, T, C, G or U. In some embodiments, a nucleobase is a substituted tautomer of A, T, C, G or U. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5mC may be treated the same as C [e.g., an oligonucleotide having 5mC in place of C (e.g., AT5mCG) is considered to have the same base sequence as an oligonucleotide having C at the corresponding location(s) (e.g., ATCG)].

In some embodiments, an oligonucleotide comprises one or more A, T, C, G or U. In some embodiments, an oligonucleotide comprises one or more optionally substituted A, T, C, G or U. In some embodiments, an oligonucleotide comprises one or more 5-methylcytosine (5mC), 5-hydroxymethylcytosine, 5-formylcytosine, or 5-carboxylcytosine. In some embodiments, an oligonucleotide comprises one or more 5mC. In some embodiments, each nucleobase in an oligonucleotide is independently selected from optionally substituted A, T, C, G and U, and optionally substituted tautomers of A, T, C, G and U. In some embodiments, each nucleobase in an oligonucleotide is independently optionally protected A, T, C, 5mC, G and U. In some embodiments, each nucleobase in an oligonucleotide is optionally substituted A, T, C, G or U. In some embodiments, each nucleobase in an oligonucleotide is selected from the group consisting of A, T, C, G, U, and 5mC.

In some embodiments, a nucleobase is optionally substituted 2AP or DAP. In some embodiments, a nucleobase is optionally substituted 2AP. In some embodiments, a nucleobase is optionally substituted DAP. In some embodiments, a nucleobase is 2AP. In some embodiments, a nucleobase is DAP.

In some embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, RNA, 2003, 9, 1034-1048, Limbach et al. Nucleic Acids Research, 1994, 22, 2183-2196 and Revankar and Rao, Comprehensive Natural Products Chemistry, vol. 7, 313.

In some embodiments, a provided oligonucleotide comprises one or more 5-methylcytosine. In some embodiments, the present disclosure provides an oligonucleotide whose base sequence is disclosed herein, e.g., in Table 1, wherein each T may be independently replaced with U and vice versa, and each cytosine is optionally and independently replaced with 5-methylcytosine or vice versa. As appreciated by those skilled in the art, in some embodiments, 5mC may be treated as C with respect to base sequence of an oligonucleotide—such oligonucleotide comprises a nucleobase modification at the C position (e.g., see various oligonucleotides in Table 1 or A2). In description of oligonucleotides, unless otherwise noted, nucleobases, sugars and internucleotidic linkages are non-modified.

In some embodiments, a modified nucleobase is a modified nucleobase known in the art, e.g., WO2017/210647. In some embodiments, modified nucleobases are expanded-size nucleobases in which one or more aryl and/or heteroaryl rings, such as phenyl rings, have been added.

Nucleobases may be protected during oligonucleotide synthesis. Various protection technologies are available and can be utilized in accordance with the present disclosure.

In some embodiments, a modified nucleobase is 5-substituted pyrimidines, 6-azapyrimidines, alkyl or alkynyl substituted pyrimidines, alkyl substituted purines, or N-2, N-6 and O-6 substituted purines. In certain embodiments, a modified nucleobase is selected form 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. In some embodiments, a modified nucleobases is a tricyclic pyrimidine, such as 1,3-diazaphenoxazine-2-one, 1,3-diazaphenothiazine-2-one or 9-(2-aminoethoxy)-1,3-diazaphenoxazine-2-one (G-clamp). In some embodiments, a modified nucleobases is one in which a purine or pyrimidine base is replaced with other heterocycles, for example, 7-deaza-adenine, 7-deazaguanosine, 2-aminopyridine or 2-pyridone.

In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One example of a universal base is 3-nitropyrrole.

In some embodiments, nucleosides that can be utilized in provided technologies comprise modified nucleobases and/or modified sugars, e.g., 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine: 5-carboxylcytosine; N-methyladenosine: 7-methylguanosine; 5-methylaminoethyluridine; 5-methoxyaminomethyl-2-thiouridine; beta,D-mannosylqueosine; 5-methoxycarbonylmethyluridine: 5-methoxyuridine: 2-methylthio-N⁶-isopentenyladenosine: N-((9-beta,D-ribofuranosyl-2-methylthiopurine-6-yl)carbamoyl)threonine; N-((9-beta,D-ribofuranosylpurine-6-yl)-N-methylcarbamoyl)threonine: uridine-5-oxyacetic acid methylester; uridine-5-oxyacetic acid (v); pseudouridine; queosine; 2-thiocytidine: 5-methyl-2-thiouridine; 2-thiouridine; 4-thiouridine; 5-methyluridine; 2′-O-methyl-5-methyluridine; and 2′-O-methyluridine.

In some embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5-iodouracil, or 2,6-diaminopurine. In some embodiments, a nucleobase comprises substitution with a fluorescent or biomolecule binding moiety. In some embodiments, a substituent is a fluorescent moiety. In some embodiments, a substituent is biotin or avidin.

In some embodiments, a nucleobase is one described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the nucleobase of each of which is incorporated herein by reference.

### Sugars

Various sugars, including modified sugars, can be utilized in accordance with the present disclosure. In some embodiments, the present disclosure provides sugar modifications and patterns thereof optionally in combination with other structural elements (e.g., nucleobase modifications and patterns thereof, internucleotidic linkage modifications and patterns thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar, e.g., various sugars in many oligonucleotides in Table 1 (unless otherwise notes), is a natural DNA sugar (in DNA nucleic acids or oligonucleotides, having the structure of

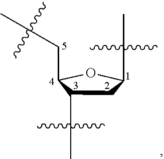

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5-end group (e.g., —OH), and if at the 3′-end of an oligonucleotide, the 3′ position may be connected to a 3′-end group (e.g., —OH). In some embodiments, a sugar is a natural RNA sugar (in RNA nucleic acids or oligonucleotides, having the structure of

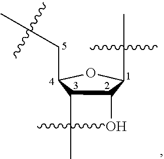

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3-end group (e.g., —OH). In some embodiments, a sugar is a modified sugar in that it is not a natural DNA sugar or a natural RNA sugar. Among other things, modified sugars may provide improved stability and/or affinity. In some embodiments, modified sugars can be utilized to alter and/or optimize one or more hybridization characteristics. In some embodiments, modified sugars can be utilized to alter and/or optimize target recognition. In some embodiments, modified sugars can be utilized to optimize Tm. In some embodiments, modified sugars can be utilized to improve oligonucleotide activities.

Sugars can be bonded to internucleotidic linkages at various positions. As non-limiting examples, internucleotidic linkages can be bonded to the 2′, 3′, 4′ or 5′ positions of sugars. As most commonly in natural nucleic acids, an internucleotidic linkage typically connects with one sugar at the 5′ position and another sugar at the 3′ position unless otherwise indicated.

In some embodiments, a sugar is an optionally substituted natural DNA or RNA sugar. In some embodiments, a sugar is optionally substituted

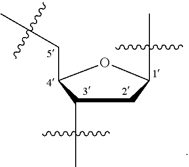 [SMILES: *C[C@H]1O[C@@H](*)C[C@H]1*]

In some embodiments, the 2′ position is optionally substituted. In some embodiments, a sugar is

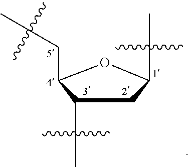 [SMILES: *C[C@H]1O[C@@H](*)C[C@H]1*]

In some embodiments, a sugar has the structure of

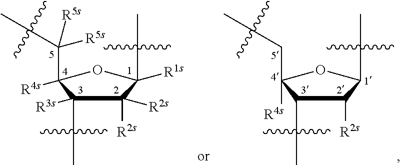

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357, the substituents, sugar modifications, descriptions of R^{1s}, R^{2s}, R^{3s}, R^{4s}, and R^{5s}, and modified sugars of each of which are independently incorporated herein by reference). In some embodiments, each of R^{1s}, R^{2s}, R^{3s}, R^{4s}, and R^{5s} is independently —F, —Cl, —Br, —I, —CN, —N³, —NO, —NO₂, -L^{s}-R′, -L^{s}-OR′, -L^{s}-SR′, -L^{s}-N(R′)₂, —O-L-OR′, —O-L^{s}-SR′, or —O-L^{s}-N(R′)₂, wherein each R′ is independently —H or an optionally substituted group selected from C₁₋₁₀ aliphatic, C₆₋₁₄ aryl, C₁₋₁₀ heteroaliphatic having 1-5 heteroatoms, 5-10 membered heteroaryl having 1-5 heteroatoms and 3-10 membered heterocyclyl having 1-4 heteroatoms, or two or more R′ groups are taken together with their intervening atoms to from an optionally substituted 3-10 membered ring having 0-5 heteroatoms in addition to the intervening atoms, independently as described herein, and L^{s} is a covalent bond or optionally substituted bivalent C₁₋₆ aliphatic or heteroaliphatic having 1-4 heteroatoms. In some embodiments, a sugar has the structure of

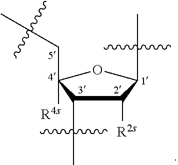

In some embodiments, R^{4s} is —H. In some embodiments, a sugar has the structure of

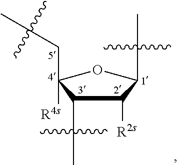

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R² is —OMe. In some embodiments, R^{2s} is —OCH₂CH₂OMe.

In some embodiments, a sugar has the structure of

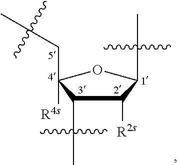

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C2-O—CH₂—C4. In some embodiments, L^{s} is C2-O—CH₂—C4. In some embodiments, L^{s} is C2-O—(R)—CH(CH₂CH₃)—C4. In some embodiments, L^{s} is C2-O—(S)—CH(CH₂CH₃)—C4.

In some embodiments, a modified sugar contains one or more substituents at the 2′ position ((typically one substituent, and often at the axial position or R^{2s}) independently selected from —F; —CF₃, —CN, —N₃, —NO, —NO₂, —OR′, —SR′, or —N(R′)₂, wherein each R^{s} is independently described in the present disclosure, and in some embodiments, optionally substituted C₁₋₁₀ aliphatic; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀alkyl)₂, —NH—(C₁-C₁₀ alkylene)-O—(C₁-C₁₀alkyl), or —N(C₁-C₁₀ alkyl-(C₁-C₁₀ alkylene)-O—(C₁-C₁₀ alkyl), wherein each of the alkyl, alkylene, alkenyl and alkynyl is independently and optionally substituted. In some embodiments, a substituent is —O(CH₂)ₙOCH₃, —O(CH₂)ₙNH₂, MOE, DMAOE, or DMAEOE, wherein n is from 1 to about 10.

In some embodiments, a modified sugar is a natural RNA sugar whose 2′-OH is replaced with a group selected from —F, —CF₃, —CN, —N₃, —NO, —NO₂, —OR′, —SR′, or —N(R′)₂, wherein each R′ is independently described in the present disclosure: —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₁-C₁₀ alkenyl). —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

In some embodiments, a sugar modification is a 2′-modification. In some embodiments, a 2′-modification is a 2′-OR^{s} modification. In some embodiments, R^{s} is optionally substituted C₁₋₄ aliphatic. In some embodiments, R^{s} is optionally substituted C₁₋₆ alkyl. In some embodiments, a modification is 2′-OMe. In some embodiments, a modification is 2′-MOE. In some embodiments, a 2′-modification is S-cEt. In some embodiments, a modified sugar is an LNA sugar. In some embodiments, a 2′-modification is —F.

In some embodiments, a sugar modification replaces a sugar moiety with another cyclic or acyclic moiety. Examples of such moieties are widely known in the art, e.g., those in morpholino, glycol nucleic acids, PNA, etc., and may be utilized in accordance with the present disclosure.

In some embodiments, one or more sugars of an oligonucleotide are independently modified. In some embodiments, each sugar of an oligonucleotide or a portion thereof (e.g., a wing) is independently modified. In some embodiments, a modified sugar comprises a 2′-modification. In some embodiments, each modified sugar independently comprises a 2′-modification. In some embodiments, a 2′-modification is 2′-OR^{s}, wherein R^{s} is optionally substituted C₁₋₆ aliphatic. In some embodiments, a 2′-modification is a 2′-OMe modification. In some embodiments, a 2′-modification is a 2′-MOE modification. In some embodiments, a 2′-modification is an LNA sugar modification. In some embodiments, a 2′-modification is 2′-F. In some embodiments, each sugar modification is independently a 2′-modification. In some embodiments, each sugar modification is independently a 2′-OR^{s} modification. In some embodiments, each sugar modification is independently 2′-OR^{s}, wherein R^{s} is optionally substituted C₁₋₆ alkyl. In some embodiments, each sugar modification is 2′-OMe. In some embodiments, each sugar modification is 2′-MOE. In some embodiments, each sugar modification is independently 2′-OMe or 2′-MOE. In some embodiments, each sugar modification is independently 2′-OMe, 2′-MOE, or a LNA sugar.

As those skilled in the art will appreciate, modifications of sugars, nucleobases, internucleotidic linkages, etc. can and are often utilized in combination in oligonucleotides, e.g., see various oligonucleotides in Table 1.

In some embodiments, a sugar is one described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the sugars of each of which is incorporated herein by reference.

Various additional sugars useful for preparing oligonucleotides or analogs thereof are known in the art and may be utilized in accordance with the present disclosure.

### Internucleotidic Linkages

In some embodiments, oligonucleotides comprise base modifications, sugar modifications, and/or internucleotidic linkage modifications. Various internucleotidic linkages can be utilized in accordance with the present disclosure to link units comprising nucleobases, e.g., nucleosides. In some embodiments, oligonucleotides comprise both one or more modified internucleotidic linkages and one or more natural phosphate linkages. As widely known by those skilled in the art, natural phosphate linkages are widely found in natural DNA and RNA molecules, they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O—)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S—)O—.

In some embodiments, an oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate.

In some embodiments, an internucleotidic linkage is described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951. WO 2019/075357, WO 2019/200185, WO 2019/217784, and/or WO 2019/032612, the internucleotidic linkages of each of which is incorporated herein by reference. In some embodiments, an internucleotidic linkage is described in U.S. Pat. Nos. 3,687,808, 4,469,863, 4,476,301, 5,177,195, 5,023,243, 5,034,506, 5,166,315, 5,185,444, 5,188,897, 5,214,134, 5,216,141, 5,235,033, 5,264,423, 5,264,564, 5,276,019, 5,278,302, 5,286,717, 5,321,131, 5,399,676, 5,405,938, 5,405,939, 5,434,257, 5,453,496, 5,455,233, 5,466,677, 5,466,677, 5,470,967, 5,476,925, 5,489,677, 5,519,126, 5,536,821, 5,541,307, 5,541,316, 5,550,111, 5,561,225, 5,563,253, 5,571,799, 5,587,361, 5,596,086, 5,602,240, 5,608,046, 5,610,289, 5,618,704, 5,623,070, 5,625,050, 5,633,360, 564,562, 5,663,312, 5,677,437, 5,677,439, 6,160,109, 6,239,265, 6,028,188, 6,124,445, 6,169,170, 6,172,209, 6,277,603, 6,326,199, 6,346,614, 6,444,423, 6,531,590, 6,534,639, 6,608,035, 6,683,167, 6,858,715, 6,867,294, 6,878,805, 7,015,315, 7,041,816, 7,273,933, 7,321,029, or RE39464, the internucleotidic linkages of each of which is incorporated herein by reference.

In some embodiments, an oligonucleotide comprises one or more modified internucleotidic linkages. In some embodiments, each modified internucleotidic linkage is independently a phosphorothioate internucleotidic linkage. In some embodiments, one or more, e.g., about 1-20, 1-15, 1-10, or about 1, 2, 3, 4, 5, 6, 7, 8, 9, 10, 11, 12, 13, 14, 15, 16, 17, 18, 19, 20, or about 10%, 20%, 25%, 30%, 40%, 50%, 60%, 70%, 75%, 80%, 85%, 90%, 95% or more of all internucleotidic linkages in an oligonucleotide, are independently phosphorothioate internucleotidic linkage internucleotidic linkage. In some embodiments, about 10% or more of all internucleotidic linkages are independently phosphorothioate internucleotidic linkages. In some embodiments, about 25% or more of all internucleotidic linkages are independently phosphorothioate internucleotidic linkages. In some embodiments, about 50% or more of all internucleotidic linkages are independently phosphorothioate internucleotidic linkages. In some embodiments, about 60% or more of all internucleotidic linkages are independently phosphorothioate internucleotidic linkages. In some embodiments, about 70% or more of all internucleotidic linkages are independently phosphorothioate internucleotidic linkages. In some embodiments, about 75% or more of all internucleotidic linkages are independently phosphorothioate internucleotidic linkages. In some embodiments, about 80% or more of all internucleotidic linkages are independently phosphorothioate internucleotidic linkages. In some embodiments, about 85% or more of all internucleotidic linkages are independently phosphorothioate internucleotidic linkages. In some embodiments, about 90% or more of all internucleotidic linkages are independently phosphorothioate internucleotidic linkages. In some embodiments, about 95% or more of all internucleotidic linkages are independently phosphorothioate internucleotidic linkages. In some embodiments, each internucleotidic linkage bonded to a natural DNA sugar is independently a phosphorothioate internucleotidic linkage. In some embodiments, each internucleotidic linkage in an oligonucleotide is independently a phosphorothioate internucleotidic linkage.

In some embodiments, an oligonucleotide comprises one or more natural phosphate linkages. In some embodiments, each natural phosphate linkage independently bonds to at least one modified sugar. In some embodiments, each sugar bonded to a natural phosphate linkage is independently a modified sugar. In some embodiments, each sugar bonded to a natural phosphate linkage is independently a 2′-OR^{s} modified sugar or a bicyclic sugar (e.g., a LNA sugar). In some embodiments, each sugar bonded to a natural phosphate linkage is independently a 2′-OR^{s} modified sugar. In some embodiments, each sugar bonded to a natural phosphate linkage is independently a 2′-MOE modified sugar.

### Wings and Cores

In some embodiments, an oligonucleotide comprises or consists of a 5′-wing-core-wing-3′ structure.

Wings and cores can independently be of various suitable lengths. In some embodiments, there are about 1, 2, 3, 4, 5, 6, 7, 8, 9, 10, 11, 12, 13, 14, 15, 16, 17, 18, 19, 20, 21, 22, 23, 24, 25 or more nucleobases independently in a wing or core. In some embodiments, each nucleobase independently comprises an optionally substituted monocyclic, bicyclic or polycyclic ring, which ring has at least one nitrogen ring atom; in some embodiments, each nucleobase is independently optionally substituted A, T, C, G or U, or a substituted tautomer of A, T, C, G or U. In some embodiments, the number of nucleobases in a wing is 1, 2, 3, 4, 5, 6, 7, 8, 9, or 10. In some embodiments, the number is 1 for a wing. In some embodiments, the number is 2 for a wing. In some embodiments, the number is 3 for a wing. In some embodiments, the number is 4 for a wing. In some embodiments, the number is 5 for a wing. In some embodiments, the number is 6 for a wing. In some embodiments, the number is 7 for a wing. In some embodiments, the number is 8 for a wing. In some embodiments, the number is 9 for a wing. In some embodiments, the number is 10 for a wing. In some embodiments, in a wing of a wing-core-wing structure, the two wings are of the same length. In some embodiments, the two wings are of different length. In some embodiments, the number is 1, 2, 3, 4, 5, 6, 7, 8, 9, 10, 11, 12, 13, 14, 15 or more for a core. In some embodiments, the number is about 5-15, e.g., about 5, 6, 7, 8, 9, 10, 11, 12, 13, 14, 15, for a core. In some embodiments, the number is 1 for a core. In some embodiments, the number is 2 for a core. In some embodiments, the number is 3 for a core. In some embodiments, the number is 4 for a core. In some embodiments, the number is 5 for a core. In some embodiments, the number is 6 for a core. In some embodiments, the number is 7 for a core. In some embodiments, the number is 8 for a core. In some embodiments, the number is 9 for a core. In some embodiments, the number is 10 for a core. In some embodiments, the number is 11 for a core. In some embodiments, the number is 12 for a core. In some embodiments, the number is 13 for a core. In some embodiments, the number is 14 for a core. In some embodiments, the number is 15 for a core.

In some embodiments, a wing-core-wing is described as “X—Y—Z”, where “X” represents the length of the 5′ wing (as number of nucleobases), “Y” represents the length of the core (as number of nucleobases), and “Z” represents the length of the 3′ wing (as number of nucleobases). Example embodiments of X. Y, and Z include those lengths described as numbers (e.g., above) and exemplified in oligonucleotide species (e.g., in Table 1). In some embodiments, the two wings are of the same or different lengths and/or have the same or different modifications or patterns of modifications. In some embodiments, Y is between 8 and 15. In some embodiments, X, Y or Z can each independently be 1, 2, 3, 4, 5, 6, 7, 8, 9, 10, 11, 12, 13, 14, 15, 16, 17, 18, 19, 20, 25, 30 or more. In some embodiments, each of X, Y and Z is independently 1-30. In some embodiments, X—Z—Z is 5-10-5, 5-10-4, 4-10-4, 4-10-3, 3-10-3, 2-10-2, 5-9-5, 5-9-4, 4-9-5, 5-8-5, 5-8-4, 4-8-5, 5-7-5, 4-7-5, 5-7-4, or 4-74.

In some embodiments, a wing comprises one or more sugar modifications. In some embodiments, each sugar in a wing is independently modified. In some embodiments, each wing sugar in an oligonucleotide is independently modified. In some embodiments, each modified sugar independently comprises a 2′-modification (e.g., a 2′-OR^{s} modified sugar, a LNA sugar, etc.). In some embodiments, each wing sugar is independently a 2′-OR^{s} modified sugar. In some embodiments, each sugar modification in a wing is the same. In some embodiments, a wing comprises different sugar modifications, e.g., different 2′-OR^{s} modifications. In some embodiments, 2′-OR^{s} is 2′-OMe. In some embodiments, 2′-OR^{s} is 2′-MOE. In some embodiments, each sugar in a wing is a 2′-MOE modified sugar. In some embodiments, each sugar in a wing is a 2′-OMe modified sugar. In some embodiments, a wing comprises one or more 2′-OMe modified sugars and one or more 2′-MOE modified sugars.

In some embodiments, the two wings of a wing-core-wing structure comprise different sugar modifications or patterns thereof.

In some embodiments, certain sugar modifications, e.g., 2′-MOE, provide more stability under certain conditions than other sugar modifications, e.g., 2′-OMe or natural DNA or RNA sugars.

In some embodiments, a wing comprises a bicyclic sugar. In some embodiments, a bicyclic sugar is a LNA, a cEt or a BNA sugar.

In some embodiments, one or more internucleotidic linkages bonded to a 5′-wing sugar are each independently a modified internucleotidic linkage. In some embodiments, they are each independently a phosphorothioate internucleotidic linkage. In some embodiments, each internucleotidic linkage bonded to a 5′-wing sugar is independently a modified internucleotidic linkage. In some embodiments, each such internucleotidic linkage is independently a phosphorothioate internucleotidic linkage.

In some embodiments, one or more internucleotidic linkages bonded to a 3′-wing sugar are each independently a modified internucleotidic linkage. In some embodiments, they are each independently a phosphorothioate internucleotidic linkage. In some embodiments, each internucleotidic linkage bonded to a 3′-wing sugar is independently a modified internucleotidic linkage. In some embodiments, each such internucleotidic linkage is independently a phosphorothioate internucleotidic linkage.

In some embodiments, a core comprises one or more, e.g., about 1-20, 5-20, 6-20, 7-20, 8-20, 9-20, 10-20, or 5-15, or about 1, 2, 3, 4, 5, 6, 7, 8, 9, 10, 11, 12, 13, 14, 15, 16, 17, 18, 19, 20 natural DNA sugars. In some embodiments, a core comprises 2 or more natural DNA sugars. In some embodiments, a core comprises 3 or more natural DNA sugars. In some embodiments, a core comprises 4 or more natural DNA sugars. In some embodiments, a core comprises 5 or more natural DNA sugars. In some embodiments, a core comprises 6 or more natural DNA sugars. In some embodiments, a core comprises 7 or more natural DNA sugars. In some embodiments, a core comprises 8 or more natural DNA sugars. In some embodiments, a core comprises 9 or more natural DNA sugars. In some embodiments, a core comprises 10 or more natural DNA sugars. In some embodiments, a core comprises 11 or more natural DNA sugars. In some embodiments, a core comprises 12 or more natural DNA sugars. In some embodiments, a core comprises 13 or more natural DNA sugars. In some embodiments, a core comprises 14 or more natural DNA sugars. In some embodiments, a core comprises 15 or more natural DNA sugars. In some embodiments, such DNA sugars are consecutive. In some embodiments, each sugar in a core is independently a natural DNA sugar.

In some embodiments, one or more internucleotidic linkages bonded to a core sugar are each independently a modified internucleotidic linkage. In some embodiments, they are each independently a phosphorothioate internucleotidic linkage. In some embodiments, each internucleotidic linkage bonded to a core sugar is independently a modified internucleotidic linkage. In some embodiments, each such internucleotidic linkage is independently a phosphorothioate internucleotidic linkage.

In some embodiments, a core is able to hybridize to a target mRNA, forming a duplex structure recognizable by RNase H, such that RNase H is able to cleave the mRNA.

### Oligonucleotides

Among other things, the present disclosure provides various oligonucleotides. As described herein, oligonucleotides may contain various nucleobase modifications, sugar modifications, internucleotidic linkages and patterns thereof. In some embodiments, the present disclosure provides the oligonucleotides in Table 1 as examples.

TABLE 1
Example oligonucleotides and compositions.

| Oligo- | | |
|---|---|---|
| nucleo- | Base | |
| tide | Sequence | Description |
| 1 | GGGTGGCTCCTCCAAT | /52MOErG/*/i2MOErG/*/ |
| | GATA | i2MOErG/*/i2MOErT/*/ |
| | (SEQ ID NO: 30) | i2MOErG/*G*/iMe-dC/*T*/ |
| | | iMe-dC/*/iMe-dC/*T*/ |
| | | iMe-dC/*/iMe-dC/*A*A*/ |
| | | i2MOErT/*/i2MOErG/*/ |
| | | i2MOErA/*/i2MOErT/*/ |
| | | 32MOErA/ |
| | | (SEQ ID NO: 103) |
| 2 | GGGCTCCTTGGCAGAT | /52MOErG/*/i2MOErG/*/ |
| | GTCT | i2MOErG/*/i2MOErC/*/ |
| | (SEQ ID NO: 31) | i2MOErT/*/iMe-dC/*/ |
| | | iMe-dC/*T*T*G*G*/ |
| | | iMe-dC/*A*G*A*/i2MOErT/ |
| | | */i2MOErG/*/i2MOErT/*/ |
| | | i2MOErC/*/32MOErT/ |
| | | (SEQ ID NO: 104) |
| 3 | TGCCAGCAGCCAGCAG | /52MOErT/*/i2MOErG/*/ |
| | TCAC | i2MOErC/*/i2MOErC/*/ |
| | (SEQ ID NO: 32) | i2MOErA/*G*/iMe-dC/ |
| | | *A*G*/iMe-dC/*/iMe-dC/ |
| | | *A*G*/iMe-dC/*A*/ |
| | | i2MOErG/*/i2MOErT/*/ |
| | | i2MOErC/*/i2MOErA/*/ |
| | | 32MOErC/ |
| | | (SEQ ID NO: 105) |
| 4 | GTGAGGGAGGCAATGG | /52MOErG/*/i2MOErT/*/ |
| | CTGC | i2MOErG/*/i2MOErA/*/ |
| | (SEQ ID NO: 33) | i2MOErG/*G*G*A*G*G*/ |
| | | iMe-dC/*A*A*T*G*/ |
| | | i2MOErG/*/i2MOErC/*/ |
| | | i2MOErT/*/i2MOErG/*/ |
| | | 32MOErC/ |
| | | (SEQ ID NO: 106) |
| 5 | TCTTCATTCAAGGTGA | /52MOErT/*/i2MOErC/*/ |
| | GGGA | i2MOErT/*/i2MOET/*/ |
| | (SEQ ID NO: 34) | i2MOErC/*A*T*T*/iMe-dC/ |
| | | *A*A*G*G*T*G*/i2MOErA/ |
| | | */i2MOErG/*/i2MOErG/*/ |
| | | i2MOErG/*/32MOErA/ |
| | | (SEQ ID NO: 107) |
| 6 | GAGCCAGGATTTCTTC | /52MOErG/*/i2MOErA/*/ |
| | ATTC | i2MOErG/*/i2MOErC/*/ |
| | (SEQ ID NO: 35) | i2MOErC/ |
| | | *A*G*G*A*T*T*T*/iMe-dC/ |
| | | *T*T*/i2MOErC/*/ |
| | | i2MOErA/*/i2MOErT/*/ |
| | | i2MOErT/*/32MOErC/ |
| | | (SEQ ID NO: 108) |
| 7 | TCCCTGCATAGTTTTC | /52MOErT/*/i2MOErC/*/ |
| | CTGG | i2MOErC/*/i2MOErC/*/ |
| | (SEQ ID NO: 36) | i2MOErT/*G*/iMe-dC/ |
| | | *A*T*A*G*T*T*T*T*/ |
| | | i2MOErC/*/i2MOErC/*/ |
| | | i2MOErT/*/i2MOErG/*/ |
| | | 32MOErG/ |
| | | (SEQ ID NO: 109) |
| 8 | CCACCACCTCCACCCA | /52MOErC/*/i2MOErC/*/ |
| | CTCG | i2MOErA/*/i2MOErC/*/ |
| | (SEQ ID NO: 37) | i2MOErC/*A*/iMe-dC/*/ |
| | | iMe-dC/*T*/iMe-dC/*/ |
| | | iMe-dC/*A*/iMe-dC/*/ |
| | | iMe-dC/*/iMe-dC/*/ |
| | | i2MOErA/*/i2MOErC/*/ |
| | | i2MOErT/*/i2MOErC/*/ |
| | | 32MOErG/ |
| | | (SEQ ID NO: 110) |
| 9 | GAAGCCCTCAGTGGTG | /52MOErG/*/i2MOErA/*/ |
| | GCAC | i2MOErA/*/i2MOErG/*/ |
| | (SEQ ID NO: 38) | i2MOErC/*/iMe-dC/*/ |
| | | iMe-dC/*T*/iMe-dC/ |
| | | *A*G*T*G*G*T*/i2MOErG/ |
| | | */i2MOErG/*/i2MOErC/*/ |
| | | i2MOErA/*/32MOErC/ |
| | | (SEQ ID NO: 111) |
| 10 | GGGCTTCTTCAACTCA | /52MOErG/*/i2MOErG/*/ |
| | TACC | i2MOErG/*/i2MOErC/*/ |
| | (SEQ ID NO: 39) | i2MOErT/*T*/iMe-dC/ |
| | | *T*T*/iMe-dC/*A*A*/ |
| | | iMe-dC/*T*/iMe-dC/*/ |
| | | i2MOErA/*/i2MOErT/*/ |
| | | i2MOErA/*/i2MOErC/*/ |
| | | 32MOErC/ |
| | | (SEQ ID NO: 112) |
| 11 | TCTTGAACAGGTTGGG | /52MOErT/*/i2MOErC/*/ |
| | AGGG | i2MOErT/*/i2MOErT/*/ |
| | (SEQ ID NO: 40) | i2MOErG/*A*A*/iMe-dC/ |
| | | *A*G*G*T*T*G*G*/ |
| | | i2MOErG/*/i2MOErA/*/ |
| | | i2MOErG/*/i2MOErG/*/ |
| | | 32MOErG/ |
| | | (SEQ ID NO: 113) |
| 12 | GAGAGCCTTTTTGCAG | /52MOErG/*/i2MOErA/*/ |
| | AGCT | i2MOErG/*/i2MOErA/*/ |
| | (SEQ ID NO: 11) | i2MOErG/*/iMe-dC/*/ |
| | | iMe-dC/*T*T*T*T*T*G*/ |
| | | iMe-dC/*A*/i2MOErG/*/ |
| | | i2MOErA/*/i2MOErG/*/ |
| | | i2MOErC/*/32MOErT/ |
| | | (SEQ ID NO: 114) |
| 13 | GAGCAGCCAAGGAGAG | /52MOErG/*/i2MOErA/*/ |
| | AGCC | i2MOErG/*/i2MOErC/*/ |
| | (SEQ ID NO: 41) | i2MOErA/*G*/iMe-dC/*/ |
| | | iMe-dC/*A*A*G*G*A*G*A*/ |
| | | i2MOErG/*/i2MOErA/*/ |
| | | i2MOErG/*/i2MOErC/*/ |
| | | 32MOErC/ |
| | | (SEQ ID NO: 115) |
| 14 | ATCAGTTTCTGTAGGC | /52MOErA/*/i2MOErT/*/ |
| | TTCC | i2MOErC/*/i2MOErA/*/ |
| | (SEQ ID NO: 8) | i2MOErG/*T*T*T*/iMe-dC/ |
| | | *T*G*T*A*G*G*/i2MOErC/ |
| | | */i2MOErT/*/i2MOErT/*/ |
| | | i2MOErC/*/32MOErC/ |
| | | (SEQ ID NO: 116) |
| 15 | GTTCCAGCTTGGGCAG | /52MOErG/*/i2MOErT/*/ |
| | TTGT | i2MOErT/*/i2MOErC/*/ |
| | (SEQ ID NO: 13) | i2MOErC/*A*G*/iMe-dC/ |
| | | *T*T*G*G*G*/iMe-dC/*A*/ |
| | | i2MOErG/*/i2MOErT/*/ |
| | | i2MOErT/*/i2MOErG/*/ |
| | | 32MOErT/ |
| | | (SEQ ID NO: 117) |
| 16 | GGGTCTATAGTGTTCC | /52MOErG/*/i2MOErG/*/ |
| | AGCT | i2MOErG/*/i2MOErT/*/ |
| | (SEQ ID NO: 42) | i2MOErC/ |
| | | *T*A*T*A*G*T*G*T*T*/ |
| | | iMe-dC/*/i2MOErC/*/ |
| | | i2MOErA/*/i2MOErG/*/ |
| | | i2MOErC/*/32MOErT/ |
| | | (SEQ ID NO: 118) |
| 17 | GAATTCTCCATCTTCA | /52MOErG/*/i2MOErA/*/ |
| | TGCC | i2MOErA/*/i2MOErT/*/ |
| | (SEQ ID NO: 43) | i2MOErT/*/iMe-dC/*T*/ |
| | | iMe-dC/*/iMe-dC/*A*T*/ |
| | | iMe-dC/*T*T*/iMe-dC/*/ |
| | | i2MOErA/*/i2MOErT/*/ |
| | | i2MOErG/*/i2MOErC/*/ |
| | | 32MOErC/ |
| | | (SEQ ID NO: 119) |
| 18 | GTGCCTCAGGAAGTCA | /52MOErG/*/i2MOErT/*/ |
| | CTGA | i2MOErG/*/i2MOErC/*/ |
| | (SEQ ID NO: 44) | i2MOErC/*T*/iMe-dC/ |
| | | *A*G*G*A*A*G*T*/iMe-dC/ |
| | | */i2MOErA/*/i2MOErC/*/ |
| | | i2MOErT/*/i2MOErG/*/ |
| | | 32MOErA/ |
| | | (SEQ ID NO: 120) |
| 19 | AGTTTCCACTTCTTGT | /52MOErA/*/i2MOErG/*/ |
| | AGGT | i2MOErT/*/i2MOErT/*/ |
| | (SEQ ID NO: 45) | i2MOErT/*/iMe-dC/*/ |
| | | iMe-dC/*A*/iMe-dC/ |
| | | *T*T*/iMe-dC/*T*T*G*/ |
| | | i2MOErT/*/i2MOErA/*/ |
| | | i2MOErG/*/i2MOErG/*/ |
| | | 32MOErT/ |
| | | (SEQ ID NO: 121) |
| 20 | CTCCAGTTCCCATCCA | /52MOErC/*/i2MOErT/*/ |
| | TTTT | i2MOErC/*/i2MOErC/*/ |
| | (SEQ ID NO: 46) | i2MOErA/*G*T*T*/iMe-dC/ |
| | | */iMe-dC/*/iMe-dC/ |
| | | *A*T*/iMe-dC/*/iMe-dC/ |
| | | */i2MOErA/*/i2MOErT/*/ |
| | | i2MOErT/*/i2MOErT/*/ |
| | | 32MOErT/ |
| | | (SEQ ID NO: 122) |
| 21 | GGTACTGAGGGTTCAT | /52MOErG/*/i2MOErG/*/ |
| | CCAG | i2MOErT/*/i2MOErA/*/ |
| | (SEQ ID NO: 47) | i2MOErC/ |
| | | *T*G*A*G*G*G*T*T*/ |
| | | iMe-dC/*A*/i2MOErT/*/ |
| | | i2MOErC/*/i2MOErC/*/ |
| | | i2MOErA/*/32MOErG/ |
| | | (SEQ ID NO: 123) |
| 22 | GTCTGCCCACTTAACT | /52MOErG/*/i2MOErT/*/ |
| | CCTC | i2MOErC/*/i2MOErT/*/ |
| | (SEQ ID NO: 48) | i2MOErG/*/iMe-dC/*/ |
| | | iMe-dC/*/iMe-dC/*A*/ |
| | | iMe-dC/*T*T*A*A*/ |
| | | iMe-dC/*/i2MOErT/*/ |
| | | i2MOErC/*/i2MOErC/*/ |
| | | i2MOErT/*/32MOErC/ |
| | | (SEQ ID NO: 124) |
| 23 | GGTGGATGTTGGTCTG | /52MOErG/*/i2MOErG/*/ |
| | CCCA | i2MOErT/*/i2MOErG/*/ |
| | (SEQ ID NO: 49) | i2MOErG/ |
| | | *A*T*G*T*T*G*G*T*/ |
| | | iMe-dC/*T*/i2MOErG/*/ |
| | | i2MOErC/*/i2MOErC/*/ |
| | | i2MOErC/*/32MOErA/ |
| | | (SEQ ID NO: 125) |
| 24 | GTTTTTGCTGAGGTGG | /52MOErG/*/i2MOErT/*/ |
| | ATGT | i2MOErT/*/i2MOErT/*/ |
| | (SEQ ID NO: 50) | i2MOErT/*T*G*/iMe-dC/ |
| | | *T*G*A*G*G*T*G*/ |
| | | i2MOErG/*/i2MOErA/*/ |
| | | i2MOErT/*/i2MOErG/*/ |
| | | 32MOErT/ |
| | | (SEQ ID NO: 126) |
| 25 | ACTCTTCAAGATTGGC | /52MOErA/*/i2MOErC/*/ |
| | CTCG | i2MOErT/*/i2MOErC/*/ |
| | (SEQ ID NO: 51) | i2MOErT/*T*/iMe-dC/ |
| | | *A*A*G*A*T*T*G*G*/ |
| | | i2MOErC/*/i2MOErC/*/ |
| | | i2MOErT/*/i2MOErC/*/ |
| | | 32MOErG/ |
| | | (SEQ ID NO: 127) |
| 26 | CCATCATCAATGTCAT | /52MOErC/*/i2MOErC/*/ |
| | CCTC | i2MOErA/*/i2MOErT/*/ |
| | (SEQ ID NO: 52) | i2MOErC/*A*T*/iMe-dC/ |
| | | *A*A*T*G*T*/iMe-dC/*A*/ |
| | | i2MOErT/*/i2MOErC/*/ |
| | | i2MOErC/*/i2MOErT/*/ |
| | | 32MOErC/ |
| | | (SEQ ID NO: 128) |
| 27 | GTCTCCTGAATCCATC | /52MOErG/*/i2MOErT/*/ |
| | ATCA | i2MOErC/*/i2MOErT/*/ |
| | (SEQ ID NO: 53) | i2MOErC/*/iMe-dC/ |
| | | *T*G*A*A*T*/iMe-dC/*/ |
| | | iMe-dC/*A*T*/i2MOErC/*/ |
| | | i2MOErA/*/i2MOErT/*/ |
| | | i2MOErC/*/32MOErA/ |
| | | (SEQ ID NO: 129) |
| 28 | CTGGGCAAACAGTCTC | /52MOErC/*/i2MOErT/*/ |
| | CTGA | i2MOErG/*/i2MOErG/*/ |
| | (SEQ ID NO: 54) | i2MOErG/*/iMe-dC/ |
| | | *A*A*A*/iMe-dC/*A*G*T*/ |
| | | iMe-dC/*T*/i2MOErC/*/ |
| | | i2MOErC/*/i2MOErT/*/ |
| | | i2MOErG/*/32MOErA/ |
| | | (SEQ ID NO: 130) |
| 29 | TGGTCTGCAGCTCAAA | /52MOErT/*/i2MOErG/*/ |
| | GGCA | i2MOErG/*/i2MOErT/*/ |
| | (SEQ ID NO: 55) | i2MOErC/*T*G*/iMe-dC/ |
| | | *A*G*/iMe-dC/*T*/ |
| | | iMe-dC/*A*A*/i2MOErA/*/ |
| | | i2MOErG/*/i2MOErG/*/ |
| | | i2MOErC/*/32MOErA/ |
| | | (SEQ ID NO: 131) |
| 30 | GCTTTGCTAGAACCCT | 52MOErG/*/i2MOErC/*/ |
| | TCTC | i2MOErT/*/i2MOErT/*/ |
| | (SEQ ID NO: 56) | i2MOErT/*G*/iMe-dC/ |
| | | *T*A*G*A*A*/iMe-dC/*/ |
| | | iMe-dC/*/iMe-dC/*/ |
| | | i2MOErT/*/i2MOErT/*/ |
| | | i2MOErC/*/i2MOErT/*/ |
| | | 32MOErC/ |
| | | (SEQ ID NO: 132) |
| 31 | ATGTAGAACTCCTTCA | /52MOErA/*/i2MOErT/*/ |
| | GCCC | i2MOErG/*/i2MOErT/*/ |
| | (SEQ ID NO: 57) | i2MOErA/*G*A*A*/iMe-dC/ |
| | | *T*/iMe-dC/*/iMe-dC/ |
| | | *T*T*/iMe-dC/*/i2MOErA/ |
| | | */i2MOErG/*/i2MOErC/*/ |
| | | i2MOErC/*/32MOErC/ |
| | | (SEQ ID NO: 133) |
| 32 | GGGCATCTTGAAACCT | /52MOErG/*/i2MOErG/*/ |
| | GCTT | i2MOErG/*/i2MOErC/*/ |
| | (SEQ ID NO: 58) | i2MOErA/*T*/iMe-dC/ |
| | | *T*T*G*A*A*A*/iMe-dC/*/ |
| | | iMe-dC/*/i2MOErT/*/ |
| | | i2MOErG/*/i2MOErC/*/ |
| | | i2MOErT/*/32MOErT/ |
| | | (SEQ ID NO: 134) |
| 33 | GGTGGAGTTGACAGGG | /52MOErG/*/i2MOErG/*/ |
| | CATC | i2MOErT/*/i2MOErG/*/ |
| | (SEQ ID NO: 59) | i2MOErG/*A*G*T*T*G*A*/ |
| | | iMe-dC/*A*G*G*/i2MOErG/ |
| | | */i2MOErC/*/i2MOErA/*/ |
| | | i2MOErT/*/32MOErC/ |
| | | (SEQ ID NO: 135) |
| 34 | GAGCTCTATTGTTCCA | /52MOErG/*/i2MOErA/*/ |
| | GTAT | i2MOErG/*/i2MOErC/*/ |
| | (SEQ ID NO: 60) | i2MOErT/*/iMe-dC/ |
| | | *T*A*T*T*G*T*T*/iMe-dC/ |
| | | */iMe-dC/*/i2MOErA/*/ |
| | | i2MOErG/*/i2MOErT/*/ |
| | | i2MOErA/*/32MOErT/ |
| | | (SEQ ID NO: 136) |
| 35 | AGTCTTCAGGCAGATT | /52MOErA/*/i2MOErG/*/ |
| | AGTT | i2MOErT/*/i2MOErC/*/ |
| | (SEQ ID NO: 61) | i2MOErT/*T*/iMe-dC/ |
| | | *A*G*G*/iMe-dC/ |
| | | *A*G*A*T*/i2MOErT/*/ |
| | | i2MOErA/*/i2MOErG/*/ |
| | | i2MOErT/*/32MOErT/ |
| | | (SEQ ID NO: 137) |
| 36 | GGAAGCTTAGTCCTTG | /52MOErG/*/i2MOErG/*/ |
| | GCTG | i2MOErA/*/i2MOErA/*/ |
| | (SEQ ID NO: 14) | i2MOErG/*/iMe-dC/ |
| | | *T*T*A*G*T*/iMe-dC/*/ |
| | | iMe-dC/*T*T*/i2MOErG/*/ |
| | | i2MOErG/*/i2MOErC/*/ |
| | | i2MOErT/*/32MOErG/ |
| | | (SEQ ID NO: 138) |
| 37 | GCTGTTAAATACCTCC | /52MOErG/*/i2MOErC/*/ |
| | CTGC | i2MOErT/*/i2MOErG/*/ |
| | (SEQ ID NO: 62) | i2MOErT/*T*A*A*A*T*A*/ |
| | | iMe-dC/*/iMe-dC/*T*/ |
| | | iMe-dC/*/i2MOErC/*/ |
| | | i2MOErC/*/i2MOErT/*/ |
| | | i2MOErG/*/32MOErC/ |
| | | (SEQ ID NO: 139) |
| 38 | TCCCTTTGGTCTTTGT | (52MOErT/*/i2MOErC/*/ |
| | AAGA | i2MOErC/*/i2MOErC/*/ |
| | (SEQ ID NO: 63) | i2MOErT/*T*T*G*G*T*/ |
| | | iMe-dC/*T*T*T*G*/ |
| | | i2MOErT/*/i2MOErA/*/ |
| | | i2MOErA/*/i2MOErG/*/ |
| | | 32MOErA/ |
| | | (SEQ ID NO: 140) |
| 39 | GGCATACTGGTTCAGT | /52MOErG/*/i2MOErG/*/ |
| | TGAT | i2MOErC/*/i2MOErA/*/ |
| | (SEQ ID NO: 9) | i2MOErT/*A*/iMe-dC/ |
| | | *T*G*G*T*T*/iMe-dC/ |
| | | *A*G*/i2MOErT/*/i2MOErT/ |
| | | */i2MOErG/*/i2MOErA/*/ |
| | | 32MOErT/ |
| | | (SEQ ID NO: 141) |
| 40 | ACTAGTGGTGGTGAAG | /52MOErA/*/i2MOErC/*/ |
| | TTCA | i2MOErT/*/i2MOErA/*/ |
| | (SEQ ID NO: 64) | i2MOErG/ |
| | | *T*G*G*T*G*G*T*G*A*A*/ |
| | | i2MOErG/*/i2MOErT/*/ |
| | | i2MOErT/*/i2MOErC/*/ |
| | | 32MOErA/ |
| | | (SEQ ID NO: 142) |
| 41 | GGAGCCACTCTTGCCA | /52MOErG/*/i2MOErG/*/ |
| | GTGT | i2MOErA/*/i2MOErG/*/ |
| | (SEQ ID NO: 65) | i2MOErC/*/iMe-dC/*A*/ |
| | | iMe-dC/*T*/iMe-dC/ |
| | | *T*T*G*/iMe-dC/*/ |
| | | iMe-dC/*/i2MOErA/*/ |
| | | i2MOErG/*/i2MOErT/*/ |
| | | i2MOErG/*/32MOErT/ |
| | | (SEQ ID NO: 143) |
| 42 | TCCCTTCATGCCTCTG | /52MOErT/*/i2MOErC/*/ |
| | CCTT | i2MOErC/*/i2MOErC/*/ |
| | (SEQ ID NO: 66) | i2MOErT/*T*/iMe-dC/ |
| | | *A*T*G*/iMe-dC/*/ |
| | | iMe-dC/*T*/iMe-dC/*T*/ |
| | | i2MOErG/*/i2MOErC/*/ |
| | | i2MOErC/*/i2MOErT/*/ |
| | | 32MOErT/ |
| | | (SEQ ID NO: 144) |
| 43 | GTCCCTCATCTCTTCC | /52MOErG/*/i2MOErT/*/ |
| | ACCC | i2MOErC/*/i2MOErC/*/ |
| | (SEQ ID NO: 67) | i2MOErC/*T*/iMe-dC/ |
| | | *A*T*/iMe-dC/*T*/ |
| | | iMe-dC/*T*T*/iMe-dC/*/ |
| | | i2MOErC/*/i2MOErA/*/ |
| | | i2MOErC/*/i2MOErC/*/ |
| | | 32MOErC/ |
| | | (SEQ ID NO: 145) |
| 44 | GGCCTCTGTTCCTGGT | /52MOErG/*/i2MOErG/*/ |
| | GCTT | i2MOErC/*/i2MOErC/*/ |
| | (SEQ ID NO: 68) | i2MOErT/*/iMe-dC/ |
| | | *T*G*T*T*/iMe-dC/*/ |
| | | iMe-dC/*T*G*G*/i2MOErT/ |
| | | */i2MOErG/*/i2MOErC/*/ |
| | | i2MOErT/*/32MOErT/ |
| | | (SEQ ID NO: 146) |
| 45 | GCAGGGTCAGCCAGTG | /52MOErG/*/i2MOErC/*/ |
| | GAGG | i2MOErA/*/i2MOErG/*/ |
| | (SEQ ID NO: 69) | i2MOErG/*G*T*/iMe-dC/ |
| | | *A*G*/iMe-dC/*/iMe-dC/ |
| | | *A*G*T*/i2MOErG/*/ |
| | | i2MOErG/*/i2MOErA/*/ |
| | | i2MOErG/*/32MOErG/ |
| | | (SEQ ID NO: 147) |
| 46 | GTCCCAGGCCTTTCCT | /52MOErG/*/i2MOErT/*/ |
| | TTCC | i2MOErC/*/i2MOErC/*/ |
| | (SEQ ID NO: 70) | i2MOErC/*A*G*G*/iMe-dC/ |
| | | */iMe-dC/*T*T*T*/ |
| | | iMe-dC/*/iMe-dC/*/ |
| | | i2MOErT/*/i2MOErT/*/ |
| | | i2MOErT/*/i2MOErC/*/ |
| | | 32MOErC/ |
| | | (SEQ ID NO: 148) |
| 47 | TCCTTTGCTGTGTTCC | /52MOErT/*/i2MOErC/*/ |
| | TCCC | i2MOErC/*/i2MOErT/*/ |
| | (SEQ ID NO: 71) | i2MOErT/*T*G*/iMe-dC/ |
| | | *T*G*T*G*T*T*/iMe-dC/*/ |
| | | i2MOErC/*/i2MOErT/*/ |
| | | i2MOErC/*/i2MOErC/*/ |
| | | 32MOErC/ |
| | | (SEQ ID NO: 149) |
| 48 | GGCCCTTCTCCTCCTT | /52MOErG/*/i2MOErG/*/ |
| | TGCT | i2MOErC/*/i2MOErC/*/ |
| | (SEQ ID NO: 72) | i2MOErC/*T*T*/iMe-dC/ |
| | | *T*/iMe-dC/*/iMe-dC/ |
| | | *T*/iMe-dC/*/iMe-dC/ |
| | | *T*/i2MOErT/*/i2MOErT/ |
| | | */i2MOErG/*/i2MOErC/*/ |
| | | 32MOErT/ |
| | | (SEQ ID NO: 150) |
| 49 | CCAGTGTCAGGCCACC | /52MOErC/*/i2MOErC/*/ |
| | CAGC | i2MOErA/*/i2MOErG/*/ |
| | (SEQ ID NO: 73) | i2MOErT/*G*T*/iMe-dC/ |
| | | *A*G*G*/iMe-dC/*/ |
| | | iMe-dC/*A*/iMe-dC/*/ |
| | | i2MOErC/*/i2MOErC/*/ |
| | | i2MOErA/*/i2MOErG/*/ |
| | | 32MOErC/ |
| | | (SEQ ID NO: 151) |
| 50 | TCCTTCCAACTCTCCC | /52MOErT/*/i2MOErC/*/ |
| | TCCT | i2MOErC/*/i2MOErT/*/ |
| | (SEQ ID NO: 74) | i2MOErT/*/iMe-dC/*/ |
| | | iMe-dC/*A*A*/iMe-dC/ |
| | | *T*/iMe-dC/*T*/iMe-dC/ |
| | | */iMe-dC/*/i2MOErC/*/ |
| | | i2MOErT/*/i2MOErC/*/ |
| | | i2MOErC/*/32MOErT/ |
| | | (SEQ ID NO: 152) |
| 51 | CCTTCTGCACCAGACC | /52MOErC/*/i2MOErC/*/ |
| | CTCC | i2MOErT/*/i2MOErT/*/ |
| | (SEQ ID NO: 75) | i2MOErC/*T*G*/iMe-dC/ |
| | | *A*/iMe-dC/*/iMe-dC/ |
| | | *A*G*A*/iMe-dC/*/ |
| | | i2MOErC/*/i2MOErC/*/ |
| | | i2MOErT/*/i2MOErC/*/ |
| | | 32MOErC/ |
| | | (SEQ ID NO: 153) |
| 52 | GTTGGTGCATTCCTGG | /52MOErG/*/i2MOErT/*/ |
| | CTCC | i2MOErT/*/i2MOErG/*/ |
| | (SEQ ID NO: 76) | i2MOErG/*T*G*/iMe-dC/ |
| | | *A*T*T*/iMe-dC/*/ |
| | | iMe-dC/*T*G*/i2MOErG/ |
| | | */i2MOErC/*/i2MOErT/*/ |
| | | i2MOErC/*/32MOErC/ |
| | | (SEQ ID NO: 154) |
| 53 | GCCTCCACATCCCTCA | /52MOErG/*/i2MOErC/*/ |
| | CTCC | i2MOErC/*/i2MOErT/*/ |
| | (SEQ ID NO: 77) | i2MOErC/*/iMe-dC/*A*/ |
| | | iMe-dC/*A*T*/iMe-dC/*/ |
| | | iMe-dC/*/iMe-dC/*T*/ |
| | | iMe-dC/*/i2MOErA/*/ |
| | | i2MOErC/*/i2MOErT/*/ |
| | | i2MOErC/*/32MOErC/ |
| | | (SEQ ID NO: 155) |
| 54 | GCAGGTTGGTGCTGGC | /52MOErG/*/i2MOErC/*/ |
| | TGTC | i2MOErA/*/i2MOErG/*/ |
| | (SEQ ID NO: 78) | i2MOErG/*T*T*G*G*T*G*/ |
| | | iMe-dC/*T*G*G*/i2MOErC/ |
| | | */i2MOErT/*/i2MOErG/*/ |
| | | i2MOErT/*/32MOErC/ |
| | | (SEQ ID NO: 156) |
| 55 | GTGACAGGCCCAGCCC | 52MOErG/*/i2MOErT/*/ |
| | ATCC | i2MOErG/*/i2MOErA/*/ |
| | (SEQ ID NO: 79) | i2MOErC/*A*G*G*/iMe-dC/ |
| | | */iMe-dC/*/iMe-dC/ |
| | | *A*G*/iMe-dC/*/iMe-dC/ |
| | | */i2MOErC/*/i2MOErA/*/ |
| | | i2MOErT/*/i2MOErC/*/ |
| | | 32MOErC/ |
| | | (SEQ ID NO: 157) |
| 56 | CCCAGCCATGCTTCTG | /52MOErC/*/i2MOErC/*/ |
| | TCCT | i2MOErC/*/i2MOErA/*/ |
| | (SEQ ID NO: 80) | i2MOErG/*/iMe-dC/*/ |
| | | iMe-dC/*A*T*G*/iMe-dC/ |
| | | *T*T*/iMe-dC/*T*/ |
| | | i2MOErG/*/i2MOErT/*/ |
| | | i2MOErC/*/i2MOErC/*/ |
| | | 32MOErT/ |
| | | (SEQ ID NO: 158) |
| 57 | GCCTCAGTCTGTCCAG | /52MOErG/*/i2MOErC/*/ |
| | CACC | i2MOErC/*/i2MOErT/*/ |
| | (SEQ ID NO: 81) | i2MOErC/*A*G*T*/iMe-dC/ |
| | | *T*G*T*/iMe-dC/*/ |
| | | iMe-dC/*A*/i2MOErG/*/ |
| | | i2MOErC/*/i2MOErA/*/ |
| | | i2MOErC/*/32MOErC/ |
| | | (SEQ ID NO: 159) |
| 58 | GCCTGACACCTGCCTC | /52MOErG/*/i2MOErC/*/ |
| | AGTC | i2MOErC/*/i2MOErT/*/ |
| | (SEQ ID NO: 82) | i2MOErG/*A*/iMe-dC/*A*/ |
| | | iMe-dC/*/iMe-dC/*T*G*/ |
| | | iMe-dC/*/iMe-dC/*T*/ |
| | | i2MOErC/*/i2MOErA/*/ |
| | | i2MOErG/*/i2MOErT/*/ |
| | | 32MOErC/ |
| | | (SEQ ID NO: 160) |
| 59 | GGTGCCAGTGCTAGGT | /52MOErG/*/i2MOErG/*/ |
| | TCTC | i2MOErT/*/i2MOErG/*/ |
| | (SEQ ID NO: 83) | i2MOErC/*/iMe-dC/ |
| | | *A*G*T*G*/iMe-dC/ |
| | | *T*A*G*G*/i2MOErT/*/ |
| | | i2MOErT/*/i2MOErC/*/ |
| | | i2MOErT/*/32MOErC/ |
| | | (SEQ ID NO: 161) |
| 60 | GCCTGGTCCCTCTCTG | /52MOErG/*/i2MOErC/*/ |
| | AGCC | i2MOErC/*/i2MOErT/*/ |
| | (SEQ ID NO: 84) | i2MOErG/*G*T*/iMe-dC/*/ |
| | | iMe-dC/*/iMe-dC/*T*/ |
| | | iMe-dC/*T*/iMe-dC/*T*/ |
| | | i2MOErG/*/i2MOErA/*/ |
| | | i2MOErG/*/i2MOErC/*/ |
| | | 32MOErC/ |
| | | (SEQ ID NO: 162) |
| 61 | AGCAGCCAGCAGTCAC | /52MOErA/*/i2MOErG/*/ |
| | CTGT | i2MOErC/*/i2MOErA/*/ |
| | (SEQ ID NO: 85) | i2MOErG/*/iMe-dC/*/ |
| | | iMe-dC/*A*G*/iMe-dC/ |
| | | *A*G*T*/iMe-dC/*A*/ |
| | | i2MOErC/*/i2MOErC/*/ |
| | | i2MOErT/*/i2MOErG/*/ |
| | | 32MOErT/ |
| | | (SEQ ID NO: 163) |
| 62 | GGAGCAGTGGGTTCCC | /52MOErG/*/i2MOErG/*/ |
| | TTGG | i2MOErA/*/i2MOErG/*/ |
| | (SEQ ID NO: 86) | i2MOErC/ |
| | | *A*G*T*G*G*G*T*T*/ |
| | | iMe-dC/*/iMe-dC/*/ |
| | | i2MOErC/*/i2MOErT/*/ |
| | | i2MOErT/*/i2MOErG/*/ |
| | | 32MOErG/ |
| | | (SEQ ID NO: 164) |
| 63 | GGCTGGAGGCACCATC | /52MOErG/*/i2MOErG/*/ |
| | ACCC | i2MOErC/*/i2MOErT/*/ |
| | (SEQ ID NO: 87) | i2MOErG/*G*A*G*G*/ |
| | | iMe-dC/*A*/iMe-dC/*/ |
| | | iMe-dC/*A*T*/i2MOErC/*/ |
| | | i2MOErA/*/i2MOErC/*/ |
| | | i2MOErC/*/32MOErC/ |
| | | (SEQ ID NO: 165) |
| 64 | GCTCTGTGGCCCTGGT | /52MOErG/*/i2MOErC/*/ |
| | ATGC | i2MOErT/*/i2MOErC/*/ |
| | (SEQ ID NO: 88) | i2MOErT/*G*T*G*G*/ |
| | | iMe-dC/*/iMe-dC/*/ |
| | | iMe-dC/*T*G*G*/i2MOErT/ |
| | | */i2MOErA/*/i2MOErT/*/ |
| | | i2MOErG/*/32MOErC/ |
| | | (SEQ ID NO: 166) |
| 65 | CCTTGCCCTGCTCCTG | /52MOErC/*/i2MOErC/*/ |
| | ATGT | i2MOErT/*/i2MOErT/*/ |
| | (SEQ ID NO: 89) | i2MOErG/*/iMe-dC/*/ |
| | | iMe-dC/*/iMe-dC/*T*G*/ |
| | | iMe-dC/*T*/iMe-dC/*/ |
| | | iMc-dC/*T*/i2MOErG/*/ |
| | | i2MOErA/*/i2MOErT/*/ |
| | | i2MOErG/*/32MOErT/ |
| | | (SEQ ID NO: 167) |
| 66 | GTCCCTTGTCCTCTCA | /52MOErG/*/i2MOErT/*/ |
| | GGCC | i2MOErC/*/i2MOErC/*/ |
| | (SEQ ID NO: 90) | i2MOErC/*T*T*G*T*/ |
| | | iMe-dC/*/iMe-dC/*T*/ |
| | | iMe-dC/*T*/iMe-dC/*/ |
| | | i2MOErA/*/i2MOErG/*/ |
| | | i2MOErG/*/i2MOErC/*/ |
| | | 32MOErC/ |
| | | (SEQ ID NO: 168) |
| 67 | GCAGTTCTCTCTCTGG | /52MOErG/*/i2MOErC/*/ |
| | CTCC | i2MOErA/*/i2MOErG/*/ |
| | (SEQ ID NO: 91) | i2MOErT/*T*/iMe-dC/*T*/ |
| | | iMe-dC/*T*/iMe-dC/*T*/ |
| | | iMe-dC/*T*G*/i2MOErG/*/ |
| | | i2MOErC/*/i2MOErT/*/ |
| | | i2MOErC/*/32MOErC/ |
| | | (SEQ ID NO: 169) |
| 68 | AGGAGCCCAGCCCTGA | /52MOErA/*/i2MOErG/*/ |
| | GCAG | i2MOErG/*/i2MOErA/*/ |
| | (SEQ ID NO: 92) | i2MOErG/*/iMe-dC/*/ |
| | | iMe-dC/*/iMe-dC/*A*G*/ |
| | | iMe-dC/*/iMe-dC/*/ |
| | | iMe-dC/*T*G*/i2MOErA/*/ |
| | | i2MOErG/*/i2MOErC/*/ |
| | | i2MOErA/*/32MOErG/ |
| | | (SEQ ID NO: 170) |
| 69 | GCTCTCCACCATCCAG | /52MOErG/*/i2MOErC/*/ |
| | GCCA | i2MOErT/*/i2MOErC/*/ |
| | (SEQ ID NO: 93) | i2MOErT/*/iMe-dC/*/ |
| | | iMe-dC/*A*/iMe-dC/*/ |
| | | iMe-dC/*A*T*/iMe-dC/*/ |
| | | iMe-dC/*A*/i2MOErG/*/ |
| | | i2MOErG/*/i2MOErC/*/ |
| | | i2MOErC/*/32MOErA/ |
| | | (SEQ ID NO: 171) |
| 70 | GTTCCCTGCCTCTACC | /52MOErG/*/i2MOErT/*/ |
| | TCCC | i2MOErT/*/i2MOErC/*/ |
| | (SEQ ID NO: 94) | i2MOErC/*/iMe-dC/*T*G*/ |
| | | iMe-dC/*/iMe-dC/*T*/ |
| | | iMe-dC/*T*A*/iMe-dC/*/ |
| | | i2MOErC/*/i2MOErT/*/ |
| | | i2MOErC/*/i2MOErC/*/ |
| | | 32MOErC/ |
| | | (SEQ ID NO: 172) |
| 71 | GCTCAGGTCAGGCAGT | /52MOErG/*/i2MOErC/*/ |
| | GGTT | i2MOErT/*/i2MOErC/*/ |
| | (SEQ ID NO: 10) | i2MOErA/*G*G*T*/iMe-dC/ |
| | | *A*G*G*/iMe-dC/*A*G*/ |
| | | i2MOErT/*/i2MOErG/*/ |
| | | i2MOErG/*/i2MOErT/*/ |
| | | 32MOErT/ |
| | | (SEQ ID NO: 173) |
| 72 | GCTCCTCCCTGCTGGT | /52MOErG/*/i2MOErC/*/ |
| | GATC | i2MOErT/*/i2MOErC/*/ |
| | (SEQ ID NO: 95) | i2MOErC/*T*/iMe-dC/*/ |
| | | iMe-dC/*/iMe-dC/*T*G*/ |
| | | iMe-dC/*T*G*G*/i2MOErT/ |
| | | */i2MOErG/*/i2MOErA/*/ |
| | | i2MOErT/*/32MOErC/ |
| | | (SEQ ID NO: 174) |
| 73 | GCTGGCCCTGGACTCT | 52MOErG/*/i2MOErC/*/ |
| | TCTT | i2MOErT/*/i2MOErG/*/ |
| | (SEQ ID NO: 96) | i2MOErG/*/iMe-dC/*/ |
| | | iMe-dC/*/iMe-dC/ |
| | | *T*G*G*A*/iMe-dC/*T*/ |
| | | iMe-dC/*/i2MOErT/*/ |
| | | i2MOErT/*/i2MOErC/*/ |
| | | i2MOErT/*/32MOErT/ |
| | | (SEQ ID NO: 175) |
| 74 | CCCACCCTCCATCTGT | /52MOErC/*/i2MOErC/*/ |
| | GCAG | i2MOErC/*/i2MOErA/*/ |
| | (SEQ ID NO: 97) | i2MOErC/*/iMe-dC/*/ |
| | | iMe-dC/*T*/iMe-dC/*/ |
| | | iMe-dC/*A*T*/iMe-dC/ |
| | | *T*G*/i2MOErT/*/ |
| | | i2MOErG/*/i2MOErC/*/ |
| | | i2MOErA/*/32MOErG/ |
| | | (SEQ ID NO: 176) |
| 75 | TCCTGCTTTTCCCACT | /52MOErT/*/i2MOErC/*/ |
| | GGCT | i2MOErC/*/i2MOErT/*/ |
| | (SEQ ID NO: 98) | i2MOErG/*/iMe-dC/ |
| | | *T*T*T*T*/iMe-dC/*/ |
| | | iMe-dC/*/iMe-dC/*A*/ |
| | | iMe-dC/*/i2MOErT/*/ |
| | | i2MOErG/*/i2MOErG/*/ |
| | | i2MOErC/*/32MOErT/ |
| | | (SEQ ID NO: 177) |
| 76 | AGCCCTTGAGTGTGGG | /52MOErA/*/i2MOErG/*/ |
| | TCCA | i2MOErC/*/i2MOErC/*/ |
| | (SEQ ID NO: 99) | i2MOErC/ |
| | | *T*T*G*A*G*T*G*T*G*G*/ |
| | | i2MOErG/*/i2MOErT/*/ |
| | | i2MOErC/*/i2MOErC/*/ |
| | | 32MOErA/ |
| | | (SEQ ID NO: 178) |
| 77 | TCCAGCTCTGTGCCTC | /52MOErT/*/i2MOErC/*/ |
| | TAGT | i2MOErC/*/i2MOErA/*/ |
| | (SEQ ID NO: 12) | i2MOErG/*/iMe-dC/*T*/ |
| | | iMe-dC/*T*G*T*G*/ |
| | | iMe-dC/*/iMe-dC/*T*/ |
| | | i2MOErC/*/i2MOErT/*/ |
| | | i2MOErA/*/i2MOErG/*/ |
| | | 32MOErT/ |
| | | (SEQ ID NO: 179) |
| 78 | CTGTCCACCTTCACCC | /52MOErC/*/i2MOErT/*/ |
| | TGCC | i2MOErG/*/i2MOErT/*/ |
| | (SEQ ID NO: 100) | i2MOErC/*/iMe-dC/*A*/ |
| | | iMe-dC/*/iMe-dC/*T*T*/ |
| | | iMe-dC/*A*/iMe-dC/*/ |
| | | iMe-dC/*/i2MOErC/*/ |
| | | i2MOErT/*/i2MOErG/*/ |
| | | i2MOErC/*/32MOErC/ |
| | | (SEQ ID NO: 180) |
| 79 | GCCCTCTGTGGCCTTC | /52MOErG/*/i2MOErC/*/ |
| | CCTG | i2MOErC/*/i2MOErC/*/ |
| | (SEQ ID NO: 101) | i2MOErT/*/iMe-dC/ |
| | | *T*G*T*G*G*/iMe-dC/*/ |
| | | iMe-dC/*T*T*/i2MOErC/*/ |
| | | i2MOErC/*/i2MOErC/*/ |
| | | i2MOErT/*/32MOErG/ |
| | | (SEQ ID NO: 181) |
| 80 | GCCTTAGACCTGCCCT | 52MOErG/*/i2MOErC/*/ |
| | CCTC | i2MOErC/*/i2MOErT/*/ |
| | (SEQ ID NO: 102) | i2MOErT/*A*G*A*/iMe-dC/ |
| | | */iMe-dC/*T*G*/iMe-dC/ |
| | | */iMe-dC/*/iMe-dC/*/ |
| | | i2MOErT/*/i2MOErC/*/ |
| | | i2MOErC/*/i2MOErT/*/ |
| | | 32MOErC/ |
| | | (SEQ ID NO: 182) |

Notes: Unless noted otherwise, oligonucleotides are described from 5′ to 3′. As appreciated by those skilled in the art, internucleotidic linkages connect 5′ and 3′ positions of sugars. Unless otherwise noted (e.g., by “*” which is for a phosphorothioate internucleotidic linkage), an internucleotidic linkage is a natural phosphate linkage. Unless otherwise noted, each of A, T, C and G is independently deoxyadenosine, thymidine, deoxycytidine, and deoxyguanosine, respectively (e.g., as typically found in natural DNA). “2MOEr” indicates a 2′-MOE modification to a sugar: “5” indicates a nucleoside has a 5′-OH group (e.g., when at the 5′-end of an oligonucleotide); “3” indicates a nucleoside has a 3′-OH group (e.g., when at the 3′-end of an oligonucleotide); “i” indicates an nucleoside is in the middle of an oligonucleotide and its 5′- and 3′-positions are bonded to internucleotidic linkages as indicated; “Me-dC” indicates a 5-methyl-2′-deoxycytidine nucleoside. As those skilled in the art appreciates, oligonucleotides may exist in various forms including various salt forms.

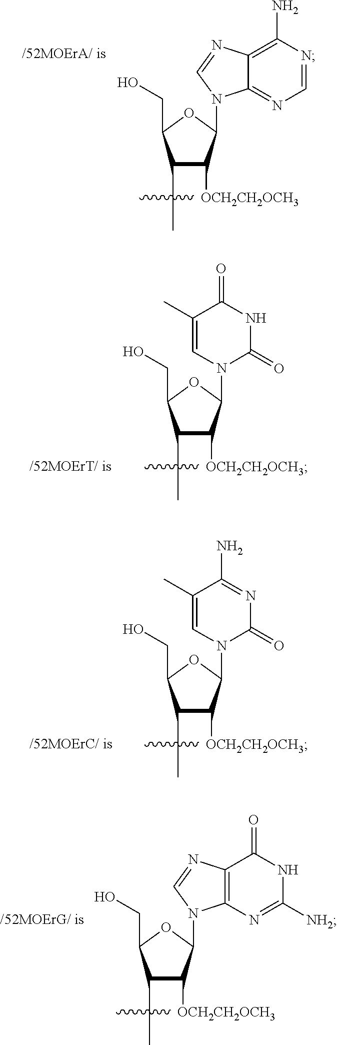

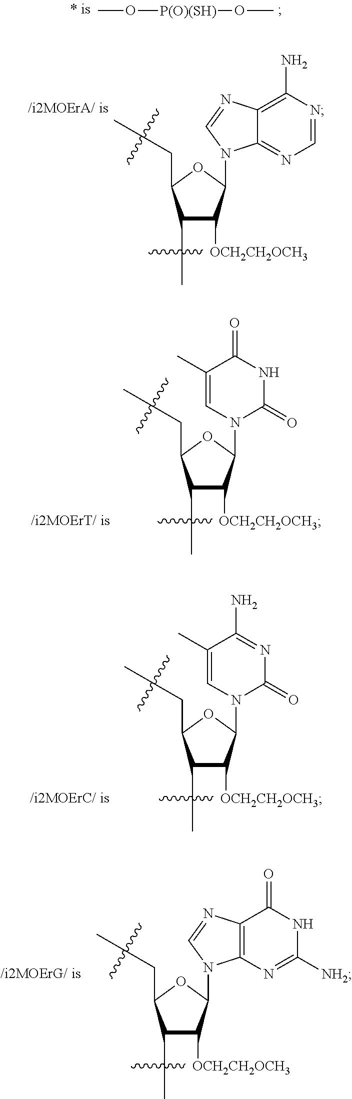

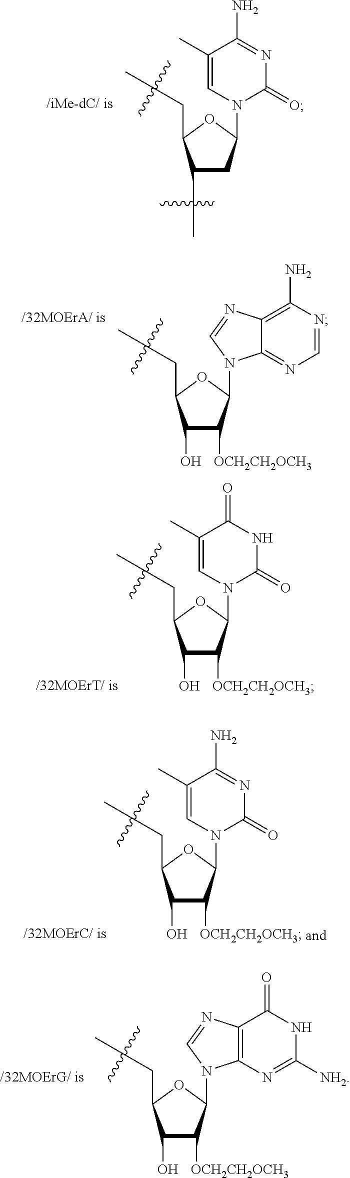

In some embodiments, provided oligonucleotides are capable of hybridizing to a calpain-2 transcript. In some embodiments, provided oligonucleotides can reduce levels of calpain-2 transcripts or products thereof. In some embodiments, provided oligonucleotide can reduce levels of calpain-2 mRNA. In some embodiments, provided oligonucleotide can reduce levels of calpain-2 polypeptides. In some embodiments, provided oligonucleotide can reduce activity levels of calpain-2 polypeptides observed in a system (e.g., a sample, a subject, etc.). In some embodiments, an oligonucleotide is selected from Table 1. In some embodiments, an oligonucleotide is a pharmaceutically acceptable salt of an oligonucleotide selected from Table 1.

In some embodiments, the present disclosure provides oligonucleotides that are particularly effective in reducing levels of calpain-2 transcripts, polypeptides and/or activities.

In some embodiments, an oligonucleotide has the structure of /52MOErA/*/i2MOErT/*/i2MOErC/*/i2MOErA/*/i2MOErG/*T*T*T*/iMe-dC/*T*G*T*A*G*G*/i2MOErC/*/i2MOErT/*/i2MOErT/*/i2MOErC/*/32MOErC/ (SEQ ID NO: 116) or a salt thereof.

In some embodiments, an oligonucleotide has the structure of /52MOErG/*/i2MOErG/*/i2MOErC/*/i2MOErA/*/i2MOErT/*A*/iMe-dC/*T*G*G*T*T*/iMe-dC/*A*G*/i2MOErT/*/i2MOErT/*/i2MOErG/*/i2MOErA/*/32MOErT/ (SEQ ID NO: 141) or a salt thereof.

In some embodiments, an oligonucleotide has the structure of /52MOErG/*/i2MOErC/*/i2MOErT/*/i2MOErC/*/i2MOErA/*G*G*T*/iMe-dC/*A*G*G*/iMe-dC/*A*G*/i2MOErT/*/i2MOErG/*/i2MOErG/*/i2MOErT/*/32MOErT/ (SEQ ID NO: 173) or a salt thereof.

In some embodiments, an oligonucleotide has the structure of /52MOErG/*/i2MOErA/*/i2MOErG/*/i2MOErA/*/i2MOErG/*/iMe-dC/*/iMe-dC/*T*T*T*T*T*G*/iMe-dC/*A*/i2MOErG/*/i2MOErA/*/i2MOErG/*/i2MOErC/*/32MOErT/ (SEQ ID NO: 114) or a salt thereof.

In some embodiments, an oligonucleotide has the structure of /52MOErT/*/i2MOErC/*/i2MOErC/*/i2MOErA/*/i2MOErG/*/iMe-dC/*T*/iMe-dC/*T*G*T*G*/iMe-dC/*/iMe-dC/*T*/i2MOErC/*/i2MOErT/*/i2MOErA/*/i2MOErG/*/32MOErT/ (SEQ ID NO: 179) or a salt thereof.

In some embodiments, the present disclosure provides compositions comprising provided oligonucleotides. In some embodiments, an oligonucleotide composition comprises a provided oligonucleotide or a salt thereof, and various diastereomers and salt thereof. In some embodiments, an oligonucleotide composition comprises a provided oligonucleotide or a salt thereof, and various diastereomers with respect to chiral linkage phosphorus and salt thereof. In some embodiments, oligonucleotides may exist in one or more forms. In some embodiments, oligonucleotides in a composition exist in a salt form. In some embodiments, oligonucleotides in a composition exist in one or more salt forms. In some embodiments, a salt form is a pharmaceutically acceptable salt form. In some embodiments, a salt form is a metal salt. In some embodiments, a salt form is a alkali metal salt. In some embodiments, a salt form is a sodium salt. In some embodiments, a salt form is a potassium salt. In some embodiments, a salt form is a calcium salt. In some embodiments, a salt form is an ammonium salt form (e.g., of N(R′)₃ wherein R′ is as described herein; in some embodiments, each R′ is independently —H or optionally substituted C₁₋₆ alkyl). In some embodiments, an oligonucleotide composition is a liquid composition, wherein an oligonucleotide is dissolved in a solution. In some embodiments, a solution is a buffer. In some embodiments, a solution is a buffered saline. In some embodiments, in a composition acidic internucleotidic linkages, e.g., natural phosphate linkages, phosphorothioate internucleotidic linkages, e.g., independently exist as anionic forms, and the composition comprises one or more types of cations, e.g., Na′, K′, etc.

### Additional Chemical Moieties

In some embodiments, an oligonucleotide comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of oligonucleotides, e.g., stability, half life, activities, delivery, pharmacodynamics properties, pharmacokinetic properties, etc. In some embodiments, certain additional chemical moieties facilitate delivery of oligonucleotides to desired cells, tissues and/or organs, including but not limited the cells of the central nervous system. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides.

Certain useful additional chemical moieties are described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the additional chemical moieties of each of which are incorporated herein by reference.

### Manufacturing

Various technologies are available in the art to manufacture provided oligonucleotide and may be utilized in accordance with the present disclosure. For example, in some embodiments, oligonucleotides are manufactured on solid support using phosphoramidites. In some embodiments, oligonucleotides are manufactured in solution. In some embodiments, manufacturing of oligonucleotides comprise multiple cycles, in each of which one or more nucleoside units, typically one, are added. In some embodiments, a cycle comprises coupling of a phosphoramidite, blocking unreacted 5′-OH groups, modification (e.g., sulfurization), and/or de-blocking protected 5′-OH groups in newly coupled nucleosides. In some embodiments, modification may be performed at the end of cycles when certain lengths of oligonucleotides are achieved.

Certain technologies for manufacturing oligonucleotides are described in U.S. Pat. Nos. 3,687,808, 4,469,863, 4,476,301, 5,177,195, 5,023,243, 5,034,506, 5,166,315, 5,185,444, 5,188,897, 5,214,134, 5,216,141, 5,235,033, 5,264,423, 5,264,564, 5,276,019, 5,278,302, 5,286,717, 5,321,131, 5,399,676, 5,405,938, 5,405,939, 5,434,257, 5,453,496, 5,455,233, 5,466,677, 5,466,677, 5,470,967, 5,476,925, 5,489,677, 5,519,126, 5,536,821, 5,541,307, 5,541,316, 5,550,111, 5,561,225, 5,563,253, 5,571,799, 5,587,361, 5,596,086, 5,602,240, 5,608,046, 5,610,289, 5,618,704, 5,623,070, 5,625,050, 5,633,360, 564,562, 5,663,312, 5,677,437, 5,677,439, 6,160,109, 6,239,265, 6,028,188, 6,124,445, 6,169,170, 6,172,209, 6,277,603, 6,326,199, 6,346,614, 6,444,423, 6,531,590, 6,534,639, 6,608,035, 6,683,167, 6,858,715, 6,867,294, 6,878,805, 7,015,315, 7,041,816, 7,273,933, 7,321,029, U.S. RE39464, U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194. WO 2019/032607, WO 2019/055951, WO 2019/075357, WO 2019/200185, WO 2019/217784, or WO 2019/032612.

In some embodiments, oligonucleotides and/or compositions are provided as stereorandom compositions with respect to chiral linkage phosphorus. For example, when using traditional phosphoramidites comprising N, N-diisopropylamino and 2-cyanoethyloxy groups for oligonucleotide synthesis, chiral linkages may be formed with no or low stereoselectivity. In some embodiments, oligonucleotides are provided as a mixture of various diastereomers and/or salts thereof. In some embodiments, a composition comprises an oligonucleotide, and/or one or more or all of its diastereomers with respect to chiral linkage phosphorus. In some embodiments, an oligonucleotide and/or its diastereomers are independently in one or more forms. In some embodiments, an oligonucleotide and/or its diastereomers are independently in one or more salt forms, e.g., one or more pharmaceutically acceptable salt forms. In some embodiments, for each chiral linkage phosphorus, both the Rp and the Sp configurations are present in the composition. In some embodiments, for each chiral linkage phosphorus, both the Rp and the Sp configurations have a percentage of at least about 10%, 15%, 20%, 25%, 30% 35%, 40%, or 45%. In some embodiments, for a chiral internucleotidic linkage both the Rp and the Sp configurations have a percentage of about 50%. In some embodiments, for each chiral internucleotidic linkage both the Rp and the Sp configurations have a percentage of about 50%. In some embodiments, for each chiral internucleotidic linkage both the Rp and the Sp configurations have a percentage of about 20-80%. In some embodiments, for each chiral internucleotidic linkage both the Rp and the Sp configurations have a percentage of about 30-70%. In some embodiments, for each chiral internucleotidic linkage both the Rp and the Sp configurations have a percentage of about 40-60%. In some embodiments, for each chiral internucleotidic linkage both the Rp and the Sp configurations have a percentage of about 45-55%. In some embodiments, for each linkage phosphorus the Rp configuration independently has a percentage of about 20-80%, 30-70%, 40-60%, or 45-55%, or about 20%, 30%, 40%, 50%, 60%, 70% or 80%.

Amount, concentration, etc., of provided oligonucleotides may be assessed utilizing various technologies in accordance with the present disclosure, e.g., by UV (e.g., at 260 nm), weight, etc. In some embodiments, amount, concentration, etc., of all oligonucleotides present in a composition is assessed. In some embodiments, amount, concentration, etc., include all oligonucleotides share the same constitution (e.g., diastereomers with respect to chiral linkage phosphorus) including all their forms including pharmaceutically acceptable salt forms.

### Calpain-2

In some embodiments, calpain-2 (CAPN2) refers to a gene or a gene product thereof (e.g., a nucleic acid (e.g., DNA, RNA, etc.), a transcript (e.g., a calpain-2 mRNA), a protein encoded thereby (e.g., a calpain-2 polypeptide), etc.) from a species, which may be known as CAPN2, CANP2, CANPL2, mCANP, CANPml, calpain-2, calpain 2, m-calpain, millimolar-calpain, calpain M-type, calpain-2 large subunit, calpain 2 (m/ll) large subunit, calpain large polypeptide L2, calpain-2 catalytic subunit, calcium-activated neural proteinase 2, CANP 2, etc. Various CAPN2 sequences including variants thereof are readily available to those of skill in the art. Various technologies, e.g., assays, cells, animal models, etc., have also been reported and can be utilized for characterization and/or assessment of provided technologies (e.g., oligonucleotides, compositions, methods, etc.) in accordance with the present disclosure.

The CAPN2 gene is reported to encode a calpain-2 protein, which according to various reports comprises 622 or 700 amino acids, depending on isoform, and primarily localizes to cellular cytoplasm and cellular plasma membrane in a variety of tissues including those of a central nervous system (CNS). It has been reported that in some embodiments human calpain-2 comprises multiple domains including, from N-terminal region to C-terminal region: (i) an alpha helix: (ii) a CysPc domain comprising a first protease core domain (PC1) and a second protease core domain (PC2); (iii) a calpain-type beta-sandwich (CBSW) domain; and (iv) a penta-EF-hand in the catalytic large subunit (PEF(L)) domain. Calpain-2 proteins from other species e.g., monkeys, rats, and mice, have been reported to comprise various conserved domains as human calpain-2.

Calpain-2 reportedly belongs to a family of calcium-dependent proteases, and has been reported to cleave a multitude of protein targets including, e.g., actin, cytoplasmic polyadenylation element-binding protein 3 (CPEB3), p35, phosphatase and tensin homolog deleted on chromosome 10 (PTEN), protein tyrosine phosphatase (PTPN13; also known as Fas-associated protein-1 (FAP1)), spectrin, TDP-43, neurofilament-light chain (Wang et al., Expert Opin. Ther. Targets, 2018; Baudry, Cuff. Neuropharmacol., 2019), etc. Activation of calpain-2 has been reported to involve relatively high concentrations of calcium ion (Ca²⁺), with reported ranges comprising near-millimolar amounts, e.g., 400-800 μM. In addition, some studies have suggested that calpain-2 may be capable of being activated by phosphorylation by epidermal growth factor (EGF) or brain-derived neurotrophic factor (BDNF) via extracellular signal-regulated kinase (ERK) (Glading et al., Mol. Cell Biol., 2004; Zadran et al., J. Neurosci., 2010). Calpain-2 has been reported as a regulator of synaptic plasticity and possibly limiting such plasticity (Baudry and Bi, Trends Neurosci., 2017), and has been reported to be involved in excitotoxicity as inhibition of calpain-2 has reduced such toxicity (Wang et al., J. Neurosci., 2013).

Dysregulation of calpain-2 has been reported to be associated with various forms of neurodegeneration. Calpain-2 has been implicated in neurodegenerative conditions, disorders or diseases including amyotrophic lateral sclerosis (ALS), Alzheimer's disease (AD), etc. Another neurodegenerative disease, Wolfram syndrome (WS) has been reported to involve hyperactivation of calpain 2 and calpain-2-associated cell death (Lu et al., Proc. Natl. Acad. Sci., 2014). In addition to progressive neurodegenerative diseases, inhibition of calpain-2 has been reported as a potential target for treating acute neuronal injuries (Wang et al., Expert Opin. Ther. Targets, 2018). Some studies have also implicated calpain-2 in traumatic brain injury (TBI) as calpain-2 deletion or inhibition has been reported to result in amelioration of neuropathological features in a mouse model of TBI (Wang et al., Sci. Adv., 2020). As reported, calpain-2 inhibition may result in improvements in the localization of TDP-43 in TBI: TDP-43 has been reported to form inclusions in some forms of neurodegeneration (Gao et al., J. Neurochem., 2018). As such, calpain-2 may be a potential therapeutic target for both progressive neurodegenerative diseases as well as acute neuronal injury.

### CAPN2-Associated Conditions, Disorders or Diseases

Various conditions, disorders or diseases are reported to be associated with calpain-2 and may be prevented or treated with present disclosure. Generally, a disease, disorder, or condition is associated with calpain-2 if the presence, level, activity, and/or form of calpain-2 and/or products (e.g., transcripts, encoded proteins, etc.) thereof correlates with incidence of and/or susceptibility to the disease, disorder, or condition (e.g., across a relevant population). In some embodiments, a condition, disorder or disease associated with calpain-2 may be treated and/or prevented by reducing expression, level and/or activity of calpain-2 transcripts and/or proteins.

Among other things, the present disclosure provides technologies for preventing and/or treating various conditions, disorders or diseases. In some embodiments, a condition, disorder, or disease is a neurodegenerative disease. In some embodiments, a condition, disorder or disease is amyotrophic lateral sclerosis (ALS). In some embodiments, a condition, disorder or disease is traumatic brain injury (TBI). In some embodiments, a condition, disorder or disease is Alzheimer's disease (AD). In some embodiments, a condition, disorder or disease is Parkinson's disease (PD). In some embodiments, a condition, disorder or disease is frontotemporal dementia (FTD). In some embodiments, a condition, disorder or disease is progressive supranuclear palsy (PSP). In some embodiments, a condition, disorder or disease is corticobasal degeneration (CBD). In some embodiments, a condition, disorder or disease is Wolfram syndrome (WS). In some embodiments, a condition, disorder or disease is Friedreich's ataxia (FRDA). In some embodiments, a condition, disorder or disease is multiple system atrophy (MSA). In some embodiments, a condition, disorder or disease is spinocerebellar ataxia (SCA). In some embodiments, a condition, disorder or disease is spinal muscular atrophy (SMA). In some embodiments, a condition, disorder or disease is Pick's disease (PD). In some embodiments, a condition, disorder or disease is progressive motor atrophy. In some embodiments, a condition, disorder or disease is concussion. In some embodiments, a condition, disorder or disease is spinal cord injury (SCI). In some embodiments, a condition, disorder or disease is chronic traumatic encephalopathy (CTE). In some embodiments, a condition, disorder or disease is seizure. In some embodiments, a condition, disorder or disease is stroke. In some embodiments, a condition, disorder or disease is intracerebral hemorrhage. In some embodiments, a condition, disorder or disease is a tauopathy. In some embodiments, a condition, disorder or disease is associated with Wallerian degeneration. In some embodiments, a condition, disorder or disease is acute glaucoma. In some embodiments, a condition, disorder or disease is cancer. In some embodiments, a condition, disorder or disease is diabetes. In some embodiments, a condition, disorder or disease is chemotherapy-induced peripheral neuropathy.

### Characterization and Assessment

In some embodiments, properties and/or activities of provided oligonucleotides and compositions thereof can be characterized and/or assessed using various technologies available to those skilled in the art, e.g., biochemical assays (e.g., RNase H assays), cell based assays, animal models, clinical trials, etc. Certain useful technologies are described in the Examples. Those skilled in the art reading the present disclosure will readily appreciate that other technologies, e.g., in vitro models (e.g., cell lines) for various conditions, disorders or diseases, animal models for various conditions, disorders or diseases, clinical trials, etc. may be designed and/or utilized to assess provided technologies (e.g., oligonucleotides, compositions, methods, etc.) in accordance with the present disclosure.

### Biological Applications

As appreciated by those skilled in the art, oligonucleotides are useful for many purposes. In some embodiments, provided technologies (e.g., oligonucleotides, compositions, methods, etc.) are useful for reducing levels and/or activities of various calpain-2 transcripts (e.g., RNA) and/or products encoded thereby (e.g., proteins). In some embodiments, provided technologies reduce levels and/or activities calpain-2 RNA transcripts. In some embodiments, provided oligonucleotides and compositions provide knockdown of calpain-2 mRNA. In some embodiments, provided technologies reduce levels of calpain-2 polypeptides. In some embodiments, provided technologies reduce levels of calpain-2 activities, e.g., protease activities.

In some embodiments, the present disclosure provides a method for reducing level of calpain-2 mRNA in a system, comprising administering or delivering to the system an effective amount of an oligonucleotide or an oligonucleotide composition. In some embodiments, the present disclosure provides a method for reducing level of a calpain-2 polypeptide in a system, comprising administering or delivering to the system an effective amount of an oligonucleotide or an oligonucleotide composition. In some embodiments, the present disclosure provides a method for reducing level of a calpain-2 activity in a system, comprising administering or delivering to the system an effective amount of an oligonucleotide or an oligonucleotide composition. In some embodiments, the present disclosure provides a method for reducing level of calpain-2 protease activity in a system, comprising administering or delivering to the system an effective amount of an oligonucleotide or an oligonucleotide composition. In some embodiments, reduction of calpain-2 mRNA and/or polypeptide levels increases mRNA and/or polypeptide levels of a calpain-2 target. In some embodiments, reduction of calpain-2 mRNA and/or polypeptide levels increases mRNA and/or polypeptide levels of calpastatin. In some embodiments, the present disclosure provides technologies for increasing levels of calpastatin mRNA and/or polypeptide in a system, comprising administering or delivering to the system an effective amount of an oligonucleotide or an oligonucleotide composition that targets calpain-2. Calpain-2 has been reported to interact with various partners, e.g., TDP43 and other ALS biomarkers. In some embodiments, the present disclosure provide technologies for modulating interaction between calpain-2 and a partner. In some embodiments, the present disclosure provides technologies for modulating calpain-2 interaction with a partner in a system, comprising administering or delivering to the system an effective amount of an oligonucleotide or an oligonucleotide composition that targets calpain-2.

In some embodiments, a system comprises calpain-2 mRNA. In some embodiments, a system expresses calpain-2 mRNA. In some embodiments, a system expresses calpain-2 polypeptides.

In some embodiments, a system is an in vitro system. In some embodiments, a system is an in vivo system.

In some embodiments, a system comprises a cell. In some embodiments, a system is a cell. In some embodiments, a system comprises a population of cells. In some embodiments, a system is a population of cells. In some embodiments, a cell is a neuronal cell. In some embodiments, a cell is a cell in the neuronal system. In some embodiments, a cell is a cell in CNS. In some embodiments, a cell possesses one or more characteristics, properties and/or activities of a neuronal cell.

In some embodiments, a system is a tissue. In some embodiments, a system comprises a tissue. In some embodiments, a system is an organ. In some embodiments, a system comprises an organ. In some embodiments, a system is a brain or a portion thereof. In some embodiments, a system comprises a brain or a portion thereof. In some embodiments, a system is an organism. In some embodiments, a system comprises an organism. In some embodiments, a system is a subject. In some embodiments, a system is a mammal, e.g., a mouse, rat, monkey, etc. In some embodiments, a system is a human.

In some embodiments, a level is reduced by at least about 10%, 15%, 20%, 25%, 30%, 35%, 40%, 45%, 50%, 60%, 70%, 75%, 80%, 85%, 90%, or 95% compared to absence of a provided oligonucleotide or composition and/or presence of a reference oligonucleotide or composition. In some embodiments, such a reduction is achieved at certain oligonucleotide concentrations (e.g., 1 nM, 5 nM, 10 nM, 100 nM, 500 nM, 1 uM, 5 uM, etc.) or doses. In some embodiments, such a reduction is achieved is a system, e.g., various suitable assay (e.g., in vitro cell-based assays, assays described in the Examples, etc.). In some embodiments, a reference composition comprises no oligonucleotides targeting calpain-2. In some embodiments, a reference oligonucleotide targets a different nucleic acid than calpain-2. In some embodiments, a level is of mRNA, e.g., calpain 2 mRNA. In some embodiments, a level is of a polypeptide, e.g., calpain 2 protein. In some embodiments, a level is reduced by at least about 10%. In some embodiments, a level is reduced by at least about 20%. In some embodiments, a level is reduced by at least about 30%. In some embodiments, a level is reduced by at least about 40%. In some embodiments, a level is reduced by at least about 50%. In some embodiments, a level is reduced by at least about 60%. In some embodiments, a level is reduced by at least about 70%. In some embodiments, a level is reduced by at least about 75%. In some embodiments, a level is reduced by at least about 80%. In some embodiments, a level is reduced by at least about 85%. In some embodiments, a level is reduced by at least about 90%. In some embodiments, a level is reduced by at least about 95%. In some embodiments, level of calpain-2 mRNA is reduced about or at least about 40%, 45%, 50%, 55%, 60%, 65%, 70%, 75%, 80%, 85%, 90% or 95% at about 20 uM oligonucleotides when assessed, e.g., using an assay in the Examples; in some embodiments, it is reduced about or at least about 30%, 35%, 40%, 45%, 50%, 55%, 60%, 65%, 70%, 75%, 80%, 85% or 90% at about 15 uM oligonucleotides; in some embodiments, it is reduced about or at least about 30%, 35%, 40%, 45%, 50%, 55%, 60%, 65%, 70%, 75% or 80% at about 10 uM oligonucleotides; in some embodiments, it is reduced about or at least about 20%, 25%, 30%, 35%, 40%, 45% 50%, 55%, 60%, 65% or 70% at about 5 uM oligonucleotides; in some embodiments, it is reduced about or at least about 20%, 25%, 30%, 35%, 40%, 45%, 50%, 55%, or 60% 2.5 uM oligonucleotides; in some embodiments, it is reduced about or at least about 20%, 25%, 30%, 35%, 40%, 45%, 50%, 55%, or 60% at about 1.25 uM oligonucleotides; in some embodiments, it is reduced about or at least about 10%, 15%, 20%, 25%, 30%, 35% or 40% at about 0.63 uM oligonucleotides; and in some embodiments, it is reduced about or at least about 10%, 15%, 20%, 25% or 30% at about 0.32 uM oligonucleotides. In some embodiments, a reduction is about or at least about 50%. In some embodiments, a reduction is about or at least about 55%. In some embodiments, a reduction is about or at least about 60%. In some embodiments, a reduction is about or at least about 65%. In some embodiments, a reduction is about or at least about 70%. In some embodiments, a reduction is about or at least about 75%. In some embodiments, a reduction is about or at least about 80%. In some embodiments, a reduction is about or at least about 85%. In some embodiments, a reduction is about or at least about 90%. In some embodiments, a reduction is about or at least about 95%. In some embodiments, a reduction is assessed at or after about 1, 2, 3, 4, 5, 6, 7, 8, 9, 10, 11, 12, 13, 14, 15, 16, 17, 18, 19, 20, 21, 22, 23, 24 or 25 days, or about 1, 2, 3 or 4 weeks following administering or delivering a provided oligonucleotide or composition. In some embodiments, a reduction is assessed at or after about 1, 2, 3, 4, 5, 6, 7, 8, 9, 10, 11, 12, 13, 14, 15, 16, 17, 18, 19, 20, 21, 22, 23, 24 or 25 days, or about 1, 2, 3 or 4 weeks following removal or washout of a provided oligonucleotide or composition. In some embodiments, a reduction is assessed at day 0. In some embodiments, a reduction is assessed at about day 3. In some embodiments, a reduction is assessed at about day 10. In some embodiments, a reduction is assessed at about day 14. In some embodiments, a reduction is assessed at about day 21. In some embodiments, reductions of at one or more assessments, e.g., of calpain mRNA levels, are independently about or at least about 50%, 55%, 60%, 65%, 70%, 75%, 80%, 85%, 90%, 95%, 96%, 97%, 98% or 99%; in some embodiments, reductions are independently about or at least about 75%; in some embodiments, reductions are about or at least about 80%. In some embodiments, reductions of one or more assessments, e.g., of calpain 2 protein or activity, are independently about or at least about 20%, 25%, 30%, 35%, 40%, 45%, 50%, 55%, 60%, 65%, 70%, 75% or 80%: in some embodiments, reductions are about or at least about 20%; in some embodiments, reductions are about or at least about 25%; in some embodiments, reductions are about or at least about 30%; in some embodiments, reductions are about or at least about 35%; in some embodiments, reductions are about or at least about 40%; in some embodiments, reductions are about or at least about 45%; in some embodiments, reductions are about or at least about 50%; in some embodiments, reductions are about or at least about 55%; in some embodiments, reductions are about or at least about 60%; in some embodiments, reductions are about or at least about 70%. In some embodiments, reductions are relative to absence of oligonucleotides. In some embodiments, reductions are relative to presence of reference oligonucleotides or compositions, e.g., oligonucleotides targeting TUG1 as described herein. In some embodiments, assessments are performed according to those described in the Examples, e.g., in some embodiments, using iPSC-derived motor neurons with oligonucleotide concentrations at about 20 uM with gymnotic delivery.

In some embodiments, activity of a provided oligonucleotide or oligonucleotide composition may be assessed by IC50 which is the concentration to reduce a level, e.g., of calpain-2 mRNA, polypeptide, activity, etc., by 50% in a suitable condition, e.g., cell-based in vitro assays, assays described in the Examples, etc. In some embodiments, provided oligonucleotides or compositions have an IC50 of about or no more than about 0.001, 0.01, 0.1, 0.5, 1, 2, 5, 10, 50, 100, 200, 500, 1000, 2000, 5000 or 10000 nM. In some embodiments, an oligonucleotide has an IC50 of about or no more than about 10000 nM. In some embodiments, an oligonucleotide has an IC50 of about or no more than about 5000 nM. In some embodiments, an oligonucleotide has an IC50 of about or no more than about 2000 nM. In some embodiments, an oligonucleotide has an IC50 of about or no more than about 1000 nM. In some embodiments, an IC50 is about or no more than about 500 nM. In some embodiments, an IC50 is about or no more than about 200 nM. In some embodiments, an IC50 is about or no more than about 100 nM. In some embodiments, an IC50 is about or no more than about 50 nM. In some embodiments, an IC50 is about or no more than about 20 nM. In some embodiments, an IC50 is about or no more than about 10 nM. In some embodiments, an IC50 is about or no more than about 5 nM. In some embodiments, an IC50 is about or no more than about 2 nM. In some embodiments, an IC50 is about or no more than about 1 nM.

In some embodiments, provided oligonucleotides and compositions are useful for treating various conditions, disorders or diseases, by reducing levels and/or activities of calpain-2 transcripts and/or products encoded thereby that are associated with the conditions, disorders or diseases.

In some embodiments, the present disclosure provides method for preventing a condition, disorder or disease, comprising administering or delivering to a subject susceptible thereto an effective amount of an oligonucleotide or composition of the present disclosure. In some embodiments, the present disclosure provides method for treating a condition, disorder or disease, comprising administering or delivering to a subject suffering therefrom an effective amount of an oligonucleotide or composition of the present disclosure.

Various conditions, disorders or diseases associated with calpain-2 may be prevented or treated with provided technologies. In some embodiments, a subject benefits from reduction of levels of calpain-2 transcripts, polypeptides and/or activities in certain cells, tissues and/or organs.

In some embodiments, a condition, disorder or disease is a neurodegenerative condition, disorder or disease. In some embodiments, a condition, disorder or disease is or comprises Wallerian degeneration. In some embodiments, Wallerian degeneration is associated with damage. In some embodiments, Wallerian degeneration is associated with stress. In some embodiments, a condition, disorder or disease is associated with Wallerian degeneration. In some embodiments, a condition, disorder or disease is amyotrophic lateral sclerosis (ALS). In some embodiments, a condition, disorder or disease is neuropathy. In some embodiments, a condition, disorder or disease is peripheral neuropathy. In some embodiments, a condition, disorder or disease is peripheral neuropathy induced by chemotherapy. In some embodiments, a condition, disorder or disease is Parkinson's disease. In some embodiments, a condition, disorder or disease is Huntington's disease. In some embodiments, a condition, disorder or disease is Alzheimer's disease. In some embodiments, a condition, disorder or disease is frontotemporal dementia. In some embodiments, a condition, disorder or disease is brain injury. In some embodiments, a condition, disorder or disease is traumatic brain injury. In some embodiments, a condition, disorder or disease is progressive supranuclear palsy. In some embodiments, a condition, disorder or disease is corticobasal degeneration. In some embodiments, a condition, disorder or disease is Wolfram Syndrome. In some embodiments, a condition, disorder or disease is Friedreich's Ataxia. In some embodiments, a condition, disorder or disease is Multiple System Atrophy. In some embodiments, a condition, disorder or disease is Spinal Cerebellar Ataxia. In some embodiments, a condition, disorder or disease is Spinal Muscular Atrophy (SMA). In some embodiments, a condition, disorder or disease is Pick's Disease. In some embodiments, a condition, disorder or disease is progressive motor atrophy. In some embodiments, a condition, disorder or disease is associated with neuronal damage. In some embodiments, a condition, disorder or disease is associated with neuronal cell damage. In some embodiments, a condition, disorder or disease is associated with neuronal cell death. In some embodiments, a condition, disorder or disease is axonal degeneration. In some embodiments, a condition, disorder or disease is axonal degeneration is ALS.

Various technologies may be utilized to administer or deliver provided oligonucleotides or compositions. In some embodiments, an oligonucleotide or a composition is administered or delivered orally. In some embodiments, an oligonucleotide or a composition is administered or delivered via parenteral routes. In some embodiments, parenteral routes include intravenous, intrarterial, intramuscular, intradermal, subcutaneous, intranasal, and intraperitoneal routes. In some embodiments, an oligonucleotide or a composition is administered or delivered via intraocular, intraorbital, subconjuctival, intravitreal, subretinal, transscleral or introcochlear route. In some embodiments, an oligonucleotide or a composition is administered or delivered parenterally. In some embodiments, an oligonucleotide or a composition is administered or delivered intrathecally. In some embodiments, an oligonucleotide or a composition is administered or delivered intravenously. In some embodiments, oligonucleotides are administered or delivered as a liquid composition. In some embodiments, oligonucleotides are dissolved in a liquid, e.g., a buffered saline such as aCSF, for administration or delivery.

In some embodiments, an oligonucleotide or composition may be utilized in combination with another therapy, e.g., another therapeutic agent.

In some embodiments, provided technologies, e.g., oligonucleotides, compositions, methods, etc., delay or prevent onset of or more symptoms and/or hallmarks of a condition, disorder or disease. In some embodiments, provided technologies delay, slow down, or prevent progression of a condition, disorder or disease. In some embodiments, provided technologies alleviate, ameliorate, relieve, inhibit, prevent, delay onset of, reduce severity of, and/or reduce incidence of one or more symptoms or features of a disease, disorder, and/or condition. In some embodiments, provided technologies improve performance of a subject in one or more assessments. In some embodiments, provided technologies improve performance of a subject in one or more clinical assessments. In some embodiments, provided technologies independently improve one or more clinical assessment results of a subject.

### Pharmaceutical Compositions

In some embodiments, the present disclosure provides pharmaceutical compositions comprising a provided compound, e.g., an oligonucleotide, or a pharmaceutically acceptable salt thereof, and a pharmaceutical carrier. In some embodiments, for example, for therapeutic and clinical purposes, oligonucleotides of the present disclosure are provided as pharmaceutical compositions.

As appreciated by those skilled in the art, oligonucleotides can be provided in various forms. In some embodiments, oligonucleotides can be in acid forms, e.g., for natural phosphate linkages, in the form of —OP(O)(OH)O—; for phosphorothioate internucleotidic linkages, in the form of —OP(O)(SH)O—; etc. In some embodiments, provided oligonucleotides can be in salt forms, e.g., for natural phosphate linkages, in the form of —OP(O)(ONa)O— in sodium salts; for phosphorothioate internucleotidic linkages, in the form of —OP(O)(SNa)O— in sodium salts, etc. Unless otherwise noted, oligonucleotides of the present disclosure can exist in acid, base and/or salt forms. In some embodiments, a composition comprises one or more forms of an oligonucleotide. In some embodiments, a composition comprises one or more salt forms of an oligonucleotide. In some embodiments, a composition comprises one or more pharmaceutically acceptable salt forms of an oligonucleotide.

When used as therapeutics, a provided oligonucleotide or composition is typically administered as a pharmaceutical composition. In some embodiments, a pharmaceutical composition is suitable for administration or delivery of an oligonucleotide to an area or portion of a body affected by a condition, disorder or disease. In some embodiments, a pharmaceutical composition comprises a therapeutically effective amount of a provided oligonucleotide or a pharmaceutically acceptable salt thereof, and a pharmaceutically acceptable carrier. In some embodiments, a pharmaceutical composition comprises a therapeutically effective amount of oligonucleotides which are diastereomers of each other, wherein the oligonucleotides exist in one or more forms. In some embodiments, a pharmaceutical composition comprises a therapeutically effective amount of oligonucleotides which are diastereomers of each other with respect to chiral linkage phosphorus, wherein the oligonucleotides exist in one or more forms.

In some embodiments, a pharmaceutically acceptable carrier is a buffer. In some embodiments, a pharmaceutically acceptable carrier is a buffered saline. In some embodiments, a pharmaceutically acceptable carrier is artificial cerebrospinal fluid. In some embodiments, a composition is a liquid composition comprising dissolved oligonucleotides.

In some embodiments, a pharmaceutical composition is formulated for intravenous injection, oral administration, buccal administration, inhalation, nasal administration, topical administration, ophthalmic administration or otic administration. In some embodiments, a pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop. In some embodiments, a pharmaceutical composition is formulated for intrathecal administration.

As those skilled in the art will appreciate, oligonucleotides may exist in various salt forms. In some embodiments, a salt is a pharmaceutically acceptable salt. In some embodiments, a pharmaceutical composition comprises an oligonucleotide, optionally in its salt form, and a sodium salt. In some embodiments, a pharmaceutical composition comprises an oligonucleotide, optionally in its salt form, and sodium chloride. In some embodiments, each hydrogen ion of an oligonucleotide that may be donated to a base (e.g., under conditions of an aqueous solution, a pharmaceutical composition, etc.) is replaced by a non-H⁺ cation. For example, in some embodiments, a pharmaceutically acceptable salt of an oligonucleotide is an all-metal ion salt, wherein each hydrogen ion (for example, of —OH, —SH, etc.) of each internucleotidic linkage (e.g., a natural phosphate linkage, a phosphorothioate internucleotidic linkage, etc.) is replaced by a metal ion. Various suitable metal salts for pharmaceutical compositions are widely known in the art and can be utilized in accordance with the present disclosure. In some embodiments, a pharmaceutically acceptable salt is a sodium salt. In some embodiments, a pharmaceutically acceptable salt is magnesium salt. In some embodiments, a pharmaceutically acceptable salt is a calcium salt. In some embodiments, a pharmaceutically acceptable salt is a potassium salt. In some embodiments, a pharmaceutically acceptable salt is an ammonium salt (cation N(R′)₄⁺). In some embodiments, a pharmaceutically acceptable salt comprises one and no more than one types of cation. In some embodiments, a pharmaceutically acceptable salt comprises two or more types of cation. In some embodiments, a cation is Li⁺, Na⁺, K⁺, Mg²⁺ or Ca²⁺. In some embodiments, a pharmaceutically acceptable salt is an all-sodium salt. In some embodiments, a pharmaceutically acceptable salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate internucleotidic linkage (acid form —O—P(O)(SH)—O—), if any, exists as its sodium salt form (—O—P(O)(SNa)—O—).

In some embodiments, an oligonucleotide or a composition, e.g., a pharmaceutical composition, is provided as solid. In some embodiments, an oligonucleotide or a composition, e.g., a pharmaceutical composition, is lyophilized.

In some embodiments, an oligonucleotide or a composition, e.g., a pharmaceutical composition, is stored at a temperature lower than an ambient temperature, e.g., at about or no more than about −78, −20, 0, 4, or 10° C.

Various technologies for delivering nucleic acids and/or oligonucleotides are known in the art and can be utilized in accordance with the present disclosure. For example, a variety of supramolecular nanocarriers can be used to deliver nucleic acids. Example nanocarriers include liposomes, cationic polymer complexes and various polymeric compounds. Complexation of nucleic acids with various polycations is another approach for intracellular delivery; this includes use of PEGylated polycations, polyethyleneamine (PEI) complexes, cationic block co-polymers, and dendrimers. Several cationic nanocarriers, including PEI and polyamidoamine dendrimers may help to release contents from endosomes. Other approaches may include use of polymeric nanoparticles, microspheres, liposomes, dendrimers, biodegradable polymers, conjugates, prodrugs, inorganic colloids such as sulfur or iron, antibodies, implants, biodegradable implants, biodegradable microspheres, osmotically controlled implants, lipid nanoparticles, emulsions, oily solutions, aqueous solutions, biodegradable polymers, poly(lactide-coglycolic acid), poly(lactic acid), liquid depot, polymer micelles, quantum dots and lipoplexes. In some embodiments, an oligonucleotide is conjugated to another molecule.

In some embodiments, oligonucleotides are administered or delivered via gymnotic uptake.

In some embodiments, oligonucleotides or compositions are formulated for a variety of modes of administration, including systemic and topical or localized administration. Techniques and formulations generally may be found in Remington, The Science and Practice of Pharmacy (20th ed. 2000).

In certain embodiments, oligonucleotides or compositions are delivered to CNS. In certain embodiments, oligonucleotides and compositions are delivered to cerebrospinal fluid. In certain embodiments, oligonucleotides and compositions are administered to brain parenchyma. In certain embodiments, oligonucleotides and compositions are delivered to an animal/subject by intrathecal administration, or intracerebroventricular administration. Broad distribution of oligonucleotides and compositions may be achieved with methods of administration described herein and/or known in the art.

In certain embodiments, parenteral administration is by injection, by, e.g., a syringe, a pump, etc. In certain embodiments, an injection is a bolus injection. In certain embodiments, an injection is administered directly to a tissue or location, such as cerebrospinal fluid, striatum, caudate, cortex, hippocampus and/or cerebellum.

Provided oligonucleotides and compositions thereof are effective over a wide dose range. In some embodiments, a dose is from about 0.01 to about 1000 mg, from about 0.5 to about 100 mg, from about 1 to about 50 mg, or from about 5 to about 100 mg. Exact doses may depend upon routes of administration, forms in which oligonucleotides are administered, subjects (e.g., body weight, age, body surface area, etc.), conditions, disorders or diseases, and/or preferences and experiences of physicians. In some embodiments, a fixed dose is administered. In some embodiments, a provided oligonucleotide or composition is administered or delivered, e.g., by injection or infusion, once every week, every two weeks, every month, every two months, every 90 days, every 3 months, every 6 months, every 9 months, or once a year. In some embodiments, two or more doses are about the same amount. In some embodiments, one or more doses are independently more than one or more other doses. For example, in some embodiments, one or more loading doses each independently of a higher amount are administered before one or more maintenance doses each independently of a lower amount. In some embodiments, two or more or all loading doses are about the same amount. In some embodiments, a loading dose is of a higher amount than another loading dose. In some embodiments, two or more or all maintenance doses are about the same amount. In some embodiments, a maintenance dose is of a higher amount than another maintenance dose.

### Example Embodiments

Among other things, the present disclosure provides the following Example Embodiments:
1. An oligonucleotide, wherein:
   the base sequence of the oligonucleotide comprises 10 or more contiguous nucleobases of ATCAGTTTCTGTAGGCTTCC (SEQ ID NO: 1), wherein each T is optionally and independently replaced with U; and
   the oligonucleotide comprises a modified nucleobase, a modified sugar or a modified internucleotidic linkage.
2. The oligonucleotide of Embodiment 1, wherein the base sequence of the oligonucleotide is ATCAGTTTCTGTAGGCTTCC (SEQ ID NO: 8).
3. An oligonucleotide, wherein:
   the base sequence of the oligonucleotide comprises 10 or more contiguous nucleobases of GGCATACTGGTTCAGTTGAT (SEQ ID NO: 2), wherein each T is optionally and independently replaced with U; and
   the oligonucleotide comprises a modified nucleobase, a modified sugar or a modified internucleotidic linkage.
4. The oligonucleotide of Embodiment 3, wherein the base sequence of the oligonucleotide is

(SEQ ID NO: 9)
GGCATACTGGTTCAGTTGAT.

5. An oligonucleotide, wherein:
   the base sequence of the oligonucleotide comprises 10 or more contiguous nucleobases of GCTCAGGTCAGGCAGTGGTT (SEQ ID NO: 3), wherein each T is optionally and independently replaced with U; and
   the oligonucleotide comprises a modified nucleobase, a modified sugar or a modified internucleotidic linkage.
6. The oligonucleotide of Embodiment 5, wherein the base sequence of the oligonucleotide is

(SEQ ID NO: 10)
GCTCAGGTCAGGCAGTGGTT.

7. An oligonucleotide, wherein:
   the base sequence of the oligonucleotide comprises 10 or more contiguous nucleobases of GAGAGCCTTTTTGCAGAGCT (SEQ ID NO: 4), wherein each T is optionally and independently replaced with U; and
   the oligonucleotide comprises a modified nucleobase, a modified sugar or a modified internucleotidic linkage.
8. The oligonucleotide of Embodiment 7, wherein the base sequence of the oligonucleotide is

(SEQ ID NO: 11)
GAGAGCCTTTTTGCAGAGCT.

9. An oligonucleotide, wherein:
   the base sequence of the oligonucleotide comprises 10 or more contiguous nucleobases of TCCAGCTCTGTGCCTCTAGT (SEQ ID NO: 5), wherein each T is optionally and independently replaced with U; and
   the oligonucleotide comprises a modified nucleobase, a modified sugar or a modified internucleotidic linkage.
10. The oligonucleotide of Embodiment 9, wherein the base sequence of the oligonucleotide is

(SEQ ID NO: 12)
TCCAGCTCTGTGCCTCTAGT.

11. An oligonucleotide, wherein:
   the base sequence of the oligonucleotide comprises 10 or more contiguous nucleobases of GTTCCAGCTTGGGCAGTTGT (SEQ ID NO: 6), wherein each T is optionally and independently replaced with U; and
   the oligonucleotide comprises a modified nucleobase, a modified sugar or a modified internucleotidic linkage.
12. The oligonucleotide of Embodiment 11, wherein the base sequence of the oligonucleotide is

(SEQ ID NO: 13)
GTTCCAGCTTGGGCAGTTGT.

13. An oligonucleotide, wherein:
   the base sequence of the oligonucleotide comprises 10 or more contiguous nucleobases of GGAAGCTTAGTCCTTGGCTG (SEQ ID NO: 7), wherein each T is optionally and independently replaced with U; and
   the oligonucleotide comprises a modified nucleobase, a modified sugar or a modified internucleotidic linkage.
14. The oligonucleotide of Embodiment 13, wherein the base sequence of the oligonucleotide is

(SEQ ID NO: 14)
GGAAGCTTAGTCCTTGGCTG.

15. The oligonucleotide of any one of the preceding Embodiments, wherein the oligonucleotide comprises a 5′-wing-gap-wing-3′ structure.
16. The oligonucleotide of any one of the preceding Embodiments, wherein there are about 3-10 nucleosides in the 5′-wing.
17. The oligonucleotide of any one of the preceding Embodiments, wherein there are 5 nucleosides in the 5′-wing.
18. The oligonucleotide of any one of the preceding Embodiments, wherein each sugar in the 5′-wing is independently a modified sugar.
19. The oligonucleotide of any one of the preceding Embodiments, wherein a sugar in a 5′-wing is a 2′-OR modified sugar wherein R^{s} is C₁₋₆ aliphatic.
20. The oligonucleotide of any one of the preceding Embodiments, wherein a sugar in a 5′-wing is a 2′-MOE modified sugar.
21. The oligonucleotide of any one of the preceding Embodiments, wherein a sugar in a 5′-wing is a 2′-OMe modified sugar.
22. The oligonucleotide of any one of the preceding Embodiments, wherein a sugar in a 5′-wing is a bicyclic sugar.
23. The oligonucleotide of Embodiment 22, wherein the bicyclic sugar is a LNA sugar.
24. The oligonucleotide of Embodiment 22, wherein the bicyclic sugar is a cEt sugar.
25. The oligonucleotide of any one of Embodiments 1-19, wherein each sugar in a 5′-wing is independently a 2′-OR^{s} modified sugar wherein R^{s} is C₁₋₆ aliphatic.
26. The oligonucleotide of any one of Embodiments 1-19, wherein each sugar in a 5′-wing is independently a 2′-MOE modified sugar.
27. The oligonucleotide of any one of the preceding Embodiments, wherein there are about 8-15 nucleosides in the gap.
28. The oligonucleotide of any one of the preceding Embodiments, wherein there are 10 nucleosides in the gap.
29. The oligonucleotide of any one of the preceding Embodiments, wherein each sugar in the gap is independently a natural DNA sugar.
30. The oligonucleotide of any one of the preceding Embodiments, wherein the gap contains no cytosine.
31. The oligonucleotide of any one of the preceding Embodiments, wherein the gap comprises one or more 5-methylcytosine.
32. The oligonucleotide of any one of the preceding Embodiments, wherein there are about 3-10 nucleosides in the 3′-wing.
33. The oligonucleotide of any one of the preceding Embodiments, wherein there are 5 nucleosides in the 3′-wing.
34. The oligonucleotide of any one of the preceding Embodiments, wherein each sugar in the 3′-wing is independently a modified sugar.
35. The oligonucleotide of any one of the preceding Embodiments, wherein a sugar in a 3′-wing is a 2′-OR^{s} modified sugar wherein R^{s} is C₁₋₆ aliphatic.
36. The oligonucleotide of any one of the preceding Embodiments, wherein a sugar in a 3′-wing is a 2′-MOE modified sugar.
37. The oligonucleotide of any one of the preceding Embodiments, wherein a sugar in a 3′-wing is a 2′-OMe modified sugar.
38. The oligonucleotide of any one of the preceding Embodiments, wherein a sugar in a 3′-wing is a bicyclic sugar.
39. The oligonucleotide of Embodiment 38, wherein the bicyclic sugar is a LNA sugar.
40. The oligonucleotide of Embodiment 38, wherein the bicyclic sugar is a cEt sugar.
41. The oligonucleotide of any one of Embodiments 1-35, wherein each sugar in a 3′-wing is independently a 2′-OR^{s} modified sugar wherein R^{s} is C₁₋₆ aliphatic.
42. The oligonucleotide of any one of Embodiments 1-35, wherein each sugar in a 3′-wing is independently a 2′-MOE modified sugar.
43. The oligonucleotide of any one of the preceding Embodiments, wherein the oligonucleotide comprises a modified internucleotidic linkage.
44. The oligonucleotide of Embodiment 43, wherein the modified internucleotidic linkage is a phosphorothioate internucleotidic linkage.
45. The oligonucleotide of any one of the preceding Embodiments, wherein each internucleotidic linkage is independently a modified internucleotidic linkage.
46. The oligonucleotide of any one of the preceding Embodiments, wherein each internucleotidic linkage is independently a phosphorothioate internucleotidic linkage.
47. An oligonucleotide having the structure of:
   /52MOErA/*/i2MOErT/*/i2MOErC/*/i2MOErA/*/i2MOErG/*T*T*T*/iMe-dC/*T*G*T*A*G*G*/i2MOErC/*/i2MOErT/*/i2MOErT/*/i2MOErC/*/32MOErC/ (SEQ ID NO: 116) or a salt thereof, wherein:

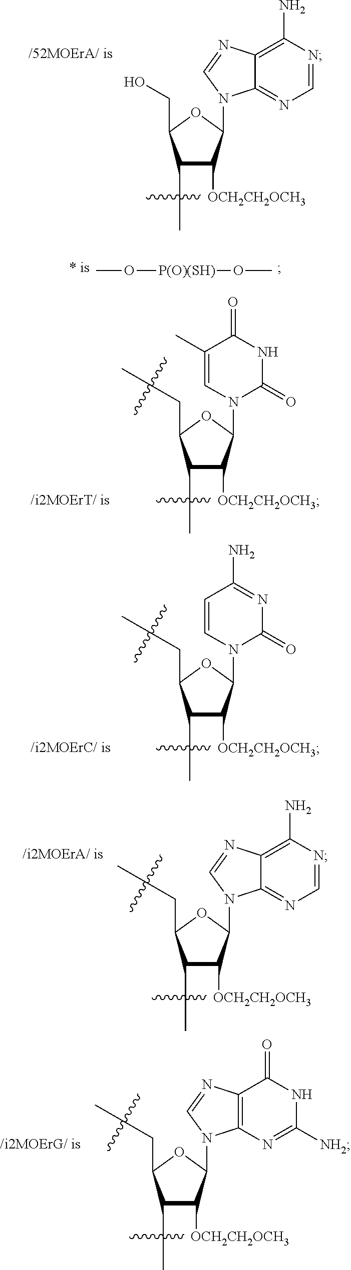

each of A, T and G is independently deoxyadenosine, thymidine, and deoxyguanosine,

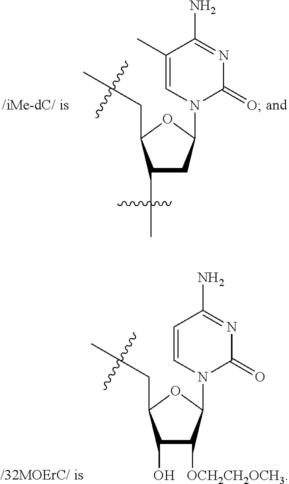

48. An oligonucleotide having the structure of:
   /52MOErG/*/i2MOErG/*/i2MOErC/*/i2MOErA/*/i2MOErT/*A*/iMe-dC/*T*G*G*T*T*/iMe-dC/*A*G*/i2MOErT/*/i2MOErT/*/i2MOErG/*/i2MOErA/*/32MOErT/ (SEQ ID NO. 1411 or a salt thereof, wherein:

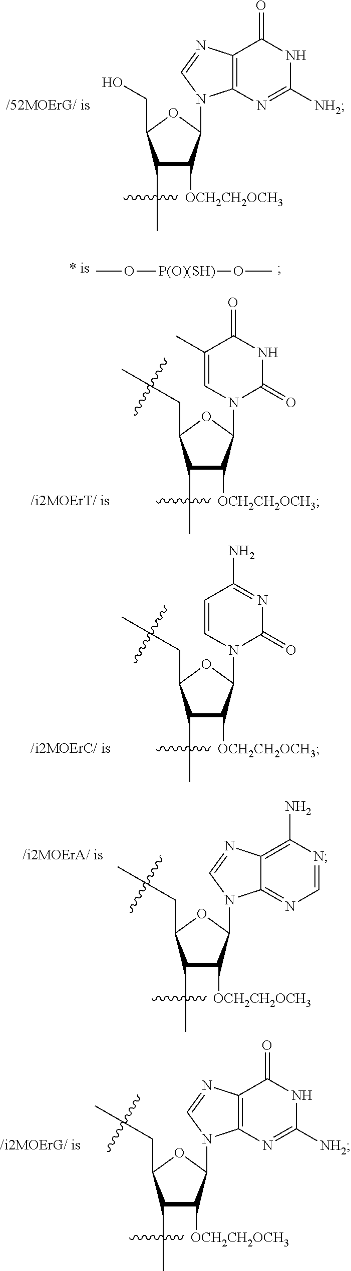

each of A, T and G is independently deoxyadenosine, thymidine, and deoxyguanosine, respectively:

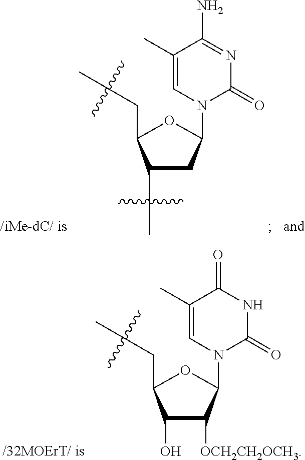

49. An oligonucleotide having the structure of:
   /52MOErG/*/i2MOErC/*/i2MOErT/*/i2MOErC/*/i2MOErA/*G*G*T*/iMe-dC/*A*G*G*/iMe-dC/*A*G*/i2MOErT/*/i2MOErG/*/i2MOErG/*/i2MOErT/*/32MOErT/ (SEQ ID NO: 173) or a salt thereof, wherein:

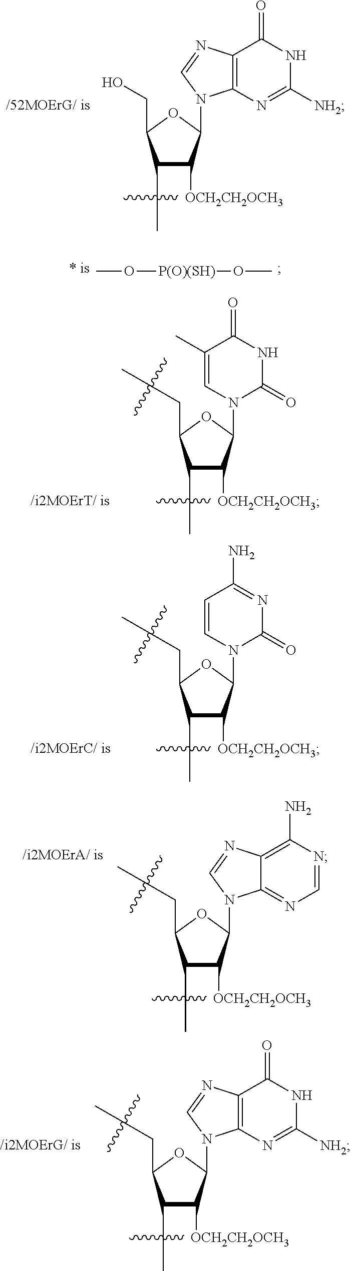

each of A, T and G is independently deoxyadenosine, thymidine, and deoxyguanosine, respectively;

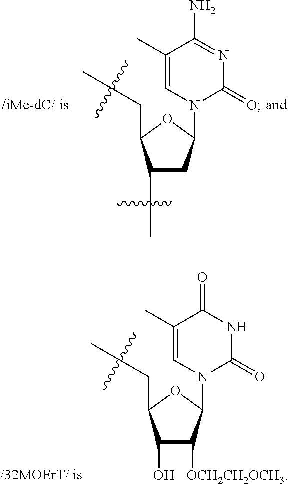

50. An oligonucleotide having the structure of
   /52MOErG/*/i2MOErA/*/i2MOErG/*/i2MOErA/*/i2MOErG/*/iMe-dC/*/iMe-dC/*T*T*T*T*T*G*/iMe-dC/*A*/i2MOErG/*/i2MOErA/*/i2MOErG/*/i2MOErC/*/32MOErT/ (SEQ ID NO: 114) or a salt thereof, wherein:

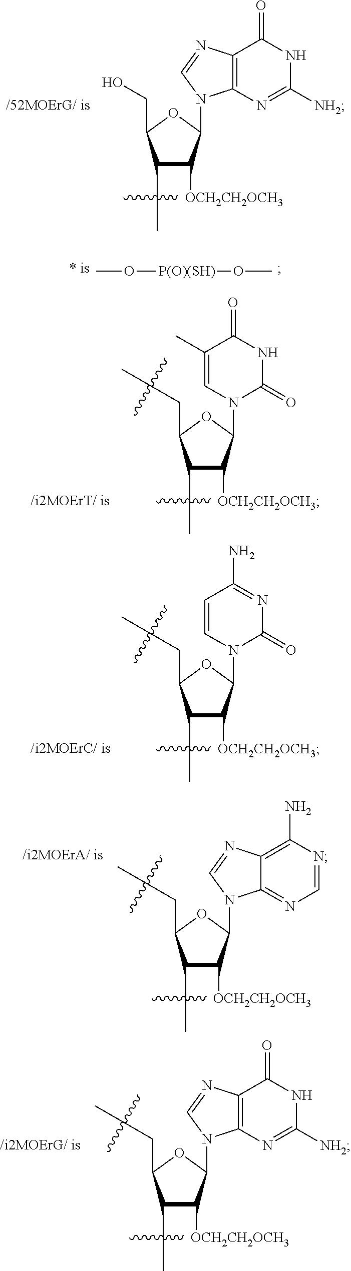

each of A, T and G is independently deoxyadenosine, thymidine, and deoxyguanosine, respectively:

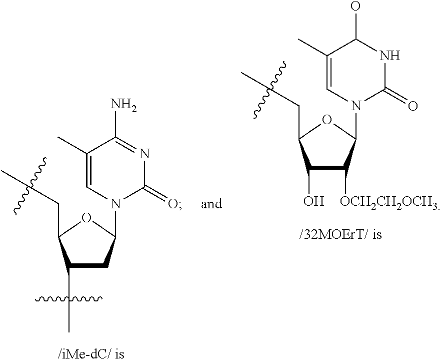

51. An oligonucleotide having the structure of:
   /52MOErT/*/i2MOErC/*/i2MOErC/*/i2MOErA/*/i2MOErG/*/iMe-dC/*T*/iMe-dC/*T*G*T*G*/iMe-dC/*/iMe-dC/*T*/i2MOErC/*/i2MOErT/*/i2MOErA/*/i2MOErG/*/32MOErT/(SEQ ID NO: 179) or a salt thereof, wherein:

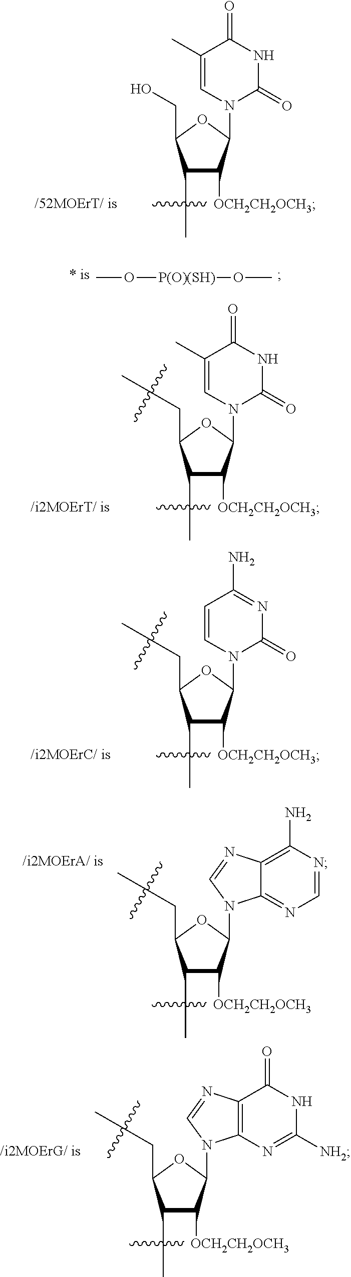

each of A, T and G is independently deoxyadenosine, thymidine, and deoxy guanosine, respectively;

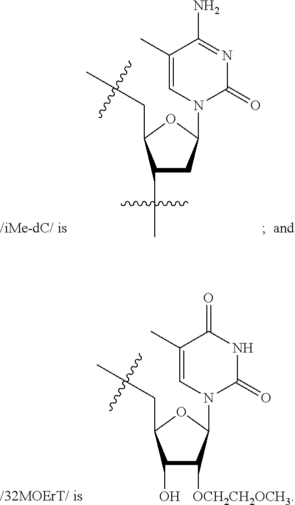

52. An oligonucleotide having the structure of:
   /52MOErG/*/i2MOErT/*/i2MOErT/*/i2MOErC/*/i2MOErC/*A*G*/iMe-dC/*T*T*G*G*G*/iMe-dC/*A*/i2MOErG/*/i2MOErT/*/i2MOErT/*/i2MOErG/*/32MOErT/ (SEQ ID NO: 117 or a salt thereof, wherein:

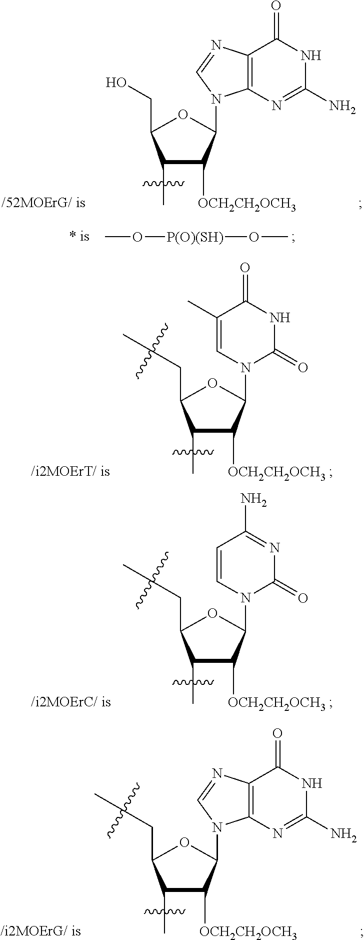

each of A, T and G is independently deoxyadenosine, thymidine, and deoxyguanosine, respectively;

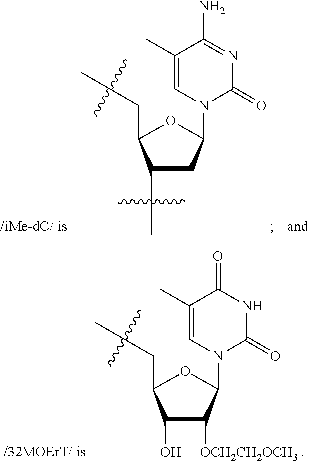

53. An oligonucleotide having the structure of
   /52MOErG/*/i2MOErG/*/i2MOErA/*/i2MOErA/*/i2MOErG/*/iMe-dC/*T*T*A*G*T*/iMe-dC/*/iMe-dC/*T*T*/i2MOErG/*/i2MOErG/*/i2MOErC/*/i2MOErT/*/32MOErG/ (SEQ ID NO: 138) or a salt thereof, wherein:

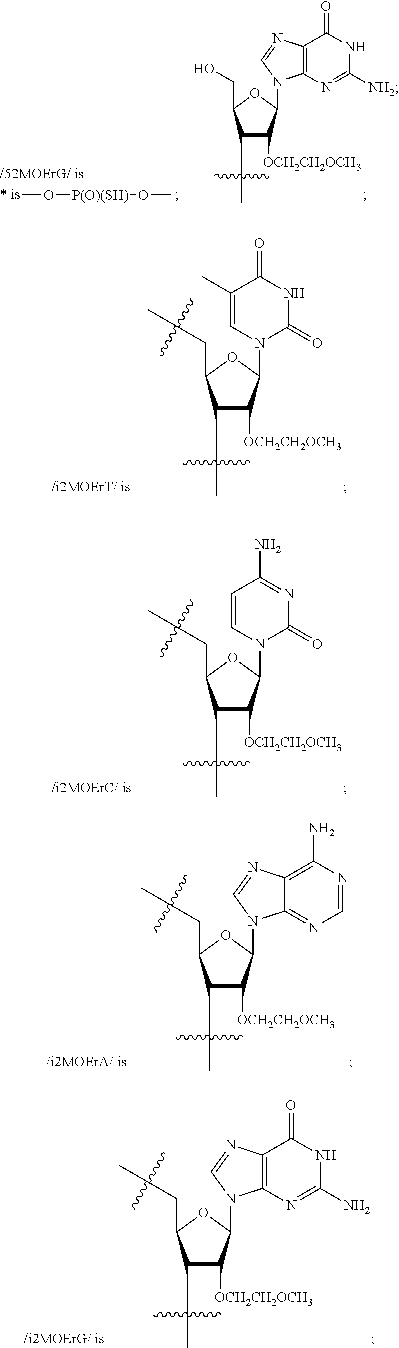

each of A, T and G is independently deoxyadenosine, thymidine, and deoxyguanosine, respectively;

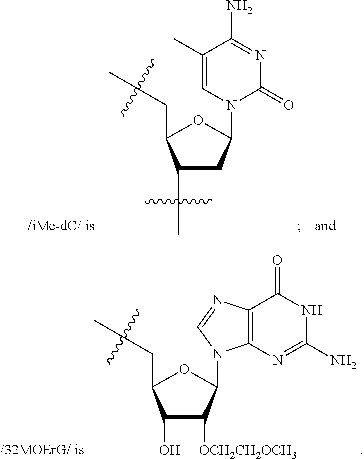

54. The oligonucleotide of any one of the preceding Embodiments, wherein the oligonucleotide is a pharmaceutically acceptable salt.
55. The oligonucleotide of any one of the preceding Embodiments, wherein the oligonucleotide is a sodium salt.
56. A composition comprising an oligonucleotide of any one of the preceding Embodiments and one or more diastereomers of the oligonucleotide with respect to chiral linkage phosphorus.
57. A composition comprising:
   an oligonucleotide or a salt thereof, and
   one or more diastereomers of the oligonucleotide with respect to chiral linkage phosphorus, or one or more salts of the diastereomers,
   wherein the oligonucleotide is
   /52MOErA/*/i2MOErT/*/i2MOErC/*/i2MOErA/*/i2MOErG/*T*T*T*/iMe-dC/*T*G*T*A*G*G*/i2MOErC/*/i2MOErT/*/i2MOErT/*/i2MOErC/*/32MOErC/ (SEQ ID NO: 116), wherein:

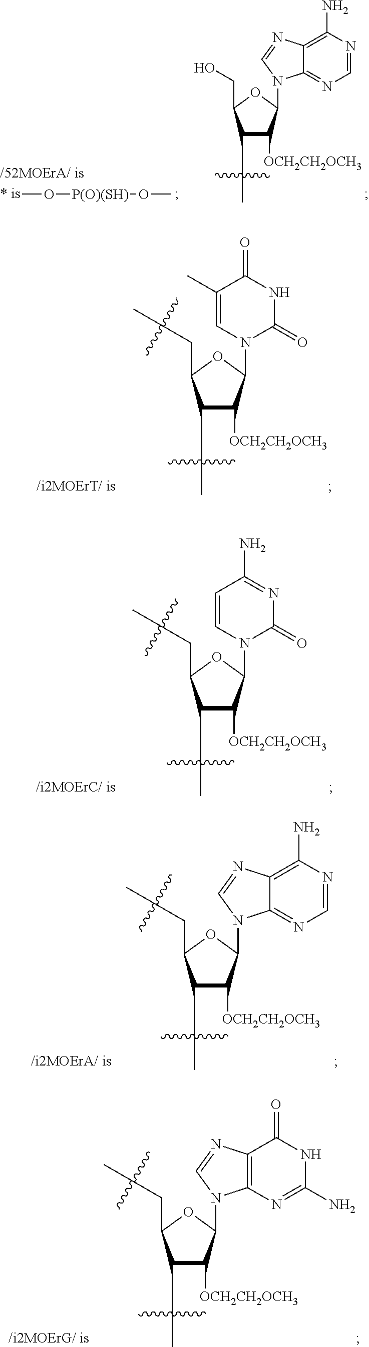

each of A, T and G is independently deoxyadenosine, thymidine, and deoxyguanosine, respectively;

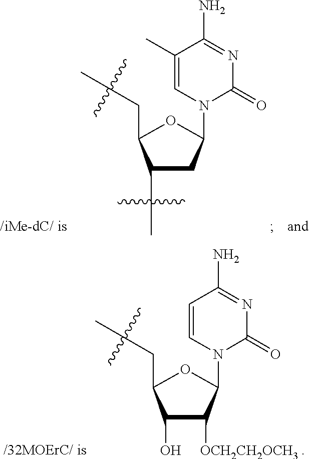

58. A composition comprising:
   an oligonucleotide or a salt thereof, and
   one or more diastereomers of the oligonucleotide with respect to chiral linkage phosphorus, or one or more salts of the diastereomers,
   wherein the oligonucleotide is
   /52MOErG/*/i2MOErG/*/i2MOErC/*/i2MOErA/*/i2MOErT/*A*/iMe-dC/*T*G*G*T*T*/iMe-dC/*A*G*/i2MOErT/*/i2MOErT/*/i2MOErG/*/i2MOErA/*/32MOErT/ (SEQ ID NO: 141), wherein:

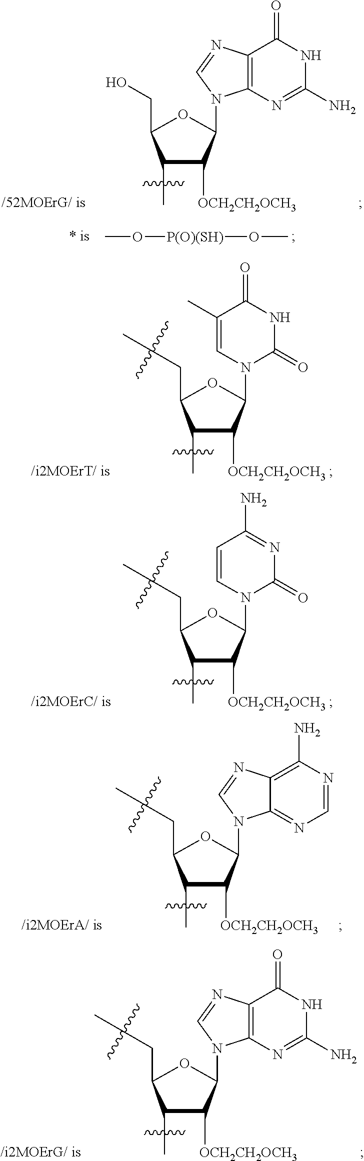

each of A, T md G is independently deoxyadenosine, thymidine, and deoxyguanosine, respectively:

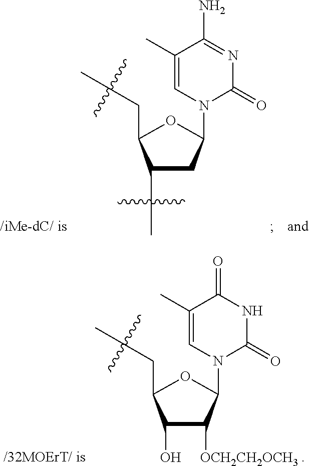

59. A composition comprising:
   an oligonucleotide or a salt thereof, and
   one or more diastereomers of the oligonucleotide with respect to chiral linkage phosphorus, or one or more salts of the diastereomers,
   wherein the oligonucleotide is
   /52MOErG/*/i2MOErC/*/i2MOErT/*/i2MOErC/*/i2MOErA/*G*G*T*/iMe-dC/*A*G*G*/iMe-dC/*A*G*/i2MOErT/*/i2MOErG/*/i2MOErG/*/i2MOErT/*/32MOErT/ (SEQ ID NO: 173, wherein:

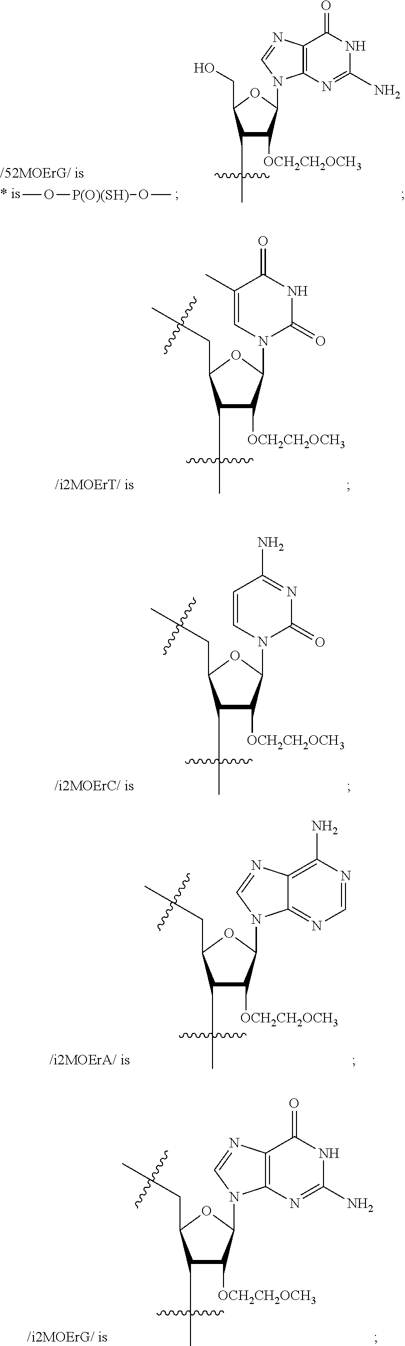

each of A, T and G is independently deoxyadenosine, thymidine, and deoxyguanosine, respectively;

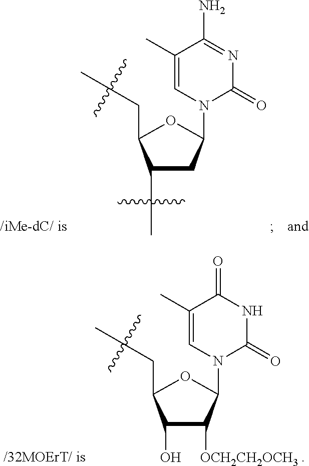

60. A composition comprising:
   an oligonucleotide or a salt thereof, and
   one or more diastereomers of the oligonucleotide with respect to chiral linkage phosphorus, or one or more salts of the diastereomers,
   wherein the oligonucleotide is
   /52MOErT/*/i2MOErC/*/i2MOErC/*/i2MOErA/*/i2MOErG/*/iMe-dC/*T*/iMe-dC/*T*G*T*G*/iMe-dC/*/iMe-dC/*T*/i2MOErC/*/i2MOErT/*/i2MOErA/*/i2MOErG/*/32MOErT/ (SEQ ID NO: 179), wherein:

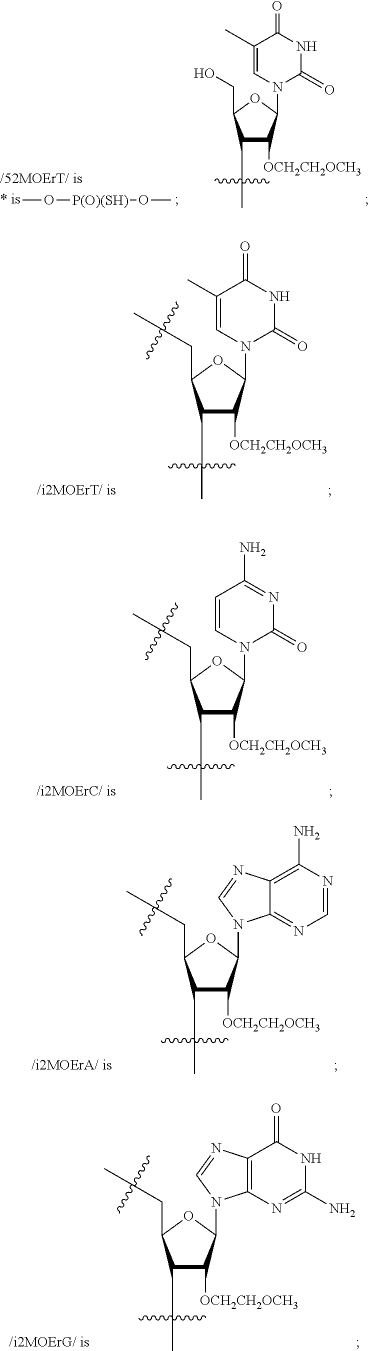

each of A, T and G is independently deoxyadenosine, thymidine, and deoxyguanosine, respectively:

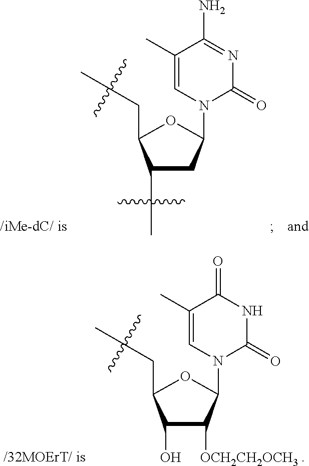

61. A composition comprising:
   an oligonucleotide or a salt thereof, and
   one or more diastereomers of the oligonucleotide with respect to chiral linkage phosphorus, or one or more salts of the diastereomers,
   wherein the oligonucleotide is
   /52MOErG/*/i2MOErT/*/i2MOErT/*/i2MOErC/*/i2MOErC/*A*G*/iMe-dC/*T*T*G*G*G*/iMe-dC/*A*/i2MOErG/*/i2MOErT/*/i2MOErT/*/i2MOErG/*/32MOErT/(SEQ ID NO: 117), wherein:

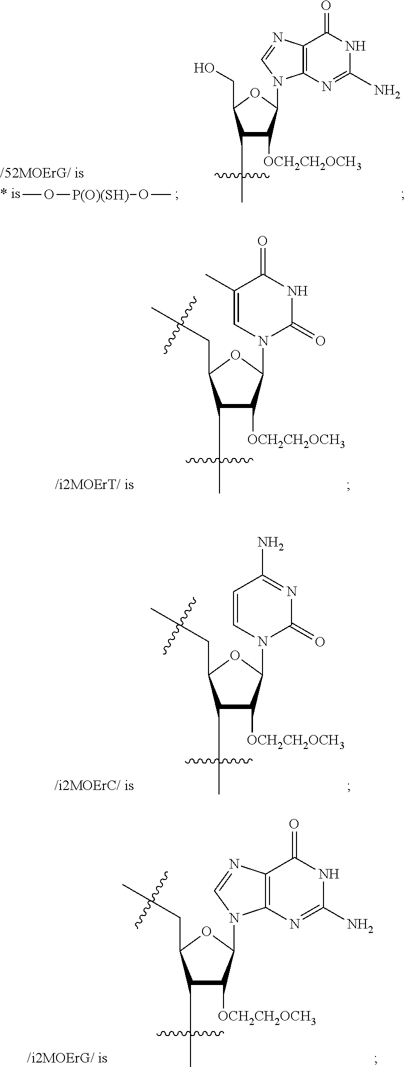

each of A, T and G is independently deoxyadenosine, thymidine, and deoxyguanosine, respectively;

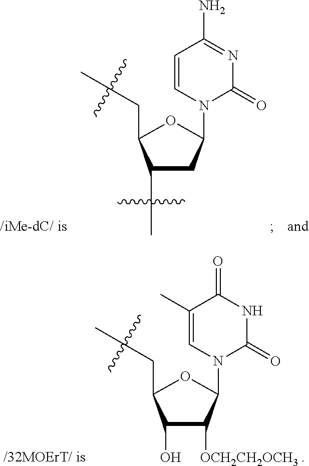

62. A composition comprising:
   an oligonucleotide or a salt thereof, and
   one or more diastereomers of the oligonucleotide with respect to chiral linkage phosphorus, or one or more salts of the diastereomers,
   wherein the oligonucleotide is
   /52MOErG/*/i2MOErG/*/i2MOErA/*/i2MOErA/*/i2MOErG/*/iMe-dC/*T*T*A*G*T*/iMe-dC/*/iMe-dC/*T*T*/i2MOErG/*/i2MOErG/*/i2MOErC/*/i2MOErT/*/32MOErG/ (SEQ ID NO: 138), wherein:

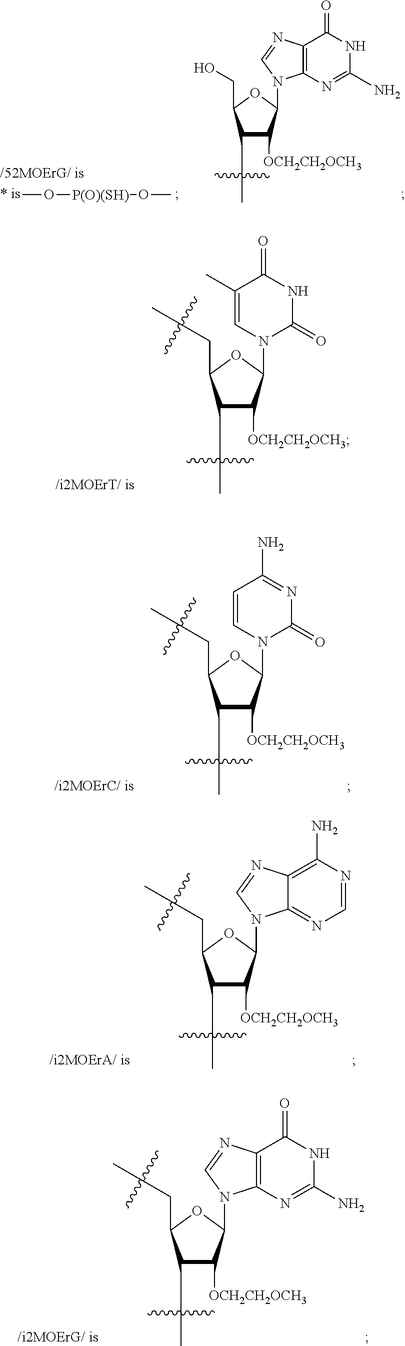

each of A, T and G is independently deoxyadenosine, thymidine, and deoxyguanosine, respectively:

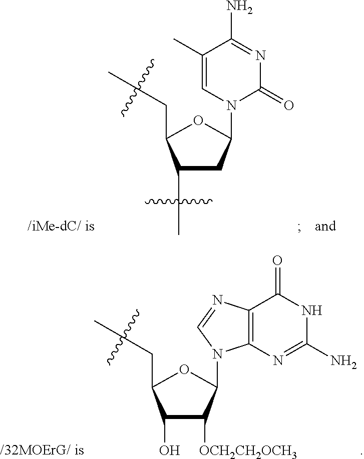

63. A composition comprising:
   an oligonucleotide or a salt thereof, and
   one or more diastereomers of the oligonucleotide with respect to chiral linkage phosphorus, or one or more salts of the diastereomers,
   wherein the oligonucleotide is
   /52MOErG/*/i2MOErA/*/i2MOErG/*/i2MOErA/*/i2MOErG/*/iMe-dC/*/iMe-dC/*T*T*T*T*T*G*/iMe-dC/*A*/i2MOErG/*/i2MOErA/*/i2MOErG/*/i2MOErC/*/32MOErT/(SEQ ID NO: 114), wherein:

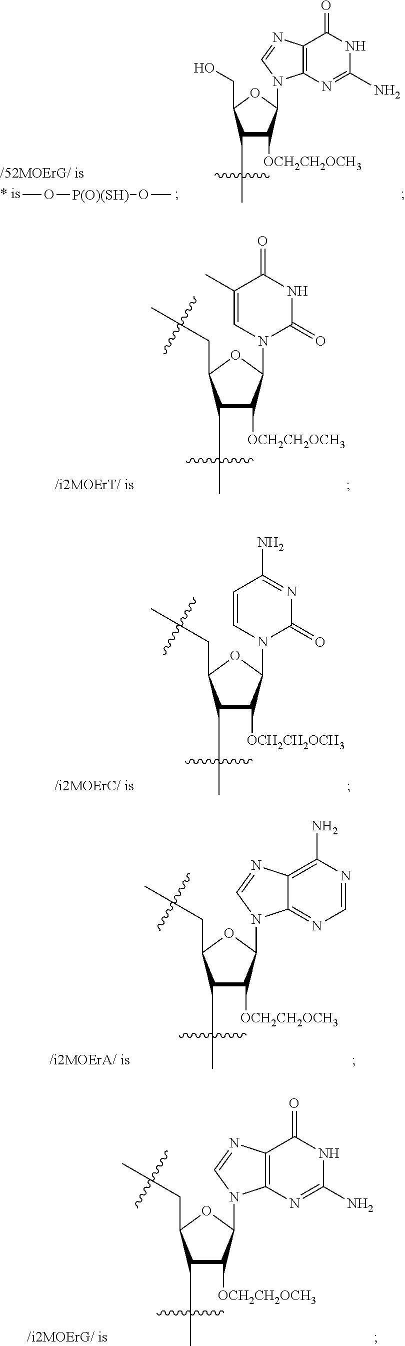

each of A, T and G is independently deoxyadenosine thymidine, and deoxyguanosine, respectively;

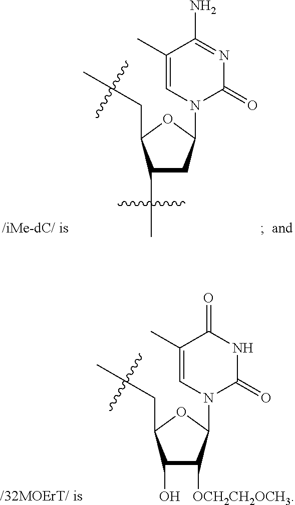

64. The composition of any one of Embodiments 56-63, wherein for each chiral linkage phosphorus, the percentage of the Rp configuration is independently about 20%-80%.
65. The composition of any one of Embodiments 56-63, wherein for each chiral linkage phosphorus, the percentage of the Rp configuration is independently about 30%-70%.
66. The composition of any one of Embodiments 56-63, wherein for each chiral linkage phosphorus, the percentage of the Rp configuration is independently about 40%-60%.
67. The composition of any one of Embodiments 56-63, wherein for each chiral linkage phosphorus, the percentage of the Rp configuration is independently about 45%-55%.
68. The composition of any one of Embodiments 56-63, wherein for each chiral linkage phosphorus, the percentage of the Rp configuration is independently about 50%.
69. The composition of any one of Embodiments 56-68, wherein the composition comprises a salt of the oligonucleotide, and one or more salts of one or more diastereomers.
70. The composition of any one of Embodiments 56-69, wherein the composition is a pharmaceutical composition, and further comprising a pharmaceutically acceptable carrier.
71. The composition of any one of Embodiments 56-70, wherein the composition comprises a pharmaceutically acceptable salt of the oligonucleotide, one or more pharmaceutically acceptable salts of one or more diastereomers, and a pharmaceutically acceptable carrier.
72. A pharmaceutical composition comprising an oligonucleotide of any one of the preceding Embodiments and a pharmaceutically acceptable carrier.
73. The composition of Embodiment 72, wherein the composition comprises one or more pharmaceutically acceptable salts of an oligonucleotide.
74. The composition of any one of Embodiments 56-73, wherein the composition is a liquid composition.
75. The composition of any one of Embodiments 70-74, wherein a pharmaceutically acceptable carrier is a buffer.
76. The composition of any one of Embodiments 70-74, wherein a pharmaceutically acceptable carrier is a buffered saline.
77. The composition of any one of Embodiments 70-74, wherein a pharmaceutically acceptable carrier is artificial cerebrospinal fluid.
78. A method for reducing level of calpain-2 mRNA in a system, comprising administering or delivering to the system an effective amount of an oligonucleotide or composition of any one of the preceding Embodiments.
79. A method for reducing level of calpain-2 polypeptide in a system, comprising administering or delivering to the system an effective amount of an oligonucleotide or composition of any one of the preceding Embodiments.
80. A method for reducing level of calpain-2 activity in a system, comprising administering or delivering to the system an effective amount of an oligonucleotide or composition of any one of the preceding Embodiments.
81. The method of any one of Embodiments 78-80, wherein the system expresses calpain-2 mRNA.
82. The method of any one of Embodiments 78-81, wherein the system is or comprises a cell.
83. The method of any one of Embodiments 78-81, wherein the system is or comprises a tissue.
84. The method of any one of Embodiments 78-81, wherein the system is or comprises an organ.
85. The method of any one of Embodiments 78-81, wherein the system is or comprises brain or a portion thereof.
86. The method of any one of Embodiments 78-81, wherein the system is or comprises an organism.
87. The method of any one of Embodiments 78-81, wherein the system is a subject.
88. The method of any one of Embodiments 78-81, wherein the system is a human.
89. The method of any one of Embodiments 78-88, wherein level of calpain-2 mRNA in the system is reduced by about or at least about 50%, 55%, 60%, 65%, 70%, 75%, 80%, 85%, 90%, 95%, 96%, 97%, 98% or 99% compared to absence of the oligonucleotide or composition.
90. The method of any one of Embodiments 78-88, wherein level of calpain-2 mRNA in the system is reduced by about or at least about 50% compared to absence of the oligonucleotide or composition.
91. The method of any one of Embodiments 78-88, wherein level of calpain-2 mRNA in the system is reduced by about or at least about 60% compared to absence of the oligonucleotide or composition.
92. The method of any one of Embodiments 78-88, wherein level of calpain-2 mRNA in the system is reduced by about or at least about 65% compared to absence of the oligonucleotide or composition.
93. The method of any one of Embodiments 78-88, wherein level of calpain-2 mRNA in the system is reduced by about or at least about 70% compared to absence of the oligonucleotide or composition.
94. The method of any one of Embodiments 78-88, wherein level of calpain-2 mRNA in the system is reduced by about or at least about 75% compared to absence of the oligonucleotide or composition.
95. The method of any one of Embodiments 78-88, wherein level of calpain-2 mRNA in the system is reduced by about or at least about 80% compared to absence of the oligonucleotide or composition.
96. The method of any one of Embodiments 78-88, wherein level of calpain-2 mRNA in the system is reduced by about or at least about 85% compared to absence of the oligonucleotide or composition.
97. The method of any one of Embodiments 78-96, wherein level of calpain-2 polypeptide in the system is reduced by about or at least about 10%, 15%, 20%, 25%, 30%, 35%, 40%, 45%, 50%, 55%, 60%, 65% or 70% compared to absence of the oligonucleotide or composition.
98. The method of any one of Embodiments 78-96, wherein level of calpain-2 polypeptide in the system is reduced by about or at least about 20% compared to absence of the oligonucleotide or composition.
99. The method of any one of Embodiments 78-96, wherein level of calpain-2 polypeptide in the system is reduced by about or at least about 30% compared to absence of the oligonucleotide or composition.
100. The method of any one of Embodiments 78-96, wherein level of calpain-2 polypeptide in the system is reduced by about or at least about 40% compared to absence of the oligonucleotide or composition.
101. The method of any one of Embodiments 78-96, wherein level of calpain-2 polypeptide in the system is reduced by about or at least about 50% compared to absence of the oligonucleotide or composition.
102. The method of any one of Embodiments 78-96, wherein level of calpain-2 polypeptide in the system is reduced by about or at least about 60% compared to absence of the oligonucleotide or composition.
103. The method of any one of Embodiments 78-102, wherein level of calpain-2 activity in the system is reduced by about or at least about 20%, 25%, 30%, 35%, 40%, 45%, 50%, 55%, 60%, 65%, 70%, 75% or 80% compared to absence of the oligonucleotide or composition
104. The method of any one of Embodiments 78-102, wherein level of calpain-2 activity in the system is reduced by about or at least about 20% compared to absence of the oligonucleotide or composition.
105. The method of any one of Embodiments 78-102, wherein level of calpain-2 activity in the system is reduced by about or at least about 30% compared to absence of the oligonucleotide or composition.
106. The method of any one of Embodiments 78-102, wherein level of calpain-2 activity in the system is reduced by about or at least about 40% compared to absence of the oligonucleotide or composition.
107. The method of any one of Embodiments 78-102, wherein level of calpain-2 activity in the system is reduced by about or at least about 50% compared to absence of the oligonucleotide or composition.
108. The method of any one of Embodiments 78-102, wherein level of calpain-2 activity in the system is reduced by about or at least about 60% compared to absence of the oligonucleotide or composition.
109. The method of any one of Embodiments 78-108, wherein level of calpain-2 mRNA in the system is reduced by about or at least about 50%, 55%, 60%, 65%, 70%, 75%, 80%, 85%, 90%, 95%, 96%, 97%, 98% or 99% compared to administering or delivering a reference oligonucleotide or composition.
110. The method of any one of Embodiments 78-108, wherein level of calpain-2 mRNA in the system is reduced by about or at least about 50% compared to administering or delivering a reference oligonucleotide or composition.
111. The method of any one of Embodiments 78-108, wherein level of calpain-2 mRNA in the system is reduced by about or at least about 60% compared to administering or delivering a reference oligonucleotide or composition.
112. The method of any one of Embodiments 78-108, wherein level of calpain-2 mRNA in the system is reduced by about or at least about 65% compared to administering or delivering a reference oligonucleotide or composition.
113. The method of any one of Embodiments 78-108, wherein level of calpain-2 mRNA in the system is reduced by about or at least about 70% compared to administering or delivering a reference oligonucleotide or composition.
114. The method of any one of Embodiments 78-108, wherein level of calpain-2 mRNA in the system is reduced by about or at least about 75% compared to administering or delivering a reference oligonucleotide or composition.
115. The method of any one of Embodiments 78-108, wherein level of calpain-2 mRNA in the system is reduced by about or at least about 80% compared to administering or delivering a reference oligonucleotide or composition.
116. The method of any one of Embodiments 78-108, wherein level of calpain-2 mRNA in the system is reduced by about or at least about 85% compared to administering or delivering a reference oligonucleotide or composition.
117. The method of any one of Embodiments 78-116, wherein level of calpain-2 polypeptide in the system is reduced by about or at least about 10%, 15%, 20%, 25%, 30%, 35%, 40%, 45%, 50%, 55%, 60%, 65% or 70% compared to administering or delivering a reference oligonucleotide composition.
118. The method of any one of Embodiments 78-116, wherein level of calpain-2 polypeptide in the system is reduced by about or at least about 20% compared to administering or delivering a reference oligonucleotide composition.
119. The method of any one of Embodiments 78-116, wherein level of calpain-2 polypeptide in the system is reduced by about or at least about 30% compared to administering or delivering a reference oligonucleotide composition.
120. The method of any one of Embodiments 78-116, wherein level of calpain-2 polypeptide in the system is reduced by about or at least about 40% compared to administering or delivering a reference oligonucleotide composition.
121. The method of any one of Embodiments 78-116, wherein level of calpain-2 polypeptide in the system is reduced by about or at least about 50% compared to administering or delivering a reference oligonucleotide composition.
122. The method of any one of Embodiments 78-116, wherein level of calpain-2 polypeptide in the system is reduced by about or at least about 60% compared to administering or delivering a reference oligonucleotide composition.
123. The method of any one of Embodiments 78-122, wherein level of calpain-2 activity in the system is reduced by about or at least about 20%, 25%, 30%, 35%, 40%, 45%, 50%, 55%, 60%, 65%, 70%, 75% or 80% compared to administering or delivering a reference oligonucleotide composition.
124. The method of any one of Embodiments 78-122, wherein level of calpain-2 activity in the system is reduced by about or at least about 20% compared to administering or delivering a reference oligonucleotide composition.
125. The method of any one of Embodiments 78-122, wherein level of calpain-2 activity in the system is reduced by about or at least about 30% compared to administering or delivering a reference oligonucleotide composition.
126. The method of any one of Embodiments 78-122, wherein level of calpain-2 activity in the system is reduced by about or at least about 40% compared to administering or delivering a reference oligonucleotide composition.
127. The method of any one of Embodiments 78-122, wherein level of calpain-2 activity in the system is reduced by about or at least about 50% compared to administering or delivering a reference oligonucleotide composition.
128. The method of any one of Embodiments 78-122, wherein level of calpain-2 activity in the system is reduced by about or at least about 60% compared to administering or delivering a reference oligonucleotide composition.
129. The method of any one of Embodiments 109-128, wherein the reference oligonucleotide does not target calpain-2 or the reference composition comprises no oligonucleotides targeting calpain-2.
130. The method of any one of Embodiments 109-128, wherein the reference oligonucleotide targets TUG1 or the reference composition comprises oligonucleotides targeting TUG1.
131. The method of any one of Embodiments 89-130, wherein the reduction is assessed at or after about 1, 2, 3, 4, 5, 6, 7, 8, 9, 10, 11, 12, 13, 14, 15, 16, 17, 18, 19, 20, 21, 22, 23, 24 or 25 days, or about 1, 2, 3 or 4 weeks following administering or delivering the oligonucleotide or composition.
132. The method of any one of Embodiments 89-131, wherein the reduction is assessed at about D3 following administering or delivering the oligonucleotide or composition.
133. The method of any one of Embodiments 89-132, wherein the reduction is assessed at about D7 following administering or delivering the oligonucleotide or composition.
134. The method of any one of Embodiments 89-133, wherein the reduction is assessed at about D10 following administering or delivering the oligonucleotide or composition.
135. The method of any one of Embodiments 89-134, wherein the reduction is assessed at about D14 following administering or delivering the oligonucleotide or composition.
136. The method of any one of Embodiments 89-135, wherein the reduction is assessed at about D21 following administering or delivering the oligonucleotide or composition.
137. The method of any one of Embodiments 89-136, wherein the reduction is assessed at or after about 1, 2, 3, 4, 5, 6, 7, 8, 9, 10, 11, 12, 13, 14, 15, 16, 17, 18, 19, 20, 21, 22, 23, 24 or 25 days, or about 1, 2, 3 or 4 weeks following removal or washout of the oligonucleotide or composition.
138. The method of any one of Embodiments 89-137, wherein the reduction is assessed at about D3 following removal or washout of the oligonucleotide or composition.
139. The method of any one of Embodiments 89-138, wherein the reduction is assessed at about D7 following removal or washout of the oligonucleotide or composition.
140. The method of any one of Embodiments 89-139, wherein the reduction is assessed at about D10 following removal or washout of the oligonucleotide or composition.
141. The method of any one of Embodiments 89-140, wherein the reduction is assessed at about D14 following removal or washout of the oligonucleotide or composition.
142. The method of any one of Embodiments 89-141, wherein the reduction is assessed at about D21 following removal or washout of the oligonucleotide or composition.
143. The method of any one of Embodiments 89-142, wherein the reduction is assessed in iPSC-derived motor neurons with oligonucleotide concentration at about 20 uM with gymnotic delivery.
144. A method for preventing or treating a condition, disorder or disease, comprising administering or delivering to a subject susceptible thereto an effective amount of an oligonucleotide or composition of any one of Embodiments 1-77.
145. The method of Embodiment 144, wherein onset of the condition, disorder or disease is delayed.
146. The method of Embodiment 144, wherein onset of the condition, disorder or disease is prevented.
147. A method for treating a condition, disorder or disease, comprising administering or delivering to a subject suffering therefrom an effective amount of an oligonucleotide or composition of any one of Embodiments 1-77.
148. The method of Embodiment 147, wherein severity of a symptom of the condition, disorder or disease is reduced.
149. The method of any one of Embodiments 147-148, wherein one or more clinical assessment results of the subject are independently improved.
150. The method of any one of Embodiments 144-149, wherein a condition, disorder or disease is associated with Wallerian degeneration.
151. The method of any one of Embodiments 144-149, wherein a condition, disorder or disease is a neurodegenerative condition, disorder or disease.
152. The method of any one of Embodiments 144-149, wherein a condition, disorder or disease is amyotmphic lateral sclerosis.
153. The method of any one of Embodiments 144-149, wherein a condition, disorder or disease is peripheral neuropathy.
154. The method of any one of Embodiments 144-149, wherein a condition, disorder or disease is peripheral neuropathy induced by chemotherapy.
155. The method of any one of Embodiments 144-149, wherein a condition, disorder or disease is Parkinson's disease.
156. The method of any one of Embodiments 144-149, wherein a condition, disorder or disease is Huntington's disease.
157. The method of any one of Embodiments 144-149, wherein a condition, disorder or disease is Alzheimer's disease.
158. The method of any one of Embodiments 144-149, wherein a condition, disorder or disease is frontotemporal dementia.
159. The method of any one of Embodiments 144-149, wherein a condition, disorder or disease is traumatic brain injury.
160. The method of any one of Embodiments 144-149, wherein a condition, disorder or disease is progressive supranuclear palsy.
161. The method of any one of Embodiments 144-149, wherein a condition, disorder or disease is corticobasal degeneration.
162. The method of any one of Embodiments 144-149, wherein a condition, disorder or disease is Wolfram Syndrome.
163. The method of any one of Embodiments 144-149, wherein a condition, disorder or disease is Friedreich's Ataxia.
164. The method of any one of Embodiments 144-149, wherein a condition, disorder or disease is multiple system atrophy.
165. The method of any one of Embodiments 144-149, wherein a condition, disorder or disease is spinal cerebellar ataxia.
166. The method of any one of Embodiments 144-149, wherein a condition, disorder or disease is spinal muscular atrophy.
167. The method of any one of Embodiments 144-149, wherein a condition, disorder or disease is Pick's Disease.
168. The method of any one of Embodiments 144-149, wherein a condition, disorder or disease is progressive motor atrophy.
169. The method of any one of Embodiments 144-149, wherein a condition, disorder or disease is stroke.
170. The method of any one of Embodiments 144-149, wherein a condition, disorder or disease is concussion.
171. The method of any one of Embodiments 144-149, wherein a condition, disorder or disease is intracerebral hemorrhage.
172. The method of any one of Embodiments 144-149, wherein a condition, disorder or disease is acute glaucoma.
173. The method of any one of Embodiments 144-149, wherein a condition, disorder or disease is seizure.
174. The method of any one of Embodiments 144-149, wherein a condition, disorder or disease is spinal cord injury.
175. The method of any one of Embodiments 78-174, wherein NF-L excretion is reduced.
176. A method for reducing NF-L excretion, comprising administering or delivering to a subject an effective amount of an oligonucleotide or composition of any one of Embodiments 1-77.
177. The method of any one of the preceding Embodiments, wherein neuritic degeneration is reduced.
178. A method for reducing neuritic degeneration, comprising administering or delivering to a subject an effective amount of an oligonucleotide or composition of any one of Embodiments 1-77.
179. The method of any one of Embodiments 175-178, wherein NF-L excretion and/or neuritic degeneration is toxicity-triggered.
180. The method of any one of Embodiments 144-179, wherein the oligonucleotide or composition is administered or delivered intrathecally.
181. The method of any one of Embodiments 144-179, wherein the oligonucleotide or composition is administered or delivered intravenously.

### EXAMPLES

Certain examples of provided technologies (compounds (oligonucleotides, reagents, etc.), compositions, methods (methods of preparation, use, assessment, etc.), etc.) were presented herein. Those skilled in the art appreciate that many technologies can be utilized to prepare and/or assess properties and/or activities of provided technologies in accordance with the present disclosure.

### Example 1. Various Oligonucleotides and Compositions can Reduce Calpain-2 Expression

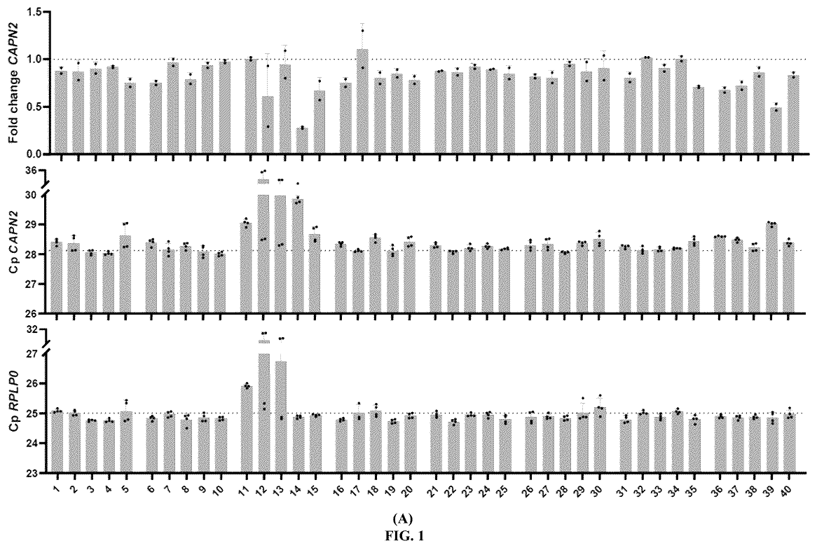

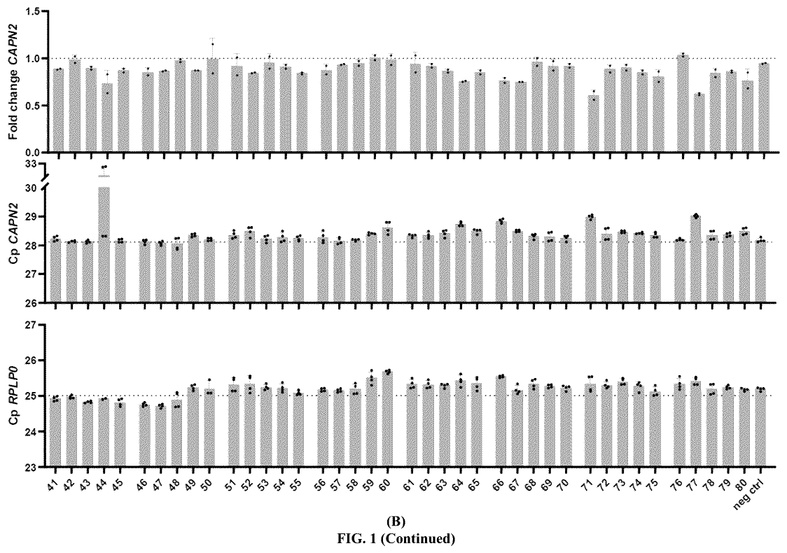

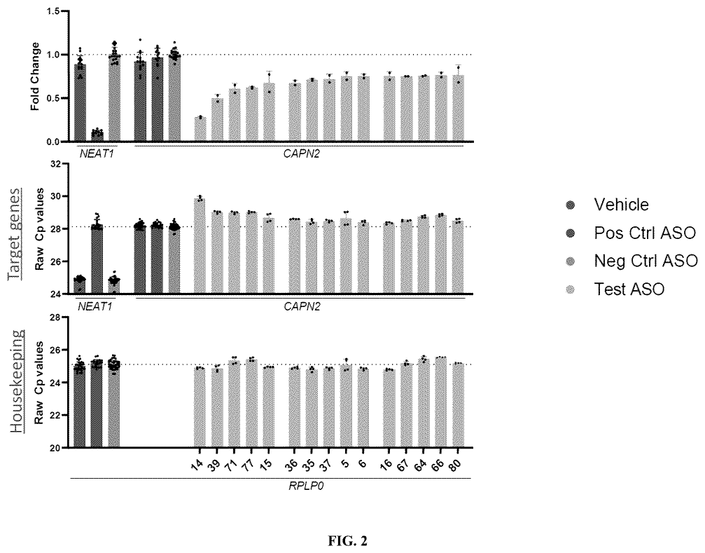

Oligonucleotides and compositions as described herein can provide in vitro knockdown of calpain-2 (CAPN2) transcript. In an assessment, human induced pluripotent stem cell (iPSC)-derived glutamatergic neurons (bit.bio ioGlutamatergic Neurons, Cat. No.: io1001) were seeded at 20,000 cells/well in a 96-well format. Following culturing of the cells for 4 days, various oligonucleotide compositions were added to the cells at a concentration of 5 μM in 5% TE buffer. Cells were incubated with the oligonucleotides for 48 hours to allow for gymnotic uptake and knockdown of CAPN2 expression. Following this incubation period, cells were lysed and RNA was collected (Thermo Fisher cat. #A25603). RNA was used in real time RT-qPCR to quantify fold-change of CAPN2 expression. In some embodiments, as shown in FIG. 1, various oligonucleotides provided in vitro knockdown of calpain-2 expression. As shown in FIG. 1, various oligonucleotides provided substantial in vitro knockdown of CAPN2 expression as compared to reference conditions. Negative control is /52MOErC/*/i2MOErC/*/i2MOErT/*/i2MOErA/*/i2MOErT/*A*G*G*A*/iMe-dC/*T*A*T*/iMe-dC/*/iMe-dC/*/i2MOErA/*/i2MOErG/*/i2MOErG/*/i2MOErA/*/32MOErA/ (SEQ ID NO: 183). See also data in FIG. 2 (positive control is <T*G*C*A*A*>G*T*C*T*G*A*C*G*C*C*<C*A*T*C*T> (SEQ ID NO: 184), negative control is <A*C*C*A*G*>T*G*C*A*T*T*C*A*T*T*<C*G*A*G*T> (SEQ ID NO: 185); all nucleosides are DNA nucleosides except those in < > which are 2′-MOE modified, and each * independently represents a phosphorothioate internucleotidic linkage).

### Example 2. Various Oligonucleotides and Compositions do not Display Obvious Cytotoxicity

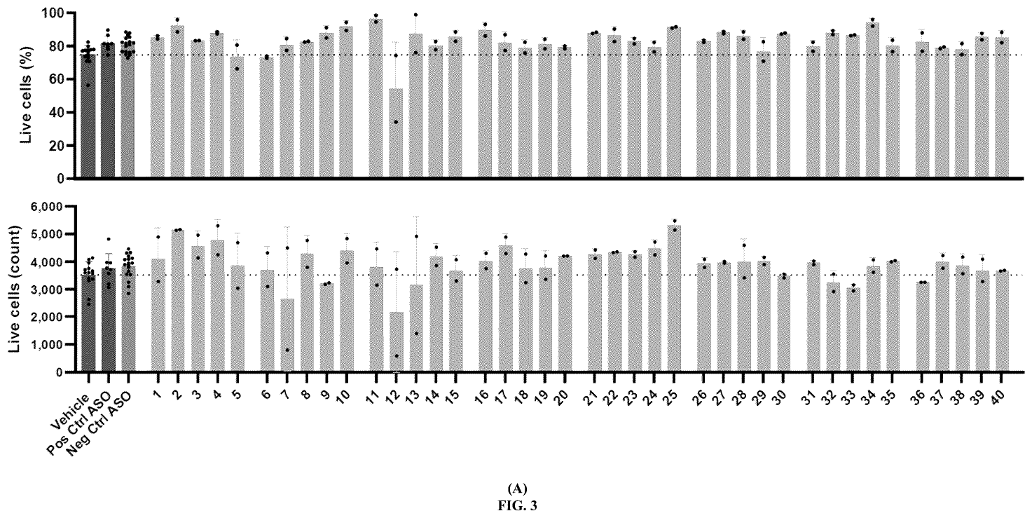

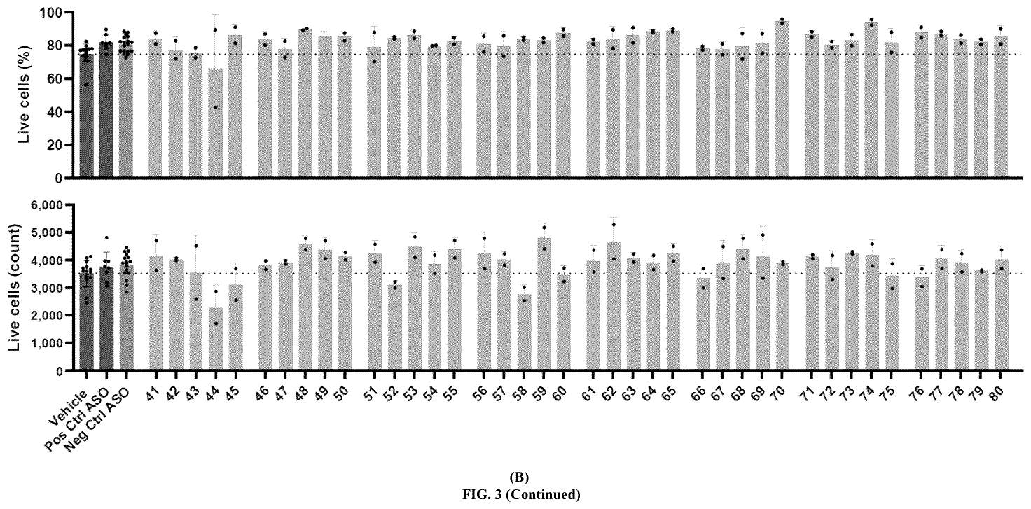

Among other things, the present disclosure provides oligonucleotides and compositions having low or no cytotoxicity. In an assessment, human iPSC-derived glutamatergic neurons (bit.bio ioGlutamatergic Neurons, Cat. No.: io1001) were seeded at 20,000 cells/well in a 96-well format. Following culturing of the cells for 4 days, various oligonucleotides were added to the cells at a concentration of 5 μM in 5% TE buffer. Cells were incubated with the oligonucleotides for 48 hours. Following this incubation period, cells underwent Hoechst staining (5 μg/mL). The number of live, dead, and total (live+dead) cells were counted and the percentage of live cells was calculated. As shown in FIG. 3, no significant change in the percentage of live cells were observed following treatment with various oligonucleotides as compared to vehicle treatment (TE buffer). Positive control is <T*G*C*A*A*>G*T*C*T*G*A*C*G*C*C*<C*A*T*C*T> (SEQ ID NO: 184) (targeting NEAT1), negative control is <A*C*C*A*G*>T*G*C*A*T*T*C*A*T*T*<C*G*A*G*T> (SEQ ID NO: 185 kJ all nucleosides are DNA nucleosides except those in < > which are 2′-MOE modified, and each * independently represents a phosphorothioate internucleotidic linkage. Staurosporine was utilized to induce toxicity in a separate well.

### Example 3. Various Oligonucleotides and Compositions can Provide Dose-Dependent Reduction of Calpain-2 Expression

Among other things, the present disclosure demonstrate that various provided oligonucleotide and compositions can provide dose-dependent reduction of calpain-2 mRNA levels. Results from an assessment are presented below as an example.

Cells: Human Bit.bio ioGlutamatergic neurons, seeded at 20,000 cells/well in 96-well format. Thawing, seeding and culturing according to supplier's instructions (coating: 0.01% PLO and 26 ng/cm2 laminin).

Oligonucleotide treatment: oligonucleotide compositions 14, 15, 36, 39, 71, 77 and TUG1 (negative control). Concentration range: 20 μM, 15 μM, 10 μM, 5 μM, 2.5 μM, 1.25 μM, 0.63 μM, 0.31 μM. Positive control (targeting NEAT1): oligonucleotide targeting NEAT1 (5 μM). Negative control: TUG1 (5 μM). Vehicle: 20% TE buffer without oligonucleotides (same TE buffer content for all conditions). Oligonucleotides were added to cells 4 days after seeding the cells (by gymnotic uptake, 48 hours incubation). Treatments were in duplicates on separate plates, except for negative control TUG1 oligonucleotides. Positive control: <T*G*C*A*A*>G*T*C*T*G*A*C*G*C*C*<C*A*T*C*T> (SEO ID NO: 184) (targeting NEAT1): negative control: <A*C*C*A*G*>T*G*C*A*T*T*C*A*T*T*<C*G*A*G*T> (SEQ ID NO: 185); all nucleosides are DNA nucleosides except those in < > which are 2′-MOE modified, and each * independently represents a phosphorothioate internucleotidic linkage.

Harvesting: Cells-to-Ct 1-Step TaqMan kit (Life technologies), according to supplier's instructions (cell lysis without separate RNA purification and one-step reverse transcription+real time PCR (real time RT-PCR). 30 μL/well lysis buffer. 2 days after treatment.

RT-qPCR 10% of Cells-to-Ct lysate samples (or water for non-template control; NTC). Single-plex reactions, in 384-well format, in technical duplicates. TaqMan Gene Expression Assay using LightCycler 480 equipment. (NEAT1 Taqman assay for control oligonucleotide samples only, CAPN2 and RPLP0 Taqman assays for control and test oligonucleotide samples).

| Primer-probe Set | Supplier | Assay ID |
|---|---|---|
| Human NEAT1 FAM | IDT | |
| (target; control samples) | | |
| Human CAPN2 FAM | Thermo Fisher | Hs00965097_m1 |
| (target) | | |
| Human RPLP0VIC_PL | Thermo Fisher | Hs00420895_gH |
| (housekeeping) | | |

Data analysis: Cp values were determined using the second derivative method (Lightcycler software). Fold-change gene expression was determined by ΔΔCp method (normalized to negative control oligonucleotide: TUG1 at 5 μM). For NEAT1 expression: ΔCp=Cp_{NEAT1}−Cp_{RPLP0}; ΔΔCp=ΔCp_{NEAT1}−ΔCp_{TUG1}; Fold change of NEAT1 expression=2^{ΔΔCp}. For CAPN2 expression: ΔCp=_{CAPN2}−Cp_{RPLP0}; ΔΔCp=ΔCp_{CAPN2}−ΔCp_{TUG1}; fold change of CAPN2 expression=2^{-ΔΔCp}.

A strong separation of positive and negative control oligonucleotide was observed. There was no marked intra- or inter-plate variability between positive and negative controls. No plate positional effects were observed based on control sample performance (vehicle, positive and negative controls), and no clear plate drift was observed between plates tested. No effects of control treatment on calpain-2 and/or RPLP0 expression were observed when compared to vehicle treatment (fold changes and/or Cp values). Concordance between biological replicates was observed.

As demonstrated in the table below, concentration-dependent increase in calpain-2 knockdown was observed for various provided oligonucleotide compositions. No effects on expression of RPLP0 were observed. The negative control targeting TUG1 did not show a concentration-dependent effect on calpain-2 expression. No apparent cell toxicity was observed at highest oligonucleotide concentrations assessed (20 μM) as well as lower concentrations.

| | % Knockdown of calpain-2 mRNA | | | | | |
|---|---|---|---|---|---|---|
| Conc. | 14 | 39 | 71 | 77 | 36 | 15 |
| 20 μM | 74 | 68 | 55.5 | 57 | 42 | 27.5 |
| 15 μM | 70 | 69 | 56 | 50.5 | 32.5 | 21 |
| 10 μM | 64.5 | 61.5 | 48.5 | 45 | 31 | -2 |
| 5 μM | 58.5 | 51.5 | 34 | 38.5 | 30.5 | 18.5 |
| 2.5 μM | 45.5 | 45 | 38 | 27 | 27 | 5.5 |
| 1.25 μM | 43.5 | 26.5 | 23 | 22.5 | 20.5 | 9 |
| 0.63 μM | 27 | 21.5 | 19.5 | 23 | 8 | 12.5 |
| 0.32 μM | 21.5 | 11 | 8.5 | 7.5 | 13 | 5 |

### Example 4. Various Oligonucleotides and Compositions can Provide Dose-Dependent Reduction of Calpain-2 mRNA Levels

Among other things, the present disclosure demonstrates that various provided oligonucleotide and compositions can provide dose-dependent reduction of calpain-2 mRNA levels. Results from an assessment are presented below as an example.

Cells: iCell Motor Neurons, seeded at 32,000 cells/well in 96-well format. Thawing, seeding and culturing can be performed according to supplier's instructions (e.g., coating: 0.07% PEI+10 μg/ml Cultrex 3D mouse laminin; medium; complete maintenance medium with 20 mM DAPT for day 0 through day 6, complete maintenance medium without DAPT for day 7 through day 10). Cell viability was examined at day 10 by staining with Hoechst (5 μg/ml) and imaging to count nuclei.

Oligonucleotide treatment: Oligonucleotide compositions 14, 39, and TUG1 (negative control). Concentration range: 20 μM, 6.329 μM, 2.003 μM, 0.634 μM, 0.201 μM, 0.063 μM, 0.020 μM, 0.006 μM. Positive control (targeting NEAT1): oligonucleotide targeting NEAT1 (20 μM). Vehicle: 2% (v/v) TE buffer without oligonucleotides. Oligonucleotides were added to cells on day 7 after seeding the cells and refreshed on day 9 (by gymnotic uptake, 72 hours total incubation). Treatments were in triplicates on separate plates, except for positive control NEAT1 oligonucleotides and vehicle, which both had 6 replicates per plate. Positive control: <T*G*C*A*A*>G*T*C*T*G*A*C*G*C*C*<C*A*T*C*T> (SEQ ID NO: 184) (targeting NEAT1); negative control: <A*C*C*A*G*>T*G*C*A*T*T*C*A*T*T*<C*G*A*G*T> (SEQ ID NO: 185); all nucleosides are DNA nucleosides except those in < > which are 2′-MOE modified, and each * independently represents a phosphorothioate internucleotidic linkage.

Harvesting: Cells can be harvested using Cells-to-Ct 1-Step TaqMan kit (Life Technologies), according to supplier's instructions (cell lysis without separate RNA purification and one-step reverse transcription+real time PCR (real time RT-PCR). 25 μL/well lysis buffer. 3 days after treatment.

RT-qPCR: 15% of Cells-to-Ct lysate samples (or water for non-template control; NTC). Single-plex reactions, in 384-well format, in technical duplicates. TaqMan Gene Expression Assay using LightCycler 480 equipment. (NEAT1 Taqman assay for control oligonucleotide samples only. CAPN2 and RPLP0 Taqman assays for control and test oligonucleotide samples).

| Primer-probe Set | Supplier | Assay ID |
|---|---|---|
| Human NEAT1 FAM | IDT | |
| (target; control samples) | | |
| Human CAPN2 FAM | Thermo Fisher | Hs00965097_m1 |
| (target) | | |
| Human RPLP0VIC_PL | Thermo Fisher | Hs00420895_gH |
| (housekeeping) | | |

Data analysis: Cp values were determined using the second derivative method (Lightcycler software). % knockdown of gene expression was determined by Pfaffl method to account for primer efficiency mismatch: RQ=(E_{target})^{ΔCt(target)}/(E_{reference})^{ΔCt(reference)}) (normalized to negative control: TUG1), wherein ΔCt (target)=Ct (target gene in calibrator)−Ct (target gene in tests) and ΔCt (reference)=Ct (reference gene in calibrator)−Ct (reference gene in tests).

Treatment with positive control oligonucleotide targeting NEAT1 resulted in specific knockdown of NEAT1, and treatment with negative control oligonucleotide targeting TUG1 resulted in slightly lower NEAT1 Cp values as compared to vehicle. Treatment with positive control oligonucleotide targeting NEAT1 or negative control oligonucleotide targeting TUG1 did not markedly affect RPLP0 or CAPN2 Cp values when compared to vehicle treatment. No appreciable plate-to-plate variation was observed. Significant variation in cell viability was observed by brightfield imaging throughout experiment which resulted in variation in sample input for RT-qPCR and exclusion of some data points. Low variation between most technical duplicates was observed. NTC displayed no amplification, indicating absence of contamination. No substantial variation in total cell counts was observed between plates. No apparent oligonucleotide-specific cell toxicity was observed at any concentration.

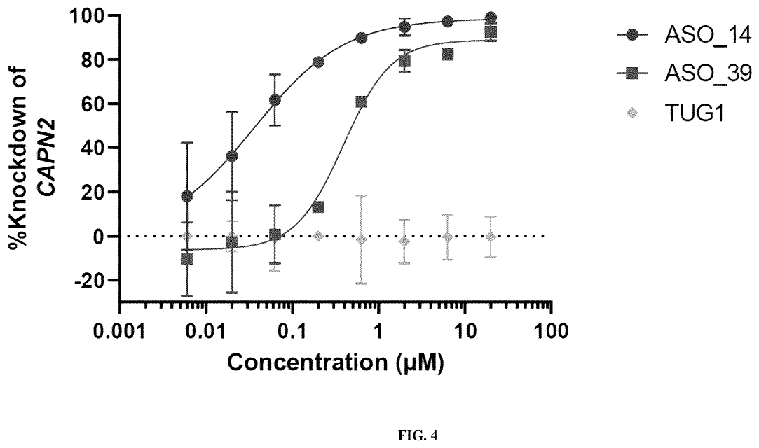

As demonstrated in FIG. 4 and the table below, concentration-dependent increase in CAPN2 knockdown was observed. Oligonucleotide 14 can provide about 96% knock-down of CAPN2 at 20 uM at 72 hr. No significant effects on expression of RPLP0 were observed. The negative control oligonucleotide targeting TUG1 did not show a concentration-dependent effect on CAPN2 mRNA levels. Nonlinear regression (variable Hill slope) was used to convert EC50 values to pEC50 values.

| Oligonucleotide | pEC50 | Goodness of Fit (R²) |
|---|---|---|
| 14 | 7.44 | 0.9048 |
| 39 | 6.41 | 0.9361 |

### Example 5. Various Oligonucleotides and Compositions can Provide Neuroprotective Effects

Among other things, the present disclosure demonstrate that various provided oligonucleotide and compositions can provide neuroprotective effects, e.g., reduced Neurofilament-L (NF-L) excretion following exposure to toxic triggers. Results from an assessment are presented below as an example.

Cells: iCell Motor Neurons, seeded at 32,000 cells/well in 96-well format. Thawing, seeding and culturing can be performed according to supplier's instructions (e.g., coating: 0.07% PEI+10 μg/ml Cultrex 3D mouse laminin; medium; complete maintenance medium with 20 mM DAPT for day 0 through day 6, complete maintenance medium without DAPT for day 7 through day 10).

Oligonucleotide treatment: Oligonucleotide composition 14 and TUG1 (negative control). Concentration: 20 μM. Vehicle: 2% (v/v) TE buffer without oligonucleotides (same TE buffer content for all conditions). Oligonucleotides were added to cells on day 7 after seeding the cells and refreshed on day 9 (by gymnotic uptake, 72 hours total incubation). Treatments were performed in replicate (in some embodiments. N=4 per oligonucleotide treatment per toxicity trigger). Negative control: <A*C*C*A*G*>T*G*C*A*T*T*C*A*T*T*<C*G*A*G*T> (SEQ ID NO: 185) (targeting TUG1); all nucleosides are DNA nucleosides except those in < > which are 2′-MOE modified, and each * independently represents a phosphorothioate internucleotidic linkage.

Toxicity triggers: Various toxicity triggers were added on day 9. Toxicity triggers used include vincristine (1.5 nM, 3 nM, 6 nM), rotenone (5 μM, 10 μM, 15 μM), and colchicine (10 nM, 100 nM, 1000 nM).

Neurofilament-L assay: Supernatant from cells was harvested on day 10 at 24 hours after addition of toxicity triggers. Concentration of Neurofilament-L (NF-L) was determined using R-PLEX Human Neurofilament-L Assay kit (Meso Scale Discovery) according to supplier instructions.

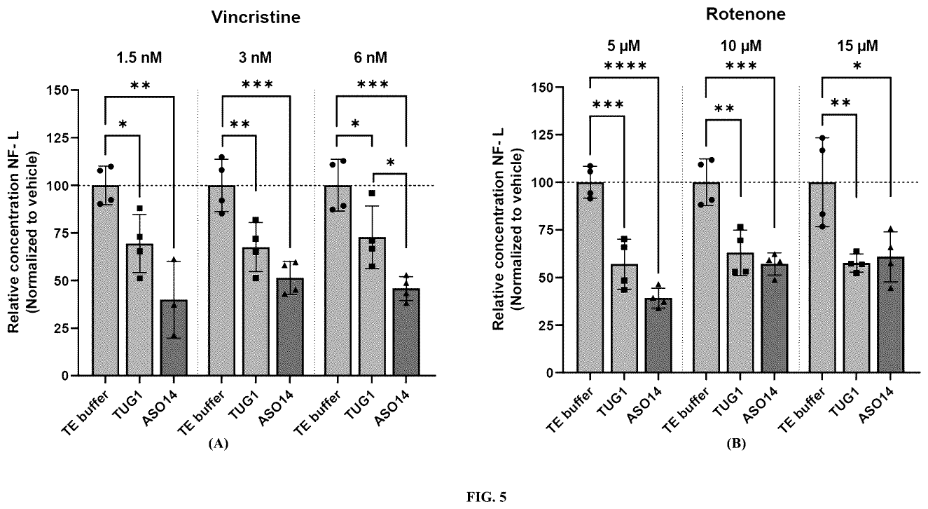

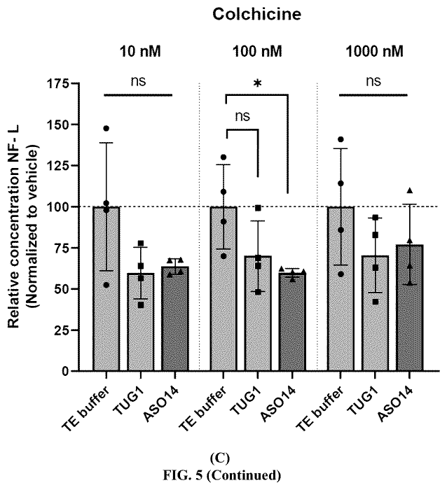

As shown in FIG. 5, pre-treatment with oligonucleotide composition 14 reduced toxicity trigger-induced NF-L excretion as compared to vehicle. A significant reduction of NF-L excretion following exposure to vincristine (at 1.5 nM, 3 nM, or 6 nM; FIG. 5, (A)), rotenone (at 5 μM, 10 μM, or M; FIG. 5, (B)), or colchicine (100 nM; FIG. 5, (C)) as compared to vehicle was observed with pre-treatment with oligonucleotide composition 14. The negative control (targeting TUG1) oligonucleotide composition also reduced toxicity trigger-induced NF-L excretion as compared to vehicle. Without the intention to be limited by any theory, the observed reduction with the TUG1-targeted oligonucleotide composition may be due to a calpain-independent effect. Further, a greater reduction in NF-L excretion was observed with oligonucleotide composition 14 as compared to the TUG1-targeted oligonucleotide composition in various cases. In some embodiments, NF-L analysis by MSD showed an observable assay window of oligonucleotide composition 14 over TUG1.

When assessed in a neuritic degeneration assay, it appeared that oligonucleotide composition 14 partially prevented trigger induced neuritic degeneration, e.g., as measured by tubulin immunoreactivity area following exposure to toxicity triggers. In some embodiments, certain effects were also observed for TUG1-targeted oligonucleotide composition.

### Example 6. Provided Technologies can Reduce Reduction of Calpain-2 mRNA and Protein Levels

Among other things, the present disclosure demonstrates that various provided oligonucleotide and compositions can provide reduction of calpain-2 mRNA and protein levels, in some instances, for days, weeks or longer. Results from certain assessments are presented below as an example. Those skilled in the art appreciate that other technologies can also be utilized to assess provided technologies in accordance with the present disclosure to confirm technical effects, benefits, advantages, etc. of provided technologies.

Cells: iCell Motor Neurons, seeded at 32,000 cells/well in 96-well format. Thawing, seeding and culturing can be performed according to supplier's instructions.

Oligonucleotide treatment: Oligonucleotide compositions 14, 39, and TUG1 (negative control). Concentration: 20 μM for oligonucleotide composition 14, 18.9 μM for oligonucleotide composition 39, and 20 μM for negative control oligonucleotide targeting TUG1. Vehicle: 2% (v/v) TE buffer without oligonucleotides. Oligonucleotides were added to cells on day 7 after seeding the cells (by gymnotic uptake) and removed by medium refreshment on day 9 (48 hours total incubation). Cells were collected and lysed on day 7 after seeding the cells (baseline) and 3, 7, 10, 14, and 21 days after removal of oligonucleotide compositions. For mRNA collection, treatments were conducted in triplicate for each condition, with a separate plate for each collection time point. For protein collection, treatments were conducted in three triplicates for each condition with a separate plate for each collection time point.

Negative control: <A*C*C*A*G*>T*G*C*A*T*T*C*A*T*T*<C*G*A*G*T> (SEQ ID NO: 185); all nucleosides are DNA nucleosides except those in < > which are 2′-MOE modified, and each * independently represents a phosphorothioate internucleotidic linkage.

mRNA Harvesting: Cells were harvested using Cells-to-Ct 1-Step TaqMan kit (Thermo Fisher), according to supplier's instructions (25 μL/well lysis buffer) on day 7 after seeding the cells (baseline) and at 3, 7, 10, 14, and 21 days after removal of oligonucleotide compositions.

RT-PCR: 15% of Cells-to-Ct lysate samples and the primer-probe sets in the table below, as single-plex reactions, in 384-well format, in technical duplicates. TaqMan Gene Expression Assay using LightCycler 480 equipment (Roche). (CAPN2 and RPLP0 Taqman assays for control and test oligonucleotide samples.)

| Primer-probe Set | Supplier | Assay ID |
|---|---|---|
| Human CAPN2 FAM | Thermo Fisher | Hs00965097_m1 |
| (target) | | |
| Human RPLP0 VIC_PL | Thermo Fisher | Hs00420895_gH |
| (housekeeping) | | |

mRNA Data Analysis: Cp values were determined using the second derivative method (Lightcycler software). % knockdown of gene expression was determined by Pfaffl method to account for primer efficiency mismatch: RQ=(E_{target})^{ΔC(target)}/(E_{reference})^{ΔCt(reference)} (normalized to negative control: TUG1), wherein ΔCt (target)=Ct (target gene in calibrator)−Ct (target gene in tests) and ΔCt (reference)=Ct (reference gene in calibrator)−Ct (reference gene in tests).

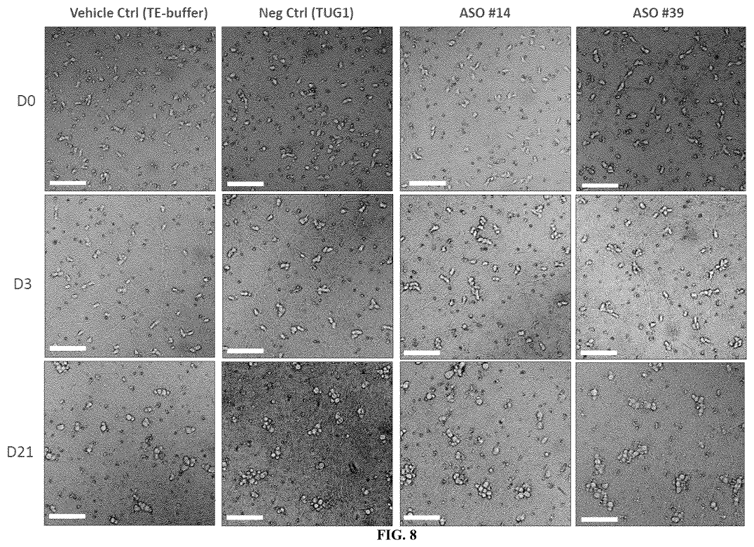

Treatment of cells with vehicle (TE buffer) or negative control oligonucleotide targeting TUG1 resulted in only minor changes in housekeeping (RPLP0) and target (CAPN2) gene Cp values. Small variability in Cp values observed within replicate samples for CAPN2 and RPLP0 upon treatment with vehicle (TE buffer) or negative control oligonucleotide targeting TUG1, likely due to well-to-well variability in cell viability. Cell viability was assessed by Hoechst staining (allowing for counting of cell nuclei) and brightfield imaging. No major cytotoxicity was observed. Some variation in cell viability was observed between individual wells, but average cell nuclei counts were not significantly different upon treatment with oligonucleotides targeting CAPN2 as compared to treatment with vehicle (TE buffer) or negative control oligonucleotide targeting TUG1. Additionally, as displayed in FIG. 8, no significant effects on cell morphology were observed upon treatment with vehicle (TE buffer), negative control oligonucleotide targeting TUG1, or oligonucleotides targeting CAPN2. Further, cell morphology did not significantly differ among treatment conditions to at least day 21 post-removal of oligonucleotide. Formation of cell clumps was observed at day 21 for oligonucleotides targeting CAPN2, and cell clump formation was also observed in control conditions. Neuronal network formation was observed in all treatment conditions.

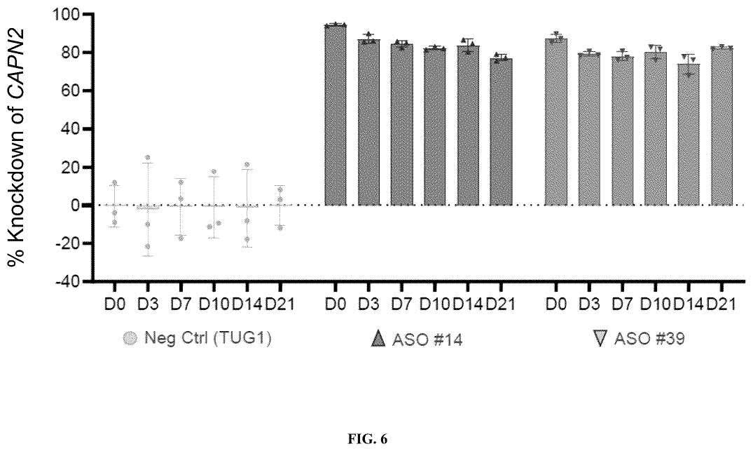

As shown in FIG. 6 and the table below, calpain-2 (CAPN2) mRNA knockdown was confirmed. Oligonucleotide 14 can provide about 94% knockdown of calpain-2 mRNA at 20 μM on day 0 post-removal of oligonucleotide and about 77% knockdown of calpain-2 mRNA at 20 μM on day 21 post-removal of oligonucleotide. Oligonucleotide 39 can provide about 87% knockdown of calpain-2 mRNA at 18.9 μM on day 0 post-removal of oligonucleotide and about 82% knockdown of calpain-2 mRNA at 18.9 μM on day 21 post-removal of oligonucleotide. Both oligonucleotide 14 and oligonucleotide 39 demonstrated persistent knockdown of calpain-2 mRNA from day 0 post-removal of oligonucleotide to at least day 21 post-removal of oligonucleotide. The negative control oligonucleotide targeting TUG1 did not display knockdown of calpain-2 mRNA levels. Among other things, data of the present Example confirm that provided technologies can provide persistent knockdown of calpain-2 mRNA, e.g., following removal or washout of the oligonucleotide or composition.

Protein Harvesting: Cell lysates prepared using RIPA lysis buffer (30 μL/well), including Halt protease inhibitor cocktail (Thermo Fisher). Three wells per replicate were pooled to ensure obtainment of sufficient amounts for protein detection. Three replicates were harvested per condition per time point. Protein yield was assessed by BCA assay.

Protein Detection: Calpain-2 protein was detected using western blot via Jess equipment (ProteinSimple) according to supplier's instructions. Two replicates for each condition and time point were examined. Calpain-2 antibody (EPR5977/ab126600; Abeam) was used at a 1:6 dilution.

Protein Data Analysis: Chemiluminescence-based detection of target immunoreactivity. Signal analysis for area under curve (AUC), detected molecular (MW), and signal-to-noise ratios using Compass software. Quantification of % target protein knockdown, normalized for total protein.

Calpain-2 protein levels were not affected by vehicle (TE buffer) or negative control treatment as compared to untreated cells at 48 hours after treatment. Some variation in calpain-2 protein levels between replicates was observed with some conditions at some time points, e.g., in some cases likely due to well-to-well variability in cell viability. For the negative control for oligonucleotide 39, slight calpain-2 protein signal burn-out was observed in samples for days 10, 14, and 21 as a result of high protein loading and insufficient substrate, which may result in an under-estimation of protein knockdown. Cell viability was assessed by Hoechst staining (allowing for counting of cell nuclei) and brightfield imaging. No major cytotoxicity was observed. Some variation in cell viability was observed between individual wells, but average cell nuclei counts were not significantly different upon treatment with oligonucleotides targeting CAPN2 as compared to treatment with vehicle (TE buffer) or negative control oligonucleotide targeting TUG1. Additionally, as displayed in FIG. 8, no significant effects on cell morphology were observed upon treatment with vehicle (TE buffer), negative control oligonucleotide targeting TUG1, or oligonucleotides targeting CAPN2. Further, cell morphology did not significantly differ among treatment conditions to at least day 21 post-removal of oligonucleotide. Formation of cell clumps was observed at day 21 for oligonucleotides targeting CAPN2, and cell clump formation was also observed in control conditions. Neuronal network formation was observed in all treatment conditions.

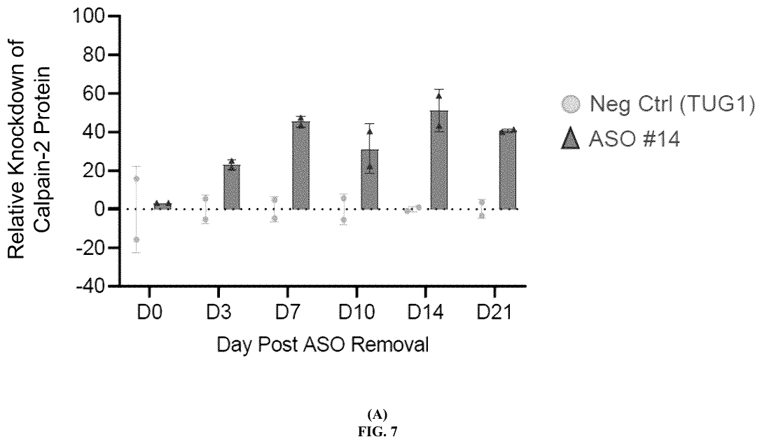

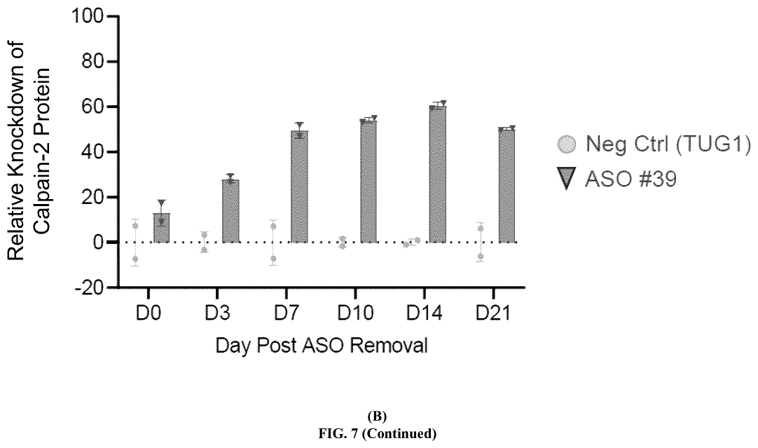

As shown in FIG. 7 and the table below, calpain-2 protein knockdown was confirmed. Oligonucleotide 14 can provide about 51% knockdown of calpain-2 protein at 20 μM at day 14 post-removal of oligonucleotide, as shown in FIG. 7A. Oligonucleotide 39 can provide about 60% knockdown of calpain-2 protein at 18.9 μM at day 14 post-removal of oligonucleotide, as shown in FIG. 7B. Both oligonucleotide 14 and oligonucleotide 39 demonstrated persistent calpain-2 protein knockdown from day 7 post-removal of oligonucleotide and maintained persistent calpain-2 protein knockdown to at least day 21 post-removal of oligonucleotide. The negative control oligonucleotide targeting TUG1 did not display knockdown of calpain-2 protein levels. Among other things, data of the present Example confirm that provided technologies can provide persistent knockdown of calpain-2 protein, e.g., following removal or washout of the oligonucleotide or composition.

| Oligonucleotide 14 | | | Oligonucleotide 39 | | |
|---|---|---|---|---|---|
| | mRNA | Protein | | mRNA | Protein |
| Day | % KD | % KD | Day | % KD | % KD |
| 0 | 94.47 | 3.25 | 0 | 87.40 | 13.10 |
| 3 | 87.12 | 23.32 | 3 | 79.20 | 28.09 |
| 7 | 84.36 | 45.47 | 7 | 78.15 | 49.51 |
| 10 | 82.16 | 31.39 | 10 | 80.24 | 54.18 |
| 14 | 83.80 | 51.07 | 14 | 73.98 | 60.46 |
| 21 | 77.25 | 40.75 | 21 | 82.35 | 50.17 |

Various other technologies are available for assessing properties and/or activities of provided technologies in accordance with the present disclosure. For example, reduction of levels of calpain-2 polypeptides can be assessed through western blot or immunostaining. In some embodiments, provided oligonucleotides and compositions are assessed for reduction of calpain-2 activation. In some embodiments, provided oligonucleotides and compositions are assessed for reduction of calpain-2 protease activity. In some embodiments, provided oligonucleotides and compositions are assessed for reduction of neuronal degeneration, e.g., through using one or more cell models. In some embodiments, provided oligonucleotides and compositions are assessed for enhanced recovery from axotomy.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. An oligonucleotide, wherein:
the base sequence of the oligonucleotide comprises 10 or more contiguous nucleobases of ATCAGTTTCTGTAGGCTTCC (SEQ ID NO: 1), GGCATACTGGTTCAGTTGAT (SEQ ID NO 2), GCTCAGGTCAGGCAGTGGTT (SEQ ID NO: 3), GAGAGCCTTTTTGCAGAGCT (SEQ ID NO: 4), TCCAGCTCTGTGCCTCTAGT (SEQ ID NO: 5), GTTCCAGCTTGGGCAGTTGT (SEQ ID NO: 6), or GGAAGCTTAGTCCTTGGCTG (SEQ ID NO: 7), wherein each T is optionally and independently replaced with U; and
the oligonucleotide comprises a modified nucleobase, a modified sugar or a modified internucleotidic linkage.

2. The oligonucleotide of claim 1, wherein the base sequence of the oligonucleotide is ATCAGTTTCTGTAGGCTTCC (SEQ ID NO: 8), GGCATACTGGTTCAGTTGAT (SEQ ID NO: 9), GCTCAGGTCAGGCAGTGGTT (SEQ ID NO: 10), GAGAGCCTTTTTGCAGAGCT (SEQ ID NO: 11), TCCAGCTCTGTGCCTCTAGT (SEQ ID NO: 12) GTTCCAGCTTGGGCAGTTGT (SEQ ID NO: 13), or GGAAGCTTAGTCCTTGGCTG (SEQ ID NO: 14).

3. The oligonucleotide of claim 2, wherein the oligonucleotide comprises a 5′-wing-gap-wing-3′ structure.

4. The oligonucleotide of claim 2, wherein there are about 3-10 nucleosides in the 5′-wing, optionally wherein there are 5 nucleosides in the 5′-wing.

5. The oligonucleotide of claim 2, wherein each sugar in the 5′-wing is independently a modified sugar.

6. The oligonucleotide of claim 2, wherein a sugar in a 5′-wing is a 2′-OR^{s} modified sugar wherein R^{s} is C₁₋₆ aliphatic;
wherein a sugar in a 5′-wing is a 2′-MOE modified sugar;
wherein a sugar in a 5′-wing is a 2′-OMe modified sugar, and/or
wherein a sugar in a 5′-wing is a bicyclic sugar, optionally wherein the bicyclic sugar is a LNA sugar or cEt sugar.

7. The oligonucleotide of claim 2, wherein each sugar in a 5′-wing is independently a 2′-OR^{s} modified sugar wherein R^{s} is C₁₋₆ aliphatic or
wherein each sugar in a 5′-wing is independently a 2′-MOE modified sugar.

8. The oligonucleotide of claim 2, wherein there are 10 nucleosides in the gap.

9. The oligonucleotide of claim 2, wherein each sugar in the gap is independently a natural DNA sugar.

10. The oligonucleotide of claim 2, wherein the gap contains no cytosine and/or wherein the gap comprises one or more 5-methylcytosine.

11. The oligonucleotide of claim 2, wherein there are about 3-10 nucleosides in the 3′-wing, optionally wherein there are 5 nucleosides in the 3′-wing.

12. The oligonucleotide of claim 2, wherein each sugar in the 3′-wing is independently a modified sugar.

13. The oligonucleotide of claim 2, wherein a sugar in a 3′-wing is a 2′-OR^{s} modified sugar wherein R^{s} is C₁₋₆ aliphatic;
wherein a sugar in a 3′-wing is a 2′-MOE modified sugar;
wherein a sugar in a 3′-wing is a 2′-OMe modified sugar; and/or
wherein a sugar in a 3′-wing is a bicyclic sugar, optionally wherein the bicyclic sugar is a LNA sugar or cEt sugar.

14. The oligonucleotide of claim 2, wherein each sugar in a 3′-wing is independently a 2′-OR^{s} modified sugar wherein R^{s} is C₁₋₆ aliphatic or
wherein each sugar in a 3-wing is independently a 2′-MOE modified sugar.

15. The oligonucleotide of claim 2, wherein the oligonucleotide comprises a modified internucleotidic linkage, optionally wherein the modified internucleotidic linkage is a phosphorothioate internucleotidic linkage.

16. The oligonucleotide of claim 2, wherein each internucleotidic linkage is independently a modified internucleotidic linkage and/or
wherein each internucleotidic linkage is independently a phosphorothioate internucleotidic linkage.

17. An oligonucleotide having the structure of:
(SEQ ID NO: 116)
/52MOErA/*/i2MOErT/*/i2MOErC/*/i2MOErA/*/i2MOErG/
*T*T*T*/iMe-dC/*T*G*T*A*G*G*/i2MOErC/*/i2MOErT/*/
i2MOErT/*/i2MOErC/*/32MOErC/,
(SEQ ID NO: 141)
/52MOErG/*/i2MOErG/*/i2MOErC/*/i2MOErA/*/i2MOErT/
*A*/iMe-dC/*T*G*G*T*T*/iMe-dC/*A*G*/i2MOErT/*/
i2MOErT/*/i2MOErG/*/i2MOErA/*/32MOErT/,
(SEQ ID NO: 173)
/52MOErG/*/i2MOErC/*/i2MOErT/*/i2MOErC/*/i2MOErA/
*G*G*T*/iMe-dC/*A*G*G*/iMe-dC/*A*G*/i2MOErT/*/
i2MOErG/*/i2MOErG/*/i2MOErT/*/32MOErT/,
(SEQ ID NO: 114)
/52MOErG/*/i2MOErA/*/i2MOErG/*/i2MOErA/*/i2MOErG/
*/iMe-dC/*/iMe-dC/*T*T*T*T*T*G*/iMe-dC/*A*/
i2MOErG/*/i2MOErA/*/i2MOErG/*/i2MOErC/*/32MOErT/,
(SEQ ID NO: 179)
/52MOErT/*/i2MOErC/*/i2MOErC/*/i2MOErA/*/i2MOErG/
*/iMe-dC/*T*/iMe-dC/*T*G*T*G*/iMe-dC/*/iMe-dC/
*T*/i2MOErC/*/i2MOErT/*/i2MOErA/*/i2MOErG/*/
32MOErT/,
(SEQ ID NO: 117)
/52MOErG/*/i2MOErT/*/i2MOErT/*/i2MOErC/*/i2MOErC/
*A*G*/iMe-dC/*T*T*G*G*G*/iMe-dC/*A*/i2MOErG/*/
i2MOErT/*/i2MOErT/*/i2MOErG/*/32MOErT/,
or
(SEQ ID NO: 138)
/52MOErG/*/i2MOErG/*/i2MOErA/*/i2MOErA/*/i2MOErG/
*/iMe-dC/*T*T*A*G*T*/iMe-dC/*/iMe-dC/*T*T*/
i2MOErG/*/i2MOErG/*/i2MOErC/*/i2MOErT/*/32MOErG/,
or a salt thereof, wherein: 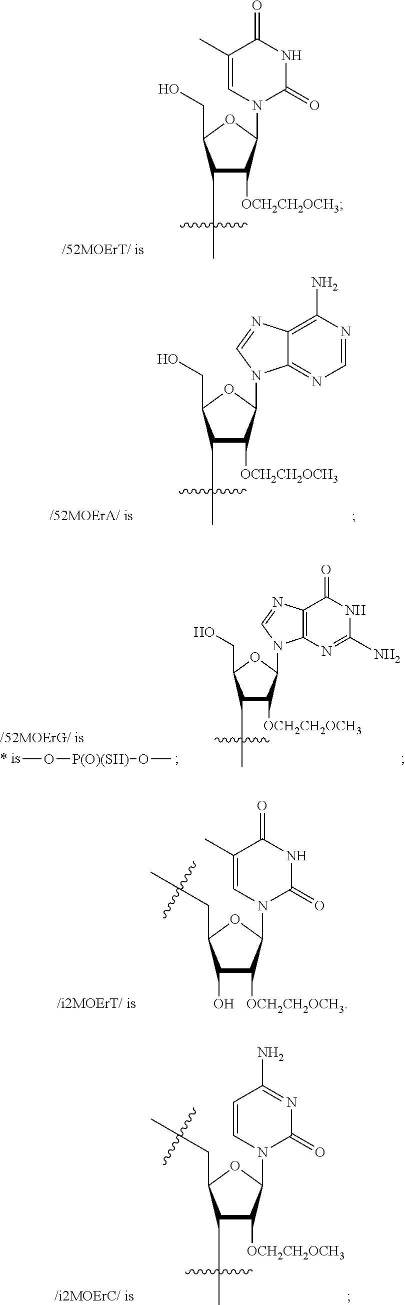 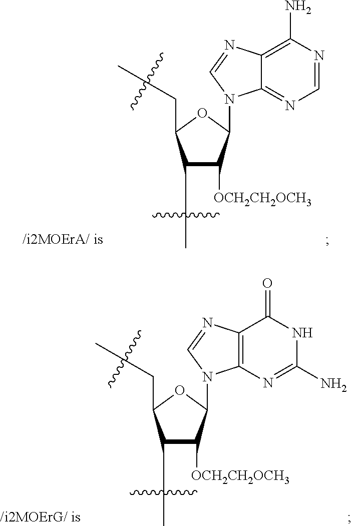
each of A, T and G is independently deoxyadenosine, thymidine, and deoxyguanosine, respectively:

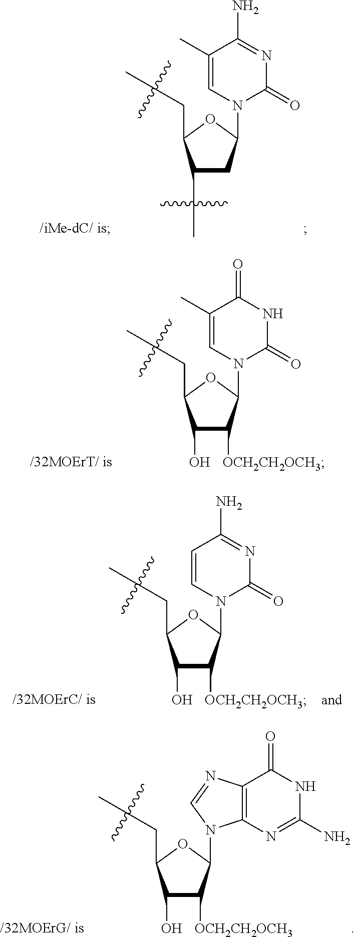

18. The oligonucleotide of claim 17, wherein the oligonucleotide is a pharmaceutically acceptable salt, optionally wherein the oligonucleotide is a sodium salt.

19. A composition comprising an oligonucleotide of claim 17 and one or more diastereomers of the oligonucleotide with respect to chiral linkage phosphorus.

20. A composition comprising:
an oligonucleotide or a salt thereof, and
one or more diastereomers of the oligonucleotide with respect to chiral linkage phosphorus, or one or more salts of the diastereomers,
wherein the oligonucleotide is
(SEQ ID NO: 116)
/52MOErA/*/i2MOErT/*/i2MOErC/*/i2MOErA/*/i2MOErG/
*T*T*T*/iMe-dC/*T*G*T*A*G*G*/i2MOErC/*/i2MOErT/*/
i2MOErT/*/i2MOErC/*/32MOErC/,
(SEQ ID NO: 141)
/52MOErG/*/i2MOErG/*/i2MOErC/*/i2MOErA/*/i2MOErT/
*A*/iMe-dC/*T*G*G*T*T*/iMe-dC/*A*G*/i2MOErT/*/
i2MOErT/*/i2MOErG/*/i2MOErA/*/32MOErT/,
(SEQ ID NO: 173)
/52MOErG/*/i2MOErC/*/i2MOErT/*/i2MOErC/*/i2MOErA/
*G*G*T*/iMe-dC/*A*G*G*/iMe-dC/*A*G*/i2MOErT/*/
i2MOErG/*/i2MOErG/*/i2MOErT/*/32MOErT/,
(SEQ ID NO: 179)
/52MOErT/*/i2MOErC/*/i2MOErC/*/i2MOErA/*/i2MOErG/
*/iMe-dC/*T*/iMe-dC/*T*G*T*G*/iMe-dC/*/iMe-dC/
*T*/i2MOErC/*/i2MOErT/*/i2MOErA/*/i2MOErG/*/
32MOErT/,
(SEQ ID NO: 117)
/52MOErG/*/i2MOErT/*/i2MOErT/*/i2MOErC/*/i2MOErC/
*A*G*/iMe-dC/*T*T*G*G*G*/iMe-dC/*A*/i2MOErG/*/
i2MOErT/*/i2MOErT/*/i2MOErG/*/32MOErT/,
(SEQ ID NO: 138)
/52MOErG/*/i2MOErG/*/i2MOErA/*/i2MOErA/*/i2MOErG/
*/iMe-dC/*T*T*A*G*T*/iMe-dC/*/iMe-dC/*T*T*/
i2MOErG/*/i2MOErG/*/i2MOErC/*/i2MOErT/*/32MOErG/,
or
(SEQ ID NO: 114)
/52MOErG/*/i2MOErA/*/i2MOErG/*/i2MOErA/*/i2MOErG/
*/iMe-dC/*/iMe-dC/*T*T*T*T*T*G*/iMe-dC/*A*/
i2MOErG/*/i2MOErA/*/i2MOErG/*/i2MOErC/*/32MOErT/,
wherein: 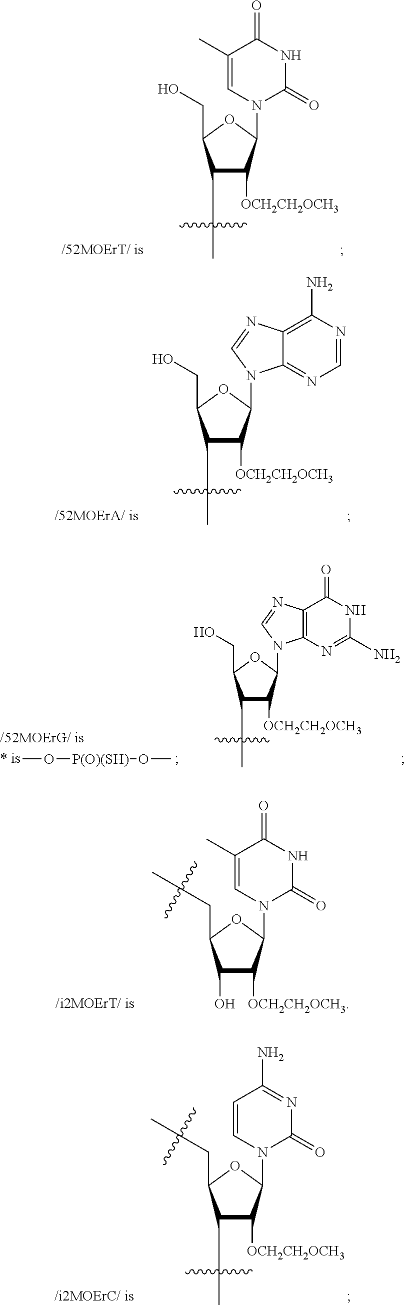 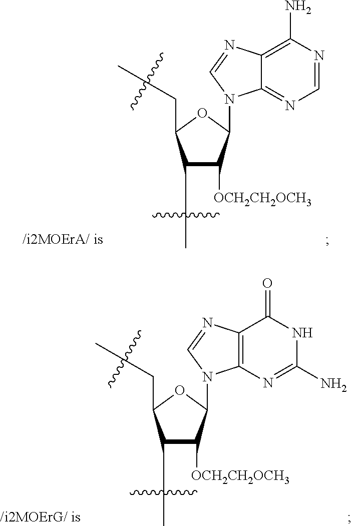
each of A, T and G is independently deoxyadenosine, thymidine, and deoxyguanosine, respectively:

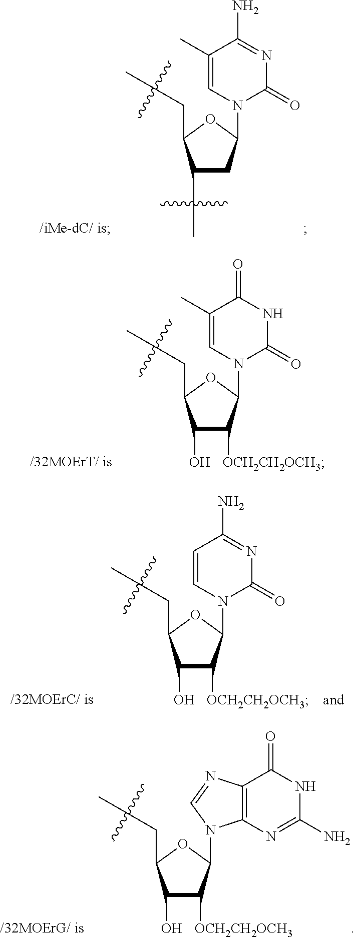

21. The composition of claim 20, wherein for each chiral linkage phosphorus, the percentage of the Rp configuration is independently about 20%-80%, about 30%-70%, about 40%-60%, about 45%-55%, or about 50%.

22.-24. (canceled)

25. A pharmaceutical composition comprising the oligonucleotide of claim 17 and a pharmaceutically acceptable carrier.

26. The composition of claim 25, wherein the composition comprises one or more pharmaceutically acceptable salts of an oligonucleotide.

27. (canceled)

28. The composition of claim 25, wherein the pharmaceutically acceptable carrier is a buffer, buffered saline, or artificial cerebrospinal fluid.

29. A method for reducing one or more of the following: level of calpain-2 mRNA, level of calpain-2 polypeptide, and/or level of calpain-2 activity in a system, comprising administering or delivering to the system an effective amount of the composition of claim 25.

30.-31. (canceled)

32. The method of claim 29, wherein the system expresses calpain-2 mRNA.

33. The method of claim 29, wherein the system is or comprises a cell, a tissue, an organ, brain or a portion thereof, an organism, a subject, or a human.

34.-44. (canceled)

45. A method for preventing or treating a condition, disorder or disease, comprising administering or delivering to a subject in need thereof an effective amount of the composition of claim 25.

46.-49. (canceled)

50. The method of claim 45, wherein the condition, disorder or disease is a neurodegenerative condition, disorder, or disease, amyotrophic lateral sclerosis, peripheral neuropathy, peripheral neuropathy induced by chemotherapy, Parkinson's disease, Huntington's disease, Alzheimer's disease, frontotemporal dementia, traumatic brain injury, progressive supranuclear palsy, corticobasal degeneration, Wolfram syndrome, Friedreich's ataxia, multiple system atrophy, spinal cerebellar ataxia, spinal muscular atrophy, Pick's disease, progressive motor atrophy, stroke, concussion, intracerebral hemorrhage, acute glaucoma, seizure, spinal cord injury, and/or a condition, disorder, or disease associated with Wallerian degeneration.

51. The method of claim 45, wherein NF-L excretion is reduced.

52. (canceled)

53. The method of claim 45, wherein neuritic degeneration is reduced.

54.-55. (canceled)

56. The method of claim 45, wherein the oligonucleotide or composition is administered or delivered intrathecally and/or intravenously.

57. (canceled)
